(19) United States
(12) Patent Application Publication
(10) Pub. No.: US 2025/0011780 A1
(43) Pub. Date: Jan. 9, 2025

(54) COMPOSITIONS OF HUMAN HEART DERIVED EXTRACELLULAR VESICLES AND USES THEREOF

(71) Applicant: UNIVERSITY OF OTTAWA HEART INSTITUTE, Ottawa (CA)

(72) Inventors: Darryl Raymond DAVIS, Ottawa (CA); Sandrine PARENT, Ottawa (CA)

(21) Appl. No.: 18/709,356

(22) PCT Filed: Nov. 11, 2022
(86) PCT No.: PCT/CA2022/051669

§ 371 Date: May 10, 2024

Related U.S. Application Data
(60) Provisional application No. 63/278,518, filed on Nov. 12, 2021.

(51) Int. Cl. C12N 15/113; A61K 9/51; A61P 9/06; A61P 29/00; B82Y 5/00; C12N 5/077

(52) U.S. Cl. CPC C12N 15/113; A61K 9/5176; A61P 9/06; A61P 29/00; C12N 5/0657; B82Y 5/00; C12N 2310/141; C12N 2320/32

(57) ABSTRACT

Compositions of human heart derived extracellular vesicles are provided. These compositions may comprise extracellular vesicles and a biologically or pharmaceutically acceptable adjuvant or carrier, said extracellular vesicles may comprise one or more of the following characteristics: extracellular vesicles isolated from human heart cells, a polydisperse population of particles, one or more cytosolic markers, one or more transmembrane markers, acetylcholinesterase activity, substantially lacking or devoid of one or more contaminants, unique miRNA transcripts, and a unit dosage of extracellular vesicles. Uses of compositions of human heart derived extracellular vesicles thereof in treating and preventing atrial arrhythmias, atrial inflammation, atrial fibrosis and atrial fibroblast proliferation, are also provided.

### FIELD OF INVENTION

The present invention relates generally to treatment or prevention of atrial arrhythmias. More specifically, the present invention relates to compositions comprising extracellular vesicles, and uses and methods for treatment of atrial arrhythmias.

### BACKGROUND

Atrial arrhythmias, including atrial fibrillation, are the most common heart rhythm disturbance in the world-afflicting almost 38 million people worldwide (Benjamin, Levy et al. 1994; Furberg, Psaty et al. 1994; Go, Hylek et al. 2001). Subjects with atrial arrhythmia may present with symptoms that can significantly affect quality of life, including but not limited to, general fatigue, rapid heartbeat, irregular heartbeat, dizziness, shortness of breath, anxiety, weakness, confusion, faintness, sweating, chest pain, chest pressure, syncope, palpitations, and chest fluttering. Atrial arrhythmias occur when the upper chamber of the heart (atrium) beats irregularly and out of sync with the lower chamber of the heart (ventricle), resulting in an irregular heartbeat. Ventricular arrhythmias can also occur when the ventricle beats out of sync with the atrium, however, such arrhythmias differ significantly from atrial arrhythmias in their root cause, treatment, symptoms and mortality. As such, many treatments to suppress ventricular arrhythmias are ineffective in atrial arrhythmias (Ledan 2020). Specifically, drugs that slow electrical conductance are contra-indicated in ventricle arrhythmia patients due to increased risk of death (Friberg 2018).

Though atrial arrhythmias are not inherently life threatening, they increase the risk of blood clots and the risk of stroke by 5-fold (McRae, Kapoor et al. 2019). Without significant advances in either the treatment or prevention of atrial arrhythmias, this will also correspond with increases in hospitalization and stroke (Go, Hylek et al. 2001; Seo, Michie et al. 2020). Rhythm and rate control medications are common drug therapy treatments for atrial arrhythmias, however, they often fail and their use is limited by off target effects on blood pressure and heart function.

Given the limitation in treating and preventing atrial arrhythmias with current therapies, recent preclinical work has focused on biological therapies to target mechanisms thought to trigger atrial arrhythmias including reducing inflammation and modifying atrial electrophysiology (McRae, Kapoor et al. 2019; Seo, Michie et al. 2020).

Alternative, additional and/or improved treatments for atrial arrhythmias is desirable.

### SUMMARY OF INVENTION

Traditionally, atrial arrhythmias are treated with drugs that control how the heart beats, restoring it to a normal rhythm. These drugs have modest efficacy and significant side effects, as well as waning suppression over time. Other treatment options include electrical cardioversion, catheter procedures and surgical procedures. Catheter ablation is commonly used, however, success rates vary considerably and serious complications are possible (Lycke, O'Neill et al. 2021). A limitation of current drug approaches is they may only target heart rate, however, this does not address others mechanisms that can potentially lead to atrial arrhythmias such as fibrosis, fibroblast proliferation and inflammation (Jost, Christ et al. 2021). Biological based treatment options are emerging to treat atrial arrhythmia, but to date have not translated into clinical trials.

Extracellular vesicles are naturally secreted, replication incompetent particles, bound by a lipid bilayer carrying heterogeneous constituents, including but not limited to organelles, nucleic acids, proteins, lipids, and metabolites, which may have the potential to target multiple mechanisms relevant to suppressing atrial arrhythmia. Extracellular vesicle composition varies across tissues, cells, and physiological parameters; therefore obtaining a composition effective in treatment of specific disorders has proved elusive, however, clinical trials have been initiated for several extracellular vesicle related therapeutics (Ciferri, Quarto et al. 2021). The inventors hypothesized that extracellular vesicles secreted by human heart cells may treat or prevent atrial fibrosis, and atrial inflammation, which in turn are thought to contribute to development of atrial arrhythmia. Accordingly, studies as described herein below were performed to investigate whether extracellular vesicles derived from human heart cells may provide treatment for atrial arrhythmias such as, for example, atrial fibrillation.

The teachings herein, provide compositions and methods comprising extracellular vesicles derived from human heart cells, as well as uses thereof in treating and preventing atrial arrhythmias, atrial inflammation, atrial fibrosis and atrial fibroblast proliferation.

In an embodiment, there is provided herein a composition comprising extracellular vesicles and a biologically or pharmaceutically acceptable adjuvant or carrier, said extracellular vesicles may comprise one or more of the following characteristics:
extracellular vesicles isolated from human heart cells;
a polydisperse population of particles ranging from about 1 nm to about 1000 nm, or any values defining a range therein;
one or more cytosolic markers defined by ALIX, any ANXA polypeptide, any ACT polypeptide, TSG101, any CHMP polypeptide, PDCD6IP, VPS4A, VPS4B, ARRDC1, any CAV polypeptide, any EHD polypeptide, RHOA, HSPA8, HSPA1A, any actin polypeptide, any tubulin polypeptide, GAPDH, HSP90AB1, ARF6, SDCBP, MAPT, HSC70, HSP70, HSP84, HSPA8 and TSG101, or a combination thereof;
one or more transmembrane markers defined by CD3, CD9, CD14, CD37, CD41, CD45, CD47, CD53, CD55, CD59, CD61, CD63, CD73, CD81, CD82, CD90, any GNA polypeptide, HLA-A, HLA-B, HLA-C, HLA-DR, HLA-DP, HLA-DQ, any H2-A polypeptide H2-K, H2-D, H2-Q, any ITGA polypeptide, any ITGB polypeptide, TFR2, LAMP1, LAMP2, any SDC polypeptide, BSG, ADAM10, SHH, TSPAN8, PECAMI, ERBB2, EPCAM, CDC42a, GYPA, AChE-S, AChE-E, AβA4, APP, ABCC1, FLOT1, FLOT2, ICAM1, EpCam, or a combination thereof,
acetylcholinesterase activity;
substantially lacking or devoid of one or more of the group comprising GM130, APOA1, APOA2, APOB, APOB100, ALB, ribosomal proteins, UMOD and protein/nucleic acid aggregates;
1-1000 unique miRNA transcripts; and
a unit dosage of extracellular vesicles comprising from about 10² to about 10²⁰ particles per millilitre, or any values defining a range therein.

In another embodiment of the composition above, the human heart cells may be autologous or allogenic to the subject receiving the composition.

In another embodiment of the compositions above, said extracellular vesicles may be absorbed by immune cells, said immune cells including, but not limited to, lymphocytes, T-cells, B-cells, NK cells, monocytes, macrophages, and neutrophils. In certain embodiments, said extracellular vesicles may decrease inflammasome activation. In certain embodiments, said extracellular vesicles may decrease immune cell inflammasome activation.

In another embodiment of any of the compositions above, the extracellular vesicles may be absorbed by human heart cells. In another embodiment, the human heart cells absorbing the extracellular vesicles may be cardiomyocytes, endothelial cells, immune cells and/or fibroblasts. In certain embodiments, absorption by human heart cells may be determined by transfer of protein from the extracellular vesicle to the human heart cells. It is contemplated that in certain embodiments, transfer of protein from the extracellular vesicle to the human heart cells may be the transfer of one or more proteins comprising: an exogenous protein, a fluorescent protein, a luminescent protein, a protein capable of producing a colorimetric response, a protein tagged with a group capable of fluorescence, a protein tagged with a group capable of luminescence, a protein tagged with a group capable of colorimetric response, a protein not detectable or absent from human heart cells or any combination thereof. In certain further embodiments, the protein tagged with a group capable of fluorescence may be CD63. In certain embodiments, the transfer of membrane lipids from the extracellular vesicle to the human heart cells may determine absorption by the human heart cells. In further embodiments, transfer of membrane lipids from the extracellular vesicle to the human heart cells may be measured using one or more techniques comprising: 1,1′-Dioctadecyl-3,3,3′,3′-Tetramethylindocarbocyanine Perchlorate (Dil), PKH67 PKH26, any fluorescent dye capable of membrane localization, any luminescent dye capable of membrane localization, any colourmetric dye capable of membrane localization, any functional equivalents and any combination thereof. In a still further embodiment, transfer of membrane lipids from the extracellular vesicle to the human heart cells may be measured using DiI.

In another embodiment of any compositions above, the human heart cells may be derived from a heart biopsy. In certain embodiments, the biopsy may contain one or more of the following heart layers: endocardial, myocardial, epicardial or any combination thereof. In another embodiment, the human heart cells may be derived from myocardial tissue. In certain embodiments, the human heart cells may be derived from an atrial appendage. In certain embodiments, the human heart cells may be derived from a left atrial appendage. In certain embodiments, the human heart cells may be derived from ventricular tissue. In certain embodiments, the human heart cells may be derived from an entire heart.

In another embodiment of any compositions above, extracellular vesicles may be isolated from one or more of the following: atrial explant derived stem cells, atrial explant derived cells, heart explant derived stem cells; human heart explant derived cells, heart explant derived cells; cardiac explant derived stem cells; cardiac explant derived cells; heart cells differentiated from induced pluripotent stem cells; heart cells differentiated from embryonic stem cells; or heart cells derived from any cell type capable of differentiation into heart cells.

In another embodiment of any compositions above, the human heart cells may be cryopreserved.

In another embodiment of any compositions above, said extracellular vesicles may be isolated from an immortalized cell line. In further embodiments of any compositions above, said extracellular vesicles may be isolated from immortalized heart explant derived cells. In certain embodiments, heart explant derived cells may be immortalized with one or more of the following: viral gene, viral gene product, genetic mutation, telomerase reverse transcriptase expression, SV40, or equivalent techniques.

In another embodiment of any compositions above, the human heart cells may have been grown in vitro. In a further embodiment, the human heart cells may have been expanded in vitro. In a still further embodiment, the human heart cells may have been grown in vitro using Good Manufacturing Practices (GMP) conditions comprising:
controlled physiological cell culture conditions, said conditions comprising continuous atmosphere around 5% oxygen, and around 5% carbon dioxide, relative humidity around 80% RH, and temperature around 37° C.;
serum-free, xenogen-free growth media; and
extracellular vesicle depleted fetal bovine serum.

In certain embodiments, the human heart cells may have been expanded in vitro using GMP conditions. In another embodiment, the human heart cells may have been expanded in vitro using a GMP compliant enzyme to disassociate cells from culture plates, wherein said enzyme may be one or more of TrypLE™ Select, collagenase I and collagenase II.

In another embodiment of any compositions above, the extracellular vesicles may be isolated after about 1 hour to about 196 hours incubation. In certain embodiments, the extracellular vesicles may be isolated after about 48 hours incubation, or more.

In another embodiment of any compositions above, the composition may be substantially immunologically inert.

In another embodiment of any compositions above, the extracellular vesicles may be a polydisperse population of particles ranging from about 75 nm to about 500 nm, any values defining a range therein, for example, but not limited to about 95 nm to about 250 nm. In certain embodiments, the extracellular vesicles may be a polydisperse population that comprises an average diameter of about 75 nm to about 200 nm. In further embodiments, the extracellular vesicles may be a polydisperse population that comprises an average diameter of about 132 nm.

In another embodiment of any compositions above, the one or more cytosolic markers may be ALIX, ANXA5 and TSG101, or any combination thereof.

In another embodiment of any compositions above, the one or more transmembrane markers may be CD9, CD61, CD63, CD81, FLOT1, ICAM1, EpCam, or any combination thereof. In further embodiments, CD81 may be expressed on an equal or fewer number of particles than particles expressing CD9 or CD63. In certain further embodiments, CD81 may be expressed on about half or fewer the number of particles compared to the number of particles expressing CD63.

In another embodiment of any compositions above, acetylcholinesterase activity may be measured using FluoroCet Exosome Quantitation kit or equivalent methodology. In certain embodiments, acetylcholinesterase activity may be used to quantify extracellular vesicle particle number.

In another embodiment of any compositions above, the extracellular vesicles may be substantially lacking or devoid of GM130.

In another embodiment of any compositions above, the miRNA may be 10-250 unique transcripts. In certain embodiments, the miRNA may be 60-85 unique transcripts. In further embodiments, the miRNA may be one or more of miR-23a-3p, miR-199a-3p+miR-199b-3p, miR-4454+miR-7975, let-7a-5p, let-7b-5p, miR-125b-5p, miR-100-5p, miR-29b-3p, miR-21-5p, miR-191-5p, miR-199b-5p, miR-29a-3p, miR-22-3p, let-7i-5p, miR-181a-5p, miR-25-3p, miR-127-3p, let-7g-5p, miR-15b-5p, miR-320e, miR-221-3p, let-7d-5p, miR-16-5p, miR-424-5p, miR-3180, miR-374a-5p, miR-15a-5p, miR-130a-3p, miR-376a-3p, miR-199a-5p, miR-222-3p, miR-4286, miR-4516, miR-34a-5p, miR-1255a, miR-365a-3p+miR-365b-3p, miR-323a-3p, let-7c-5p, miR-27b-3p, miR-134-3p, miR-451a, miR-23b-3p, miR-423-5p, miR-28-5p, miR-125a-5p, miR-24-3p, miR-382-5p, miR-1228-3p, miR-20a-5p+miR-20b-5p, let-7e-5p, miR-18a-5p, miR-337-5p, miR-320e, miR-106a-5p+miR-l7-5p, miR-19b-3p, miR-140-5p, let-7f-5p, miR-323a-5p, miR-148a-3p, miR-132-3p, miR-136-5p, miR-376c-3p, miR-379-5p, miR-26a-5p, miR-202-3p, miR-1290, miR-154-5p, miR-214-3p, miR-377-3p, miR-381-3p, miR-188-5p, miR-26b-5p, miR-363-3p, miR-337-3p, miR-137, miR-1973, miR-193a-5p+miR-193b-5p, miR-411-5p, a functional equivalent or a combination thereof. In certain embodiments, the miRNA transcripts may be substantially lacking or devoid of one or more of miR-1, miR-133, miR-328, miR-590 or any combination thereof. In certain embodiments, the miRNA transcripts may be substantially lacking or devoid of one or more of miR-210, miR-146a or a combination thereof.

In another embodiment of any compositions above, the unit dosage of extracellular vesicles may be 10⁸-10¹⁵ particles per millilitre. In a further embodiment, the unit dosage of extracellular vesicles may be 10¹⁰ particles per millilitre.

In another embodiment of any compositions above, the unit dosage of extracellular vesicles may be 10²-10²⁰ particles.

In another embodiment, any compositions above may be for use in the treatment of atrial arrhythmia. In an embodiment the atrial arrhythmia may be selected from a group consisting of atrial fibrillation, post-operative atrial fibrillation, post-infarction atrial fibrillation, thyrotoxicosis, post-viral atrial fibrillation, alcohol-associated atrial fibrillation, drug-induced atrial fibrillation, viral atrial fibrillation, post-viral atrial fibrillation, COVID-19 atrial fibrillation, post-COVID-19 atrial fibrillation, paroxysmal atrial fibrillation, permanent atrial fibrillation, persistent atrial fibrillation, long-term persistent atrial fibrillation, atrial tachycardia, atrial flutter, familial atrial fibrillation, idiopathic atrial fibrillation, lone atrial fibrillation, and any orphan atrial arrhythmia.

In certain embodiments of any compositions above, said extracellular vesicles may be delivered systemically. In other embodiments, said extracellular vesicles may be delivered locally. In certain embodiments, said extracellular vesicles may be delivered intra-myocardially. In certain embodiments, said extracellular vesicles may be delivered intra-atrially. In certain embodiments, said extracellular vesicles may be delivered by catheter. In another embodiment, said extracellular vesicles may be delivered by central venous access device. In another embodiment, said extracellular vesicles may be delivered by peripherally inserted central catheters.

In another embodiment of any compositions above, said extracellular vesicles may reduce or prevent atrial inflammation. In certain embodiments, reduction of atrial inflammation may be quantified histologically, comprising a reduced number of inflammatory cells in the atria of the heart when compared to an untreated subject. In further embodiments, the number of inflammatory cells in the atria may be quantified using one or more markers comprising: hematoxylin and eosin staining, activated T lymphocytes, macrophage infiltration, macrophage polarization, mast cells, neutrophils or any combination thereof. In certain embodiments, said histological reduction in the number of inflammatory cells in the atria may comprise a decrease in the inflammatory cells in the atria ranging from about 1% to about 100% when compared to an untreated subject. In another embodiment, said reduction in atrial inflammation may be a reduced level of cytokines in the heart when compared to an untreated subject.

In certain embodiments, one or more of said cytokines comprising: IL-1, IL-2, IL-3, IL-4, IL-5, IL-6, IL-7, IL-8, IL-9, IL-10, IL-12, IL-11, IL-13, IL-16, IL-17, IL-18, G-CSF, GM-CSF, MCP-1, PDGF-AB, TNF-α, any type I IFN, any type II IFN, any type III IFN or any combination thereof. In another embodiment, said reduction in cytokine level may be quantified using multiplex Luminex® assays, comprising a decrease in one or more of the cytokines by 1% to about 100% amount when compared to an untreated subject. In another embodiment, said reduction in cytokine level may be quantified using enzyme-linked immunosorbent assay (ELISA), comprising a decrease in one or more of the cytokines by 1% to about 100% amount when compared to an untreated subject.

In another embodiment of any of the above compositions, said extracellular vesicles may reduce or prevent atrial fibrosis. In certain embodiments, said composition may reduce atrial fibrosis by decreasing atrial collagen level when compared to an untreated subject, wherein atrial collagen level may be quantified using hydroxyproline comprising a decrease in hydroxyproline level by about 1% to about 100% when compared to an untreated subject.

In another embodiment of any of the above compositions, said extracellular vesicles may reduce atrial fibroblast proliferation. In certain embodiments, said reduction of atrial fibroblast proliferation may be a decrease in relative atrial fibroblast cell number over time when compared to an untreated subject comprising a decrease in relative atrial fibroblast cell number over time by about 1% to about 100% when compared to the untreated subject. In certain embodiments, reduction of atrial fibroblast proliferation may comprise a decrease in the number of cells in S-phase of the cell cycle when compared to untreated subjects. In other embodiments, the number of cells in S-phase of the cell cycle may be measured using one or more of the following: flow cytometry, western blot, ELISA, bromodeoxyuridine (BrDU), 5-ethynyl-2′deoxyuridine (EdU), phosphorylation of the retinoblastoma protein, cyclin D, cyclin E, cyclin A, cyclin B, any equivalent methods or any combination thereof. In further embodiments, a decrease in the number of cells in S-phase of the cell cycle may comprise about 1% to about 100% when compared to untreated subjects.

In certain embodiments, said reduction of atrial fibroblast proliferation may be an increase in the number of cells in G0 or G1 of the cell cycle when compared to an untreated subject, wherein the number of cells in G0 or G1 of the cell cycle may be measured using one or more of the following: flow cytometry, western blot, ELISA, Ki67, BrDU, EdU, propidium iodide, phosphorylation of the retinoblastoma protein, cyclin D, cyclin E, any equivalent methods or combination thereof, comprising an increase by about 1% to about 100% or more, when compared to an untreated subject.

In certain embodiments, said reduction of atrial fibroblast proliferation may comprise a decrease in the number of cells in G2 or M phase of the cell cycle when compared to untreated subjects, wherein said decrease in the number of cells in G2 or M phase of the cell cycle when compared to untreated subjects is measured using one or more of the following: flow cytometry, western blot, ELISA, phosphorylation of histone H3, propidium iodide, cyclin B, cyclin B, cyclin A, cyclin D, CDK1, CDK2, DAPI. Hoechst, Ki67, any equivalent methods or combination thereof, comprising a decrease by about 1% to about 100% when compared to an untreated subject. In some embodiments, atrial fibroblast proliferation may be measured in vitro. In some embodiments, atrial fibroblast proliferation may be measured in vivo.

In another embodiment of any of the compositions above, reduction of atrial fibroblast proliferation may comprise a decrease in transcription of positive cell cycle regulatory genes when compared to an untreated subject. In another embodiment of any of the compositions above, reduction of atrial fibroblast proliferation may comprise an increase in transcription of negative cell cycle regulatory genes when compared to an untreated subject.

In another embodiment of any of the compositions above, said extracellular vesicles may reduce the relative mass of the atria when compared to an untreated subject, comprising comparing the ratios of atrial mass to total body mass (relative mass=atrial mass/total body mass) between a subject treated with the extracellular vesicles and the untreated subject, said reduction may comprise a relative mass of about 1% to about 100% less when compared to an untreated subject.

In another embodiment of any of the compositions above, said biologically or pharmaceutically acceptable adjuvant or carrier may be appropriate for systemic delivery. In certain embodiments, said biologically or pharmaceutically acceptable adjuvant or carrier may be appropriate for local delivery. In other embodiments, said biologically or pharmaceutically acceptable adjuvant or carrier may be appropriate for intra-myocardial delivery. In other embodiments, said biologically or pharmaceutically acceptable adjuvant or carrier may be appropriate for intra-atrial delivery. In other embodiments, said biologically or pharmaceutically acceptable adjuvant or carrier may be appropriate for catheter delivery.

In another embodiment of any of the compositions above, said extracellular vesicles may improve one or more symptoms including general fatigue, rapid heartbeat, irregular heartbeat, dizziness, shortness of breath, anxiety, syncope, heart failure, neck pounding, weakness, confusion, faintness, sweating, chest pain, chest pressure, chest fluttering, or any combination thereof. In certain embodiments, said extracellular vesicles may reduce the duration of atrial fibrillation, where said reduced duration of atrial fibrillation is determined using electrocardiogram, continuous electrocardiogram monitor, blood pressure machine, Holter monitor, event monitor, blood tests, echocardiogram, stress test, chest x-ray, smart watch, smart ring, any wearable technology capable of determining heart rate, any equivalent techniques or any combination thereof.

In another embodiment of any of the compositions above, said extracellular vesicles may reduce the incidence of atrial fibrillation, where said reduced incidence of atrial fibrillation is determined using electrocardiogram, continuous electrocardiogram monitor, blood pressure machine, Holter monitor, event monitor, blood tests, echocardiogram, stress test, chest x-ray, smart watch, smart ring, any wearable technology capable of determining heart rate, any equivalent techniques or any combination thereof.

In another embodiment of any of the compositions above, wherein said extracellular vesicles may be solubilized by the addition of a detergent, comprising a solution of about 0.01% to about 10.0% Triton™ X-100, wherein solubilisation may be measured by a decrease in the extracellular vesicle particle number compared to a sample not treated with the detergent.

In another embodiment, there is provided herein a method for the treatment or prevention of atrial arrhythmia, wherein the method may comprise administering any of the compositions above to a subject in need thereof.

In another embodiment, there is provided herein a method for the reduction or prevention of atrial fibrosis, wherein the method may comprise administering any of the compositions above to a subject in need thereof.

In another embodiment, there is provided herein a method for the reduction or prevention of atrial inflammation, wherein the method may comprise administering any of the compositions above to a subject in need thereof.

In another embodiment, there is provided herein a method for the reduction or prevention of atrial fibroblast proliferation, wherein the method may comprise administering any of the compositions above to a subject in need thereof.

In another embodiment of any of the above methods, said atrial arrhythmia may be diagnosed with one or more techniques including electrocardiogram, continuous electrocardiogram monitor, blood pressure machine, Holter monitor, event monitor, blood tests, echocardiogram, stress test, chest x-ray, smart watch, smart ring, any wearable technology capable of determining heart rate, any equivalent techniques or any combination thereof. In another embodiment, said subject with atrial arrhythmia may have one or more symptoms including general fatigue, rapid heartbeat, irregular heartbeat, dizziness, shortness of breath, anxiety, syncope, heart failure, neck pounding, weakness, confusion, faintness, sweating, chest pain, chest pressure, chest fluttering, or any combination thereof.

In another embodiment of any of the above methods, said extracellular vesicles may be administered systemically. In certain embodiments, said extracellular vesicles may be administered locally. In certain embodiments, said extracellular vesicles may be administered intra-myocardially. In certain embodiments, said extracellular vesicles may be administered intra-atrially. In another embodiment, local administration may comprise injection. In certain embodiments, injection may comprise 1 to 500 injections. In further embodiments, local administration comprising injection may comprise an injection about every 0.10 cm² to about every 10.00 cm², wherein surface area may be based on the tissue being injected, for example, the surface area of the atria of the heart, the surface area of the left atrium of the heart, the surface area of the right atrium, the surface area of the ventricles of the heart, the surface area of the left ventricle of the heart, the surface area of the right ventricle of the heart, or the surface area of the entire heart. In a preferred embodiment, intra-atrially administration may comprise injection, which may further comprise 1 to 500 injections, wherein intra-atrially administration may comprise injection about every 0.10 cm² to about every 10.0 cm² of atrial surface area.

In another embodiment of any of the above methods, said extracellular vesicles may be administered by catheter. In certain embodiments, said extracellular vesicles may be administered by catheter in the small vessels that perfuse the atria. In certain embodiments, said extracellular vesicles may be administered using a biomaterial that surrounds the atria.

In another embodiment of any of the above methods, said extracellular vesicles may reduce atrial inflammation. In certain embodiments, said reduction of atrial inflammation may be quantified histologically, comprising a reduced number of inflammatory cells in the atria of the heart when compared to an untreated subject. In certain embodiments, the number of inflammatory cells in the atria is quantified using one or more markers may comprise: hematoxylin and eosin staining, activated T lymphocytes, macrophage infiltration, macrophage polarization, mast cells, neutrophils or any combination thereof. In certain embodiments, said histological reduction in the number of inflammatory cells in the atria may comprise a decrease in the inflammatory cells in the atria ranging from about 1% to about 100% when compared to an untreated subject. In certain embodiments, reduction in atrial inflammation may be a reduced level of cytokines in the heart when compared to an untreated subject, wherein one or more of said cytokines comprising: IL-10, IL-2, IL-3, IL-4, IL-5, IL-6, IL-7, IL-8, IL-9, IL-10, IL-12, IL-11, IL-13, IL-16, IL-17, IL-18, G-CSF, GM-CSF, MCP-1, PDGF-AB, TNF-α, any type I IFN, any type II IFN, any type III IFN or any combination thereof. In certain embodiments, said reduction in cytokine level may be quantified using multiplex Luminex-based assay or equivalent technique, and may comprise a decrease in one or more of the cytokines by about 1% to about 100% when compared to an untreated subject.

In another embodiment of any of the above methods, said extracellular vesicles may reduce atrial fibrosis. In certain embodiments, said extracellular vesicles may reduce atrial fibrosis by decreasing atrial collagen, wherein atrial collagen level may be quantified using hydroxyproline, any equivalent or any combination thereof, comprising a decrease in hydroxyproline level of about 1% to about 100% when compared to an untreated subject. It is contemplated, that in certain embodiments, atrial fibrosis may be quantified by comparing the ratios of atrial mass to total body mass (relative mass=atrial mass/total body mass) between a subject treated with the extracellular vesicles and an untreated subject, wherein greater values indicate increased fibrosis and lesser values indicate decreased fibrosis In another embodiment of any of the above methods, said extracellular vesicles may reduce atrial fibroblast proliferation. In certain embodiments, said reduction of atrial fibroblast proliferation may comprise a decrease in the number of cells in S-phase of the cell cycle when compared to untreated subjects, wherein the number of cells in S-phase of the cell cycle may be measured using one or more of the following: flow cytometry, western blot, ELISA, bromodeoxyuridine (BrDU), 5-ethynyl-2′deoxyuridine (EdU), any nucleoside analogue, phosphorylation of the retinoblastoma protein, cyclin D, cyclin E, cyclin A, cyclin B, any equivalent methods or any combination thereof, comprising a decrease in the number of cells in S-phase of the cell cycle by about 1% to about 100% when compared to untreated subjects. In certain embodiments, said reduction of atrial fibroblast proliferation may comprise a decrease in the number of cells in S-phase of the cell cycle when compared to untreated subjects is measured using flow cytometry or equivalent methods. In certain other embodiments, said reduction of atrial fibroblast proliferation may be an increase in number of cells in G0 or G1 of the cell cycle when compared to an untreated subject, wherein the number of cells in G0 or G1 of the cell cycle may be measured using one or more of the following: flow cytometry, western blot, ELISA, Ki67, BrDU, EdU, propidium iodide, phosphorylation of the retinoblastoma protein, cyclin D, cyclin E, any equivalent methods or any combination thereof, wherein said increase in the number of cells in G0 or G1 of the cell cycle may comprise an increase by about 1% to about 100% or more, when compared to an untreated subject. In certain other embodiments, reduction of atrial fibroblast proliferation may comprise a decrease in the number of cells in G2 or M phase of the cell cycle when compared to untreated subjects, wherein said decrease in the number of cells in G2 or M phase of the cell cycle when compared to untreated subjects may be measured using one or more of the following: flow cytometry, western blot, ELISA, phosphorylation of histone H3, propidium iodide, cyclin B, cyclin B, cyclin A, cyclin D, CDK1, CDK2, DAPI. Hoechst, Ki67, any equivalent methods or combination thereof, comprising a decrease by about 1% to about 100% when compared to an untreated subject.

In another embodiment of any of the methods above, reduction of atrial fibroblast proliferation may comprise a decrease in transcription of positive cell cycle regulatory genes when compared to an untreated subject. In another embodiment of any of the methods above, reduction of atrial fibroblast proliferation may comprise an increase in transcription of negative cell cycle regulatory genes when compared to an untreated subject.

In another embodiment of any of the methods above, said extracellular vesicles may improve one or more symptoms including general fatigue, rapid heartbeat, irregular heartbeat, dizziness, shortness of breath, anxiety, weakness, confusion, faintness, sweating, chest pain, chest pressure, chest fluttering or any combination thereof. In certain embodiments, said extracellular vesicles may reduce the duration of atrial fibrillation, where said reduced duration of atrial fibrillation may be determined using electrocardiogram, continuous electrocardiogram monitor, blood pressure machine, Holter monitor, event monitor, blood tests, echocardiogram, stress test, chest x-ray, smart watch, smart ring, any wearable technology capable of determining heart rate, any equivalent techniques or any combination thereof. In certain embodiments, said extracellular vesicles may reduce the incidence of atrial fibrillation where said reduced incidence of atrial fibrillation may be determined using electrocardiogram, continuous electrocardiogram monitor, blood pressure machine, Holter monitor, event monitor, blood tests, echocardiogram, stress test, chest x-ray, smart watch, smart ring, any wearable technology capable of determining heart rate, any equivalent techniques or any combination thereof.

In yet another embodiment there is provided herein a method, use, cell, kit, or composition as described anywhere herein.

### BRIEF DESCRIPTION OF DRAWINGS

FIG. 1 shows the experimental design for derivation of extracellular vesicles (EVs) from heart explant derived cells, use of said extracellular vesicles in a rodent model of atrial arrhythmia and analysis of the impact of the EVs on atrial inflammation, atrial fibrosis and atrial fibrillation.

FIG. 2 shows an exemplary proteomic array demonstrating presence of transmembrane markers (CD63, CD81, FLOT1, ICAM1, EpCam) and cytosolic markers (ALIX, ANXA5 and TSG101) indicative of EV identity while lacking cellular contaminants (GM130).

FIG. 3A-C shows the diameter and abundance of extracellular vesicles derived from human heart cells. (A) Flow cytometry demonstrating the relationship between EV size and surface marker expression (n=3 biological replicates). (B) Quantification of concentration (EVs/mL) of extracellular vesicles expressing the surface markers CD81, CD9 or CD63 (n=3 biological replicates). One-way ANOVA with individual-mean comparisons by Bonferroni's multiple two-tailed comparisons test. (C) Quantification of diameter (nm) of extracellular vesicles expressing the surface markers CD81, CD9 or CD63 (n=3 biological replicates). One-way ANOVA with individual-mean comparisons by Bonferroni's multiple two-tailed comparisons test.

FIG. 4 shows representative flow cytometry images showing the relationship between EV diameter and surface marker expression. EVs within a range of 100-500 nm in diameter are gated. Fluorescence intensity is quantified using standard units known as Molecules of Equivalent Soluble Fluorochrome (MESF).

FIG. 5A-B shows miRNA abundance within extracellular vesicles derived from human heart cells. (A) The miRNA content within EDC EVs was profiled using multiplex fluorescent oligonucleotide-based miRNA detection. Relative abundance of microRNA transcripts within EDC EVs is shown (n=3 biological replicates). (B) Pathway analysis of microRNA transcripts within EDC EVs is shown. Pathway analysis terms were extracted from the Reactome database for network analysis. EV, extracellular vesicles; FDR, false discovery rate; miR, microRNA.

FIG. 6 shows the relative abundance of proteins within extracellular vesicles derived from human heart cells (n=3 biological replicates).

FIG. 7 shows human heart explant-derived cells cultured under serum-free xenogen-free culture conditions within a clinical cell manufacturing facility to produce extracellular vesicles that contain anti-fibrotic/anti-inflammatory proteins. An enrichment map of the EDV EV proteome demonstrating the relationship between biological pathways that are expressed more than would be expected by chance is shown. Red nodes indicate pathways that are predicted to have a positive effect while blue nodes indicate pathways predicted to have a negative effect. ECM, extracellular matrix; MMP2, matrix metalloprotease 2.

FIG. 8 shows flow cytometry quantification of in vitro uptake of 1,1′-Dioctadecyl-3,3,3′,3′-Tetramethylindocarbocyanine Perchlorate (Dil) labelled EVs by rat atrial fibroblasts, cardiomyocytes and human umbilical vein endothelial cells. Cardiomyocytes, red line with squares; endothelial cells, black line with triangles; fibroblasts, blue line with circles.

FIG. 9 shows a study schematic demonstrating the study design, numbers of animals included, group allocations and outcomes measured for determining the effect of human heart cell derived extracellular vesicles in a rodent model of atrial arrhythmia.

FIG. 10 shows representative tracings from an electrocardiogram (ECG) showing the effect of EV treatment on talc-treated animals after burst pacing. EV, extracellular vesicles; veh, vehicle

FIG. 11 shows the effect of EV treatment on the probability of inducing atrial fibrillation (AF) after burst pacing. Superimposed numbers indicate the absolute number of female (lower grey bar+number) or male (upper black bar+number) animals that went into AF. Logistic regression using a generalized linear model with binomial probability distribution and the logit link function was performed. AF, atrial fibrillation; EV, extracellular vesicles; veh, vehicle.

FIG. 12 shows the effect of EV treatment on the mean duration of AF episodes (n=2-25). AF, atrial fibrillation; EV, extracellular vesicles; veh, vehicle.

FIG. 13A-B shows the effect of EV treatment on atrial fibrosis and structure. (A) Atrial fibrosis was measured using hydroxyproline for the indicated experimental groups (n=10). One-way ANOVA with individual-mean comparisons by Bonferroni's multiple two-tailed comparisons test. (B) Relative atrial mass was measured using a ratio of atrial weight to body weight for the indicated experimental groups (n=10-15). One-way ANOVA with individual-mean comparisons by Bonferroni's multiple two-tailed comparisons test. AF, atrial fibrillation; EV, extracellular vesicles; veh, vehicle.

FIG. 14A-B shows the effect of human heart extracellular vesicles (EVs) on inflammation. (A) Representative images of atrial tissue sections after hematoxylin and eosin staining showing the effect of talc and EVs on inflammatory cell infiltration. (B) Quantitative analysis of hematoxylin and eosin staining showing the effect of talc and EVs on inflammatory cell infiltration (n=5 biological replicates).

FIG. 15 shows the effect of talc and EVs on ILβ, IL2, IL6, IL18, monocyte chemoattractant protein 1 (MCP1), platelet-derived growth factor AB (PDGF-AB) and tumor necrosis factor alpha (TNFα) level (n=5 biological replicates). IL, interleukin.

FIG. 16 shows the effect of human heart extracellular vesicles (EVs) on atrial fibroblast proliferation. (A) Shows the effect of heart explant-derived cell (EDC) EVs on relative cell number. (B) Shows the effect of heart explant-derived cell (EDC) EVs on population doubling time.

FIG. 17 shows the effect of heart explant-derived cell (EDC) extracellular vesicles (EVs) on atrial fibroblast proliferation. Representative images showing 5-ethynyl-2′-deoxyuridine (EDU, shown in green) and 4′,6-diamidino-2-phenylindole (DAPI, shown in blue) atrial fibroblasts at baseline and after treatment with interleukin 6 (IL6), transforming growth factor beta 1 (TGFβ1) and/or EDC EVs. Scale bar 200 μM.

FIG. 18 shows quantification of the nuclear incorporation of the thymidine analogue 5-ethynyl-2′-deoxyuridine (EdU) within atrial fibroblasts at baseline and after treatment with interleukin 6 (IL6), transforming growth factor beta 1 (TGFβ1), and/or EDC EVs (n=5 biological replicates).

FIG. 19 shows cell cycle analysis showing the effect of anaphase-promoting complex (APC), IL6, TGFβ1 and/or EDC EVs on the proportion of atrial fibroblasts in the G0/G1 or S and G2/M phases of the cell cycle (n=5 biological replicates). (A) Shows pie charts illustrating the proportion of cells in G0/G1 (Blue), S (Red) or G2/M (Grey) phases of the cell cycle. (B) Shows the percentage of cells in G0/G1 phases of the cell cycle. (C) Shows the percentage of cells in S+G2/M of the cell cycle.

FIG. 20 shows the effect of IL6, TGFβ1 and/or EDC EVs on proteomic expression of cyclins that control progression of atrial fibroblasts through the cell cycle (n=3 biological replicates). Cyclin A2, Cyclin B1, Cyclin D and Cyclin E protein level was determined using enzyme-linked immunosorbent assay (ELISA).

FIG. 21 shows representative images of flow cytometry demonstrating EV depletion after solubilisation. The addition of 0.1% Triton X¬100 solubilizes CD63 stained EVs. Number inside the gates represent percentage of gated population within all particles detected. PE MSEF, Phycoerythrin Molecules of Equivalent Soluble Fluorochrome.

FIG. 22 shows the in vivo study conduct and group sizes. It was assumed the incidence of atrial fibrillation (AF) would be 0.6 after talc treatment, and that extracellular vesicle (EV) treatment would reduce AF incidence to 0.2. Sex was assumed not to alter the incidence of inducible AF. Based on these assumptions, group sample sizes of 34 rats (17 female+17 male) would achieve an 83% power to detect superiority using a two-sided Mann-Whitney test (probability of a false positive result (alpha error)=0.05). Animals that died because of anesthetic overdose prior to the second procedure were not included in the analysis. An allocation error resulted in 1 additional female rat receiving EVs. To avoid compromising the data, the animal was included in the analysis. Given that recipient sex did not influence occurrence of the primary outcome (i.e., AF inducibility), secondary outcomes (e.g., histology, fibrosis, etc.) were not analyzed separately for recipient sex. EPS, electrophysiological study, EV, extracellular vesicle, veh, vehicle.

FIG. 23 shows the effect of human heart derived EVs on inflammasome activation using a luciferase reporter for caspase-1 activation. THP-1 cells were differentiated for 3 days with phorbol 12-myristate 13-acetate (PMA). Medium was replaced and EVs were added followed by lipopolysaccharide (LPS). Half of the medium was removed after 3 hours and luciferase emission was tested with vehicle or the caspase inhibitor, YVAD-CHO. RLU, relative light units.

FIG. 24 shows the effect of the dosage of human heart derived EVs on atrial fibrillation, fibrosis and inflammation. (A) Probability of atrial fibrillation (>0.5 seconds) was measured after burst pacing with the indicated EV concentrations. Logistic regression using a generalized linear model with binomial probability distribution and the logit link function was performed. (B) Atrial fibrosis was measured using hydroxyproline for the indicated experimental groups. ANOVA with Tukey's multiple comparison test. (C) Inflammation of atria treated with human heart derived EVs as indicated was measured using enzyme-linked immunosorbent assay. ANOVA followed by Tukey's multiple comparison test. IL, interleukin; MCP, monocyte chemoattractant protein; PDGF, platelet-derived growth factor.

FIG. 25 shows the effect of human heart derived EVs and the effect of colchicine on atrial fibrillation, fibrosis and inflammation. (A) Probability of atrial fibrillation (>0.5 seconds) was measured after burst pacing with the indicated treatment. Logistic regression using a generalized linear model with binomial probability distribution and the logit link function was performed. (B) Atrial fibrosis was measured using hydroxyproline for the indicated experimental groups. ANOVA with Tukey's multiple comparison test. (C) Inflammation of atria treated as indicated was measured using enzyme-linked immunosorbent assay. ANOVA followed by Tukey's multiple comparison test. IL, interleukin; MCP, monocyte chemoattractant protein.

FIG. 26 shows the effect of human heart derived EVs on infarction induced atrial fibrillation. Atrial fibrillation (>0.5 seconds) was measured for the indicated groups. Logistic regression using a generalized linear model with binomial probability distribution and the logit link function was performed.

FIG. 27 shows the uptake of human heart derived EVs. Flow cytometry showing uptake of labelled EVs by macrophages (Raw 264.7), primary cultured rat atrial fibroblasts (AF), human umbilical vein endothelial cells (HUVECs), primary human pulmonary microvascular endothelial cells (HPMEC), neo-natal rat ventricular myocytes (NRVMs), hepatic G2 cells (HepG2), and human embryonic kidney cells (HEK);

FIG. 28 shows the experimental design for the derivation of extracellular vesicles (EVs) from the indicated tissues, and the steps for miRNA and protein profiling from the EVs therefrom;

FIG. 29A-B shows (A) exemplary plots of tracking analysis of EVs and (B) the concentration and size of EVs isolated from the indicated cell types;

FIG. 30 shows an exemplary proteomic array demonstrating presence of transmembrane markers (CD63, CD81, FLOT1, ICAM1, EpCam) and cytosolic markers (ALIX, ANXA5 and TSG10) indicative of EV identity while lacking cellular contaminants (GM130);

FIG. 31A-C shows EV miRNA cargo profiles across tissues. (A) Venn diagram showing distinct number of miRNAs identified in three cell types. (B) Venn diagram showing the distinct highly abundant (above 99^{th} percentile) miRNAs between cell types. (C) Rank order plots showing distinct number of miRNAs identified in three cell types. The highly abundant (above 99th percentile) miRNAs are highlighted on the rank order plots. Data are presented as mean values, n=3 biological replicates; each circle on the rank order plots represents one distinct miRNA. miRNA: microRNA, BM-MSC: Bone marrow derived mesenchymal stromal cells, HDC: Heart derived cells, UC-MSC: Umbilical cord derived mesenchymal stromal cells.

FIG. 32A-C shows EV protein cargo profiles across tissues. (A) Venn diagram showing distinct number of proteins identified in three cell types. (B) Venn diagram showing the distinct highly abundant (above 99^{th} percentile) proteins between cell types. (C) Rank order plots showing distinct number of proteins identified in three cell types. The highly abundant (above 99th percentile) proteins are highlighted on the rank order plots. Data are presented as mean values, n=3 biological replicates; each circle on the rank order plots represents one distinct protein. LFQ: label-free quantitation; BM-MSC: Bone marrow derived mesenchymal stromal cells, HDC: Heart derived cells, UC-MSC: Umbilical cord derived mesenchymal stromal cells.

FIG. 33 shows differential expression analysis of EV miRNA cargo of HDC vs BM MSC. miRNAs were considered differentially expressed with a log 2 fold change ≥ or ≤1.5 and p-value<0.05. Volcano plot showing 20 down regulated and 36 upregulated miRNA transcripts and heatmap showing the significant differentially expressed miRNAs in HDC vs. BM-MSC EVs. The list of differentially expressed miRNAs is provided in TABLE 3. n=3 biological replicates. BM-MSC: Bone marrow derived mesenchymal stromal cells, HDC: Heart derived cells;

FIG. 34 shows differential expression analysis of EV miRNA cargo of BM MSC vs UC MSC. miRNAs were considered differentially expressed with a log 2 fold change ≥ or ≤1.5 and p-value <0.05. Volcano plot showing 12 down regulated and 164 upregulated miRNA transcripts and heatmap showing the significant differentially expressed miRNAs in BM-MSC vs. UC-MSC EVs. The list of differentially expressed miRNAs is provided in the TABLE 4. n=3 biological replicates. BM-MSC: Bone marrow derived mesenchymal stromal cells, UC-MSC: Umbilical cord derived mesenchymal stromal cells.

FIG. 35 shows differential expression analysis of EV miRNA cargo of HDC vs UC MSC. miRNAs were considered differentially expressed with a log 2 fold change ≥ or ≤1.5 and p-value <0.05. Volcano plot showing 15 down regulated and 299 upregulated miRNA transcripts and heatmap showing the significant differentially expressed miRNAs in HDC vs. UC-MSC EVs. The list of differentially expressed miRNAs is provided in the TABLE 5. n=3 biological replicates. HDC: Heart derived cells, UC-MSC: Umbilical cord derived mesenchymal stromal cells;

FIG. 36 shows differential protein expression analysis of EV cargo in HDC vs BM MSC. Proteins identified in at least 2 of 3 replicates were considered for analysis. The proteins were considered differentially expressed with a p-value <0.05. Volcano plot showing 9 down regulated and 14 upregulated proteins and heatmap showing the significant differentially expressed proteins in HDC vs. BM-MSC EVs. The list of differentially expressed proteins is provided in TABLE 6. n=3 biological replicates. BM-MSC: Bone marrow derived mesenchymal stromal cells, HDC: Heart derived cells;

FIG. 37 shows differential protein expression analysis of EV cargo in BM MSC vs UC MSC. Proteins identified in at least 2 of 3 replicates were considered for analysis. The proteins were considered differentially expressed with a p-value <0.05. Volcano plot showing 198 down regulated and 107 upregulated proteins and heatmap showing the significant differentially expressed proteins in BM-MSC vs. UC-MSC EVs. The list of differentially expressed proteins is provided in TABLE 7. n=3 biological replicates. BM-MSC: Bone marrow derived mesenchymal stromal cells, UC-MSC: Umbilical cord derived mesenchymal stromal cells.

FIG. 38 shows differential protein expression analysis of EV cargo in HDC vs UC MSC. Proteins identified in at least 2 of 3 replicates were considered for analysis. The proteins were considered differentially expressed with a p-value <0.05. Volcano plot showing 141 down regulated and 119 upregulated proteins and heatmap showing the significant differentially expressed proteins in HDC vs. UC-MSC EVs. The list of differentially expressed proteins is provided in TABLE 8. n=3 biological replicates. HDC: Heart derived cells, UC-MSC: Umbilical cord derived mesenchymal stromal cells.

FIG. 39A-B shows a functional enrichment analysis of differentially expressed EV miRNA cargo of HDC vs BM MSC. (A) The top 10 significantly enriched biological functions and Gene ontology biological processes of all differentially expressed miRNAs (DEM)s between HDC vs BM MSC. (B) The top 10 significantly enriched biological functions of enriched miRNAs in HDC in comparison to BM MSC, and BM MSC in comparison to HDC. n=3 biological replicates. Activ: Activity, BM-MSC: Bone marrow derived mesenchymal stromal cells, Dev: Development, Diff: Differentiation, HDC: Heart derived cells, MET: Mesenchymal-epithelial transition, UC-MSC: Umbilical cord derived mesenchymal stromal cells.

FIG. 40A-B shows a functional enrichment analysis of differentially expressed EV miRNA cargo of HDC vs BM MSC. (A) The top 10 significantly enriched transcription factor & tissue specificity of all differentially expressed miRNAs (DEM)s between HDC vs BM MSC. (B) The top 10 significantly enriched transcription factor & tissue specificity of enriched miRNAs in HDC in comparison to BM MSC, and BM MSC in comparison to HDC. n=3 biological replicates. In a case the analysis yielded less than 10 enriched terms, all the terms are shown the in graphs. BM-MSC: Bone marrow derived mesenchymal stromal cells, HDC: Heart derived cells, UC-MSC: Umbilical cord derived mesenchymal stromal cells.

FIG. 41A-B shows a functional enrichment analysis of differentially expressed EV miRNA cargo of BM MSC vs UC MSC. (A) The top 10 significantly enriched biological functions and Gene ontology biological processes of all differentially expressed miRNAs (DEM)s among BM MSC vs UC MSC. (B) The top 10 significantly enriched biological functions of enriched miRNAs in BM USC in comparison to UC MSC, and UC MSC in comparison to BM MSC. n=3 biological replicates. Activ: Activity, BM-MSC: Bone marrow derived mesenchymal stromal cells, Dev: Development, Diff: Differentiation, HDC: Heart derived cells, MET: Mesenchymal-epithelial transition, UC-MSC: Umbilical cord derived mesenchymal stromal cells.

FIG. 42A-B shows a functional enrichment analysis of differentially expressed EV miRNA cargo of BM MSC vs UC MSC. (A) The top 10 significantly enriched transcription factor & tissue specificity of all differentially expressed miRNAs (DEM)s between BM MSC vs UC MSC. (B) The top 10 significantly enriched transcription factor & tissue specificity of enriched miRNAs in BM MSC in comparison to UC MSC, and UC MSC in comparison to BM MSC. n=3 biological replicates. BM-MSC: Bone marrow derived mesenchymal stromal cells, HDC: Heart derived cells, UC-MSC: Umbilical cord derived mesenchymal stromal cells.

FIG. 43A-B shows a functional enrichment analysis of EV differentially expressed miRNA cargo of HDC vs UC MSC. (A) The top 10 significantly enriched biological functions and Gene ontology biological processes of all differentially expressed miRNAs (DEM)s among HDC vs UC MSC. (B) The top 10 significantly enriched biological functions of enriched miRNAs in HDC in comparison to UC MSC, and UC MSC in comparison to HDC. n=3 biological replicates. Activ: Activity, BM-MSC: Bone marrow derived mesenchymal stromal cells, Dev: Development, Diff: Differentiation, HDC: Heart derived cells, MET: Mesenchymal-epithelial transition, UC-MSC: Umbilical cord derived mesenchymal stromal cells.

FIG. 44A-B shows a functional enrichment analysis of differentially expressed EV miRNA cargo of HDC vs UC MSC. (A) The top 10 significantly enriched transcription factor & tissue specificity of all differentially expressed miRNAs (DEM)s between HDC vs UC MSC. (B) The top 10 significantly enriched transcription factor & tissue specificity of enriched miRNAs in HDC in comparison to UC MSC, or UC MSC in comparison to HDC. n=3 biological replicates. BM-MSC: Bone marrow derived mesenchymal stromal cells, HDC: Heart derived cells, UC-MSC: Umbilical cord derived mesenchymal stromal cells;

FIG. 45A-B shows a functional enrichment analysis of differentially expressed EV protein cargo of HDC vs BM MSC. (A) The top 10 significantly enriched molecular functions of all differentially expressed proteins (DEP)s among HDC vs BM MSC. (B) The top 10 significantly enriched molecular functions of enriched proteins in HDC in comparison to BM MSC, and BM MSC in comparison to HDC. n=3 biological replicates. BM-MSC: Bone marrow derived mesenchymal stromal cells, ECM: Extracellular matrix, GDP: Guanosine diphosphate, GTP: Guanosine triphosphate, HDC: Heart derived cells, LDL: Low-density lipoprotein, Macromol.cmpx: Macromolecule complex, MHC: Myosin heavy chain, Protein. Ppase: Protein phosphatase, UC-MSC: Umbilical cord derived mesenchymal stromal cells; cellular component (CC); biological process (BP);

FIG. 46A-B shows a functional enrichment analysis of differentially expressed EV protein cargo of HDC vs BM MSC. (A) The top 10 significantly enriched biological process (BP) and cellular component (CC) of all differentially expressed proteins (DEP)s among HDC vs BM MSC. (B) The top 10 significantly enriched biological process (BP) and cellular component (CC) of enriched proteins in HDC in comparison to BM MSC, and BM MSC in comparison to HDC. n=3 biological replicates. BM-MSC: Bone marrow derived mesenchymal stromal cells, ER: Endoplasmic reticulum, diff: Differentiation, ECM: Extracellular matrix, HDC: Heart derived cells, UC-MSC: Umbilical cord derived mesenchymal stromal cells;

FIG. 47A-B shows a functional enrichment analysis of differentially expressed EV protein cargo of BM MSC vs UC MSC. (A) The top 10 significantly enriched molecular functions of all differentially expressed proteins (DEP)s among BM MSC vs UC MSC. (B) The top 10 significantly enriched molecular functions of enriched proteins in BM MSC in comparison to UC MSC, and UC MSC in comparison to BM MSC. n=3 biological replicates. BM-MSC: Bone marrow derived mesenchymal stromal cells, ECM: Extracellular matrix, GDP: Guanosine diphosphate, GTP: Guanosine triphosphate, HDC: Heart derived cells, LDL: Low-density lipoprotein, Macromol.cmpx: Macromolecule complex, MHC: Myosin heavy chain, Protein. Ppase: Protein phosphatase, UC-MSC: Umbilical cord derived mesenchymal stromal cells.

FIG. 48A-B shows a functional enrichment analysis of differentially expressed EV protein cargo of BM MSC vs UC MSC. (A) The top 10 significantly enriched biological process (BP) and cellular component (CC) of all differentially expressed proteins (DEP)s among BM MSC vs UC MSC. (B) The top 10 significantly enriched biological process (BP) and cellular component (CC) of enriched proteins in BM MSC in comparison to UC MSC, and UC MSC in comparison to BM MSC. n=3 biological replicates. BM-MSC: Bone marrow derived mesenchymal stromal cells, ER: Endoplasmic reticulum, diff: Differentiation, ECM: Extracellular matrix, HDC: Heart derived cells, UC-MSC: Umbilical cord derived mesenchymal stromal cells FIG. 49A-B shows a functional enrichment analysis of differentially expressed EV protein cargo of HDC vs UC MSC. (A The top 10 significantly enriched molecular functions of all differentially expressed proteins (DEP)s among HDC vs UC MSC. (B) The top 10 significantly enriched molecular functions of enriched proteins in HDC in comparison to UC MSC, and UC MSC in comparison to HDC. n=3 biological replicates. BM-MSC: Bone marrow derived mesenchymal stromal cells, ECM: Extracellular matrix, GDP: Guanosine diphosphate, GTP: Guanosine triphosphate, HDC: Heart derived cells, LDL: Low-density lipoprotein, Macromol.cmpx: Macromolecule complex, MHC: Myosin heavy chain, Protein. Ppase: Protein phosphatase, UC-MSC: Umbilical cord derived mesenchymal stromal cells.

FIG. 50A-B shows a functional enrichment analysis of differentially expressed EV protein cargo of HDC vs UC MSC. (A) The top 10 significantly enriched biological process (BP) and cellular component (CC) of all differentially expressed proteins (DEP)s among HDC vs UC MSC. (B) The top 10 significantly enriched biological process (BP) and cellular component (CC) of enriched proteins in HDC in comparison to UC MSC, and UC MSC in comparison to HDC. n=3 biological replicates. BM-MSC: Bone marrow derived mesenchymal stromal cells, ER: Endoplasmic reticulum, diff: Differentiation, ECM: Extracellular matrix, HDC: Heart derived cells, UC-MSC: Umbilical cord derived mesenchymal stromal cells.

FIG. 51 shows a protein-protein interaction network and GO-BP (Gene Ontology-Biological Process) analysis of differentially expressed proteins in EVs from BM MSC and USC MSC differentially expressed proteins (DEP)s. Significant clusters from the PPI network and enriched GO-BP terms of BM-MSC vs UC-MSC DEPs. The diamonds indicate nodes, gray lines indicate edges and the circles indicate hub node genes.

FIG. 52 shows a protein-protein interaction network and GO-BP (Gene Ontology-Biological Process) analysis of differentially expressed proteins in EVs from HDC vs UC MSC differentially expressed proteins (DEP)s. Significant clusters from the PPI network and enriched GO-BP terms of HDC vs UC-MSC DEPs. The diamonds indicate nodes, gray lines indicate edges and the circles indicate hub node genes.

FIG. 53A-B show KEGG (Kyoto Encyclopedia of Genes and Genomes) functional enrichment analysis on all differentially expressed proteins (DEPs) or up- or down-regulated proteins between HDC vs BM MSC EVs. (A) The top 10 significantly enriched KEGG pathways of all differentially expressed proteins among HDC vs BM MSC. (B) The top 10 significantly KEGG pathways of enriched proteins in HDC vs BM MSC and BM MSC vs HDC. n=3 biological replicates. In a case the analysis yielded less than 10 enriched terms, all the terms are shown the in graphs. BM-MSC: Bone marrow derived mesenchymal stromal cells, HDC: Heart derived cells;

FIG. 54A-B show KEGG (Kyoto Encyclopedia of Genes and Genomes) functional enrichment analysis on all differentially expressed proteins (DEPs) or up- or down-regulated proteins between BM MSC vs UC MSC EVs (A) The top 10 significantly enriched KEGG pathways of all DEPs UC MSC vs BM MSC. (B) The top 10 significantly KEGG pathways of enriched proteins in UC MSC vs BM MSC and BM MSC vs UC MSC. n=3 biological replicates. BM-MSC: Bone marrow derived mesenchymal stromal cells, E.Coli: Escherichia Coli, UC-MSC: Umbilical cord derived mesenchymal stromal cells;

FIG. 55A-B show KEGG (Kyoto Encyclopedia of Genes and Genomes) functional enrichment analysis on all differentially expressed proteins (DEPs) or up- or down-regulated proteins between HDC and UC MSC EVs. (A) The top 10 significantly enriched KEGG pathways of all DEPs among UC MSC vs HDC. (B) The top 10 significantly KEGG pathways of enriched proteins in UC MSC vs HDC and HDC vs UC MSC. n=3 biological replicates. ECM: Extracellular matrix, GDP: Guanosine diphosphate, GTP: Guanosine triphosphate, HDC: Heart derived cells, UC-MSC: Umbilical cord derived mesenchymal stromal cells;

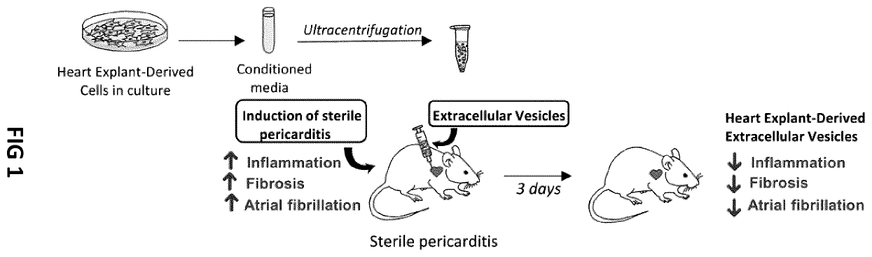

These and other features of the present invention will be further understood with reference to the following description and accompanying drawings, wherein:

### DETAILED DESCRIPTION

Current drug and surgical treatments for atrial arrhythmias can be limited by serious side effects and limited effectiveness. Current drug approaches target heart rate, which is a single mechanism of atrial arrhythmia, however, atrial arrhythmias are considered to be caused by multiple mechanisms including inflammation, fibrosis and fibroblast proliferation. Identifying compositions that target multiple mechanisms that may lead to atrial arrhythmia, may improve atrial arrhythmia treatment and prevention.

As used herein, “patient” or “subject” may encompass any vertebrate organism or cell including mammals, but not limited to humans, non-human primates, rats, dogs, pigs and mice. In a preferred embodiment, the mammal may be a human. Further, a patient or subject ‘in need thereof’ may encompass any of the above organisms diagnosed with atrial arrhythmia, experiencing symptoms of atrial arrhythmia, and/or diagnosed or experiencing symptoms associated with a condition benefiting from the administration of a composition of extracellular vesicles described herein. As used herein, “an untreated subject” may encompass any of the above organisms, cells or material, wherein any control measure was performed or administered, including but not limited to sham surgery, unconditioned media only, vehicle, buffer only, untreated and/or any equivalent or appropriate control measure, instead of the composition of extracellular vesicles. In certain embodiments, ‘an untreated subject’ may be a patient or subject prior to administration of a composition of extracellular vesicles described herein.

As used herein, “in vitro” may encompass experimentation or analysis done on any vertebrate cell cultured outside the organism including mammals, but not limited to humans, non-human primates, rats, dogs, pigs and mice.

As used herein, “in vivo” may encompass experimentation or analysis done on any whole vertebrate organism including mammals, but not limited to humans, non-human primates, rats, dogs, pigs and mice.

Herein ‘about 1% to about 100%’ may encompass any values defining a range therein, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

Extracellular vesicles are naturally secreted, replication incompetent particles, enclosed by a lipid-bilayer, which can transfer its constituents to other cells (Murphy, de Jong et al. 2019). Herein, the term ‘constituents’ refers to the molecular contents of the extracellular vesicle, including but not limited to, proteins, lipids, nucleic acids, metabolites, and organelles. Extracellular vesicles may be referred to by the term polydisperse, as they are a population of particles with non-uniform size, shape, and constituents, further described herein. Extracellular vesicle constituents can vary substantially with factors including but not limited to tissue type, cell type, and physiological conditions. While extracellular vesicles are thought to have potential in treating various conditions, it is not known which extracellular vesicle compositions may have this surprising characteristic. As extracellular vesicle constituents and function can vary between similar cell types, even with modest modifications to their derivation, it is important to identify specific cells and conditions for these compositions. An exemplary non-limiting example of extracellular vesicle compositional diversity including, but not limited to, extracellular vesicles derived from heart-explant derived cells prepared under physiological conditions were reported to have an average size of 120 nm, however, when prepared under standard conditions the average extracellular vesicle size was 148 nm (Mount, Kanda et al. 2019). In addition, the same study reported changes to 51 miRNA constituents under physiological conditions compared to standard conditions. Therefore determining an ideal composition of extracellular vesicles may be critical in developing more effective therapies for atrial arrhythmias, as well as other heart and non-heart conditions. In addition, once an effective composition of extracellular vesicles is determined, its properties, including constituents, and size need to be determined.

Described herein are compositions comprising extracellular vesicles isolated from human heart cells, uses and methods thereof. It will be appreciated that embodiments and examples are provided herein for illustrative purposes intended for those skilled in the art, and are not meant to be limiting in any way.

As part of the studies described in detail herein below, results are provided which demonstrate, in what is believed to be for the first time and without wishing to be bound by theory, that extracellular vesicles derived from human heart cells are capable of ameliorating atrial arrhythmia, as well as atrial fibrosis, atrial fibroblast proliferation, and atrial inflammation. These findings characterize the features of extracellular vesicles derived from human heart cells. Specifically, extracellular vesicles constituents, size, and the ability to transfer constituents to human heart cells were tested, as well as their ability to reduce atrial fibrillation, atrial inflammation, atrial fibrosis and fibroblast proliferation.

Thus, herein, the composition of extracellular vesicles and the characteristics of the extracellular vesicles derived from human heart cells were investigated. Given the diverse nature of extracellular vesicles, they provide a useful platform for testing on heart conditions, including atrial arrhythmias. To reduce the possibility of contamination by non-extracellular structures and therapeutically ineffective extracellular vesicles, the properties of extracellular vesicles isolated were first established.

A person of skill in the art will recognize that different methods to derive extracellular vesicles may be possible and said methods may impact aspects of the extracellular vesicles. The skilled person having regard to the teachings herein will be able to select a suitable method for a given application. Such methods including, but not limited to, differential ultra-centrifugation, size exclusion chromatography, density gradient, tangential flow filtration and variations thereon, microfiltration, ion exchange chromatography, immuno-isolation or a combination thereof (as discussed in, for example, (Konoshenko, Lekchnov et al. 2018)). In one embodiment, extracellular vesicles may be derived from human heart cells by differential ultra-centrifugation. In certain embodiments, it is contemplated that extracellular vesicles may be derived from human heart cells by tangential flow filtration. Exemplary experimental methods for such determinations are provided in Examples described below. The skilled person will understand that various changes, alternative techniques, or substitutions may be made to the experimental methods provided in these Examples, and that the following Examples are intended to be non-limiting.

In an embodiment, there is provided herein a composition comprising extracellular vesicles and a biologically or pharmaceutically acceptable adjuvant or carrier, said extracellular vesicles may comprise one or more of the following characteristics:
extracellular vesicles isolated from human heart cells;
a polydisperse population of particles ranging from about 10 to about 1000 nm, or any values defining a range therein, for example, but not limited to about 90 nm to about 500 nm, about 50 nm to about 250 nm, about 95 to about 170 nm, about 25 nm to about 200 nm, about 50 nm to about 750 nm, about 40 nm to about 600 nm and ranges including and/or spanning the aforementioned values;
one or more cytosolic markers defined by ALIX, any ANXA polypeptide, any ACT polypeptide, TSG101, any CHMP polypeptide, PDCD6IP, VPS4A, VPS4B, ARRDC1, any CAV polypeptide, any EHD polypeptide, RHOA, HSPA8, HSPA1A, any actin polypeptide, any tubulin polypeptide, GAPDH, HSP90AB1, ARF6, SDCBP, MAPT, and TSG101, or a combination thereof,
one or more transmembrane markers defined by CD3, CD9, CD14, CD37, CD41, CD45, CD47, CD53, CD55, CD59, CD61, CD63, CD73, CD81, CD82, CD90, any GNA polypeptide, HLA-A, HLA-B, HLA-C, HLA-DR, HLA-DP, HLA-DQ, any H2-A polypeptide H2-K, H2-D, H2-Q, any ITGA polypeptide, any ITGB polypeptide, TFR2, LAMP1, LAMP2, any SDC polypeptide, BSG, ADAM10, SHH, TSPAN8, PECAMI, ERBB2, EPCAM, CDC42a, GYPA, AChE-S, AChE-E, AβA4, APP, ABCC1, FLOT1, FLOT2, ICAM1, EpCam, or a combination thereof,
acetylcholinesterase activity;
substantially lacking or devoid of one or more of the group comprising GM130, APOA1, APOA2, APOB, APOB100, ALB, ribosomal proteins, UMOD and protein/nucleic acid aggregates;
1-1000 unique miRNA transcripts; and
a unit dosage of extracellular vesicles comprising from about 10² to about 10²⁰ particles per millilitre.

In certain embodiments of any of the compositions or methods above, the extracellular vesicles may be a polydisperse population that comprises an average diameter of about 10 nm to about 500 nm, any values defining a range therein, including, for example, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, and 500 nm.

As taught in (Chiang and Chen 2019), extracellular vesicles from a single source are heterogenous and have a distribution of sizes, which characterize the population. A person of skill in the art will recognize that different methods to measure extracellular vesicle size and number may be possible. Such methods including, but not limited to, nanoparticle tracking analysis, high-resolution flow cytometry, standard flow cytometry, resistive pulse sensing, atomic force microscopy, impedance-based detection, laser tweezers Raman spectroscopy, dark field microscopy, electron microscopy, transmission electron microscopy, and cryo-electron microscopy. The skilled person having regard to the teachings herein will be able to select a suitable method for a given application. The person of skill in the art will also recognize that extracellular vesicle number may be indirectly measured with such methods including total protein amount, total lipid amount, total RNA, acetylcholinesterase activity or quantification of specific molecules. In one embodiment, extracellular vesicle particle size and number may be measured using Nanoparticle Tracking Analysis. In certain embodiments, extracellular vesicle particle number may be measured using FluoroCet Exosome Quantitation kit. Exemplary experimental methods for such determinations are provided in Examples described below. The skilled person will understand that various changes, alternative techniques, or substitutions may be made to the experimental methods provided in these Examples, and that the following Examples are intended to be non-limiting.

The mammalian heart is divided into four chambers consisting of two ventricles (lower chambers) and two atria (upper chambers). The heart chambers are composed of five major cell types including cardiac fibroblasts, cardiomyocytes, cardiac precursor cells, smooth muscle cells and endothelial cells. Herein, all said cells may be encompassed by the expression ‘human heart cell’. In some embodiments, the human heart cells may be derived from a heart biopsy. In certain embodiments, the human heart cells may be derived from a heart biopsy containing any combination of myocardial, epicardial or endocardial tissue. In a further embodiment, the heart biopsy may be an atrial appendage. In some embodiments, the human heart cells may be derived from ventricular tissue. In certain embodiments, the human heart cells may be derived from an entire heart. In a preferred embodiment, the heart biopsy may be a left atrial appendage. In a most preferred embodiment, the left atrial appendage biopsy contains cardiomyocytes and cardiac precursor cells. The skilled person will recognize that in certain embodiments, atrial appendages may be surgically removed at the time of open heart surgery, for example, and ventricular or atrial biopsies may be obtained by guiding a catheter into the heart and taking small bites from the heart tissue, for example. The skilled person will be aware of suitable techniques for obtaining a suitable biopsy or appendage. In some embodiments, heart-derived explant cells may be derived from the heart biopsy. The person of skill in the art will further recognize that heart explant derived cells may represent a collection of different cell populations which express markers of endothelial, mesenchymal, and stem cell identity. Without wishing to be bound by theory, such a cell may likely be considered a multipotent cell, or a stem cell, which has been at least partially differentiated to heart tissue.

Cellular immortalization, as described herein and would be recognized by a person of skill in the art, is a process or manipulation to increase the cellular division capacity of cells in culture (in vitro) thus increasing the length of time they can be cultured. In certain embodiments of any of the compositions or methods above, heart-derived explant cells may be immortalized using techniques including, but not limited to, genetic mutation, expression of a viral gene, expression of a viral gene product, expression of a telomerase reverse transcriptase expression, expression of SV40, or equivalent techniques know to a person of skill in the art.

A person of skill in the art will recognize that different methods to generate human heart cells may be possible (as discussed in, for example, (Balafkan, Mostafavi et al. 2020)). The skilled person having regard to the teachings herein will be able to select a suitable cell type for a given application. Such methods may include heart explant-derived stem cells, heart explant-derived stem cells, cardiac explant-derived cells, cardiac explant-derived stem cells, atrial explant-derived stem cells, atrial explant-derived cells, differentiating induced pluripotent stem cells, differentiating embryonic stem cells, differentiating any adult precursor cells, differentiating adipose-derived stem cells, differentiating mesenchymal stem cell, differentiating P19 cells, differentiating C2C12 cells, any cell capable of cardiac differentiation or using a cell or tissue that produces an equivalent composition of extracellular vesicles.

In certain embodiments of any of the compositions or methods above, the human heart cells may be allogenic to the recipient of the composition. In certain other embodiments, the human heart cells may be autologous to the recipient of the composition. In certain embodiments, the human heart cells may be cryopreserved between −1° C. and −200° C., any values defining a range therein, including, for example, −200° C., −199° C., −198° C., −197° C., −196° C., −195° C., −194° C., −193° C., −192° C., −191° C., −190° C., −189° C., −188° C., −187° C., −186° C., −185° C., −184° C., −183° C., −182° C., −181° C., −180° C., −179° C., −178° C., −177° C., −176° C., −175° C., −174° C., −173° C., −172° C., −171° C., −170° C., −169° C., −168° C., −167° C., −166° C., −165° C., −164° C., −163° C., −162° C., −161° C., −160° C., −159° C., −158° C., −157° C., −156° C., −155° C., −154° C., −153° C., −152° C., −151° C., −150° C., −149° C., −148° C., −147° C., −146° C., −145° C., −144° C., −143° C., −142° C., −141° C., −140° C., −139° C., −138° C., −137° C., −136° C., −135° C., −134° C., −133° C., −132° C., −131° C., −130° C., −129° C., −128° C., −127° C., −126° C., −125° C., −124° C., −123° C., −122° C., −121° C., −120° C., −119° C., −118° C., −117° C., −116° C., −115° C., −114° C., −113° C., −112° C., −111° C., −110° C., −109° C., −108° C., −107° C., −106° C., −105° C., −104° C., −103° C., −102° C., −101° C., −100° C., −99° C., −98° C., −97° C., −96° C., −95° C., −94° C., −93° C., −92° C., −91° C., −90° C., −89° C., −88° C., −87° C., −86° C., −85° C., −84° C., −83° C., −82° C., −81° C., −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., and −1° C.

The International Society for Extracellular Vesicles (ISEV) teaches the minimal information for studies of extracellular vesicles (Thery, Witwer et al. 2018). As taught at the time of application, a consensus has not emerged on specific markers of extracellular vesicle subtypes. As such, it is further taught that operational terms for extracellular vesicle subtypes that refer to physical characteristics such as size, biochemical composition such as protein content, conditions of origin or cells of origin are advised. The ISEV further teaches that three categories of broad markers may be analyzed to demonstrate the presence of extracellular vesicles (Categories 1 and 2) and assess their purity from contamination (Category 3). The ISEV also teaches of markers generally restricted to small extracellular vesicles (Category 4) and markers of potential functional activity (Category 5).

Category 1 is taught to contain transmembrane or GPI-anchored protein, as their presence demonstrates the lipid-bilayer structure specific to extracellular vesicles. Said proteins may comprise the following: tetraspanins (CD63, CD81, CD82); other multi-pass membrane proteins (CD47, GNA); MHC class I (HLA-A/B/C, H2-K/D/Q); integrins (ITGA, ITGB); transferrin receptor (TFR2); LAMP1/2, heparan sulfate proteoglycans (SDC); complement-binding proteins (CD55, CD59); MHC class II (HLA-DR/DP/DQ, H2-A); BSG, ADAM10, CD73, SHH, TSPAN, CD37, CD53, CD9, PECAMI, ERBB2, EPCAM, CD90, CD45, CD41, CD42a, ICAM, GYPA, CD14, CD3, AChE-S, AChE-E, A3, APP, and ABCC1.

Category 2 is taught to contain cytosolic proteins, as their presence demonstrates the preparations ability to enclose intracellular material. Said proteins may comprise the following: ESCRT-I/II/III and accessory proteins (TSG101, CHMP): caveolins (CAV); enzymes (GAPDH); actin (ACT), tubulin (TUB); annexins (ANXA); Heat shock proteins (HSC70, HSP70, HSPA1A HSPA8, HSP84, HSP90AB1); PDCD6IP, VPS4A/B; ARRDC1, FLOT1/2, EHD, RHOA, ARF6, SDCBP, and MAPT. It is further taught that such cytosolic protein constituents of extracellular vesicles vary significantly, and therefore extracellular vesicles may contain additional cytosolic proteins.

Category 3 is taught to contain major constituents of non-extracellular vesicle structures often co-isolated with extracellular vesicles. Said proteins may comprise: lipoproteins (APOA1/2, APOB, APOB100, ALB) protein and protein/nucleic acid aggregates, UMOD and ribosomal proteins. It is further taught that evaluating molecules of Category 3 assesses the purity of the extracellular vesicle preparation.

Category 4 is taught to contain larger structures that, due to their size, are substantially lacking or devoid from smaller extracellular vesicles. Said markers may comprise the following: nucleus (including histones, LMNA); mitochondria (including IMMT, CYC1, TOMM20); endoplasmic reticulum (including CANX, HSP90B1, HSPA5); Golgi apparatus (including GM130); autophagosomes (including ATG9A); and cytoskeleton (including ACTN1/4, KRT18). A person of ordinary skill will recognize that organelles and large sub-cellular structures may be identified with other markers. The person of skill in the art will also recognize determination criteria for substantially lacking or devoid based on a given application or methodology.

Category 5 is taught to contain functional components used to determine the mode of association with extracellular vesicles. Said proteins may comprise: cytokines (including IFNG, IL); growth factors (including VEGFA, FGF1/2, PDGF, EGF, TGFB1/2); adhesion and extracellular matrix proteins (including FN1, COL, MFGE8, LGALS3BP, CD5L, AHSG).

A person of skill in the art will recognize that many protein markers are members of families with similar or homologous proteins, which may be capable of performing the same function. Therefore, a person of skill in the art would recognize that protein markers may or may not apply to all proteins across a family or class. As a non-limiting example, ITGB broadly refers to the entire class of integrin beta-sub unit proteins, in humans comprising ITGB1, ITGB2, ITGB3, ITGB4, ITGB5, ITGB6, ITGB7, and ITGB8. A person of skill of the art will recognize that homologues of said proteins may or may not have the same function.

ISEV further teaches that analytical approaches such as Western blots, high resolution flow cytometry, proteomic array or global proteomic analysis using mass spectrometry techniques can be used to identify proteins of Categories 1-5. A person of skill in the art would recognize that additional methods may be used to determine the presence, absence, or level of a given protein in an extracellular vesicle composition. Exemplary experimental methods for such determinations are provided in Examples described below. The skilled person will understand that various changes, alternative techniques, or substitutions may be made to the experimental methods provided in these Examples, and that the following Examples are intended to be non-limiting.

Further, a person of skill in the art would recognize that structures enclosed by a lipid layer, such as the lipid-bilayer of extracellular vesicles, are susceptible to rupture by detergent containing solutions. A person of skill in the art would recognize this as another non-limiting example of determining and/or validating the properties and/or presence of extracellular vesicles, wherein the addition of detergent may decrease the extracellular vesicle particle number compared to a sample not treated with detergent. In another embodiment of any of the compositions above, wherein said extracellular vesicles may be solubilized by the addition of a detergent, comprising a solution of about 0.01% to about 10.0% Triton™ X-100, any values defining a range therein, including, for example, 0.01%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%3, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15% 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, and 10%, wherein solubilisation may be measured by a decrease in the extracellular vesicle particle number compared to a sample not treated with the detergent. Exemplary experimental methods for such determinations are provided in Examples described below. The skilled person will understand that various changes, alternative techniques, or substitutions may be made to the experimental methods provided in these Examples, and that the following Examples are intended to be non-limiting.

The ISEV teaches the non-limiting nature of such foregoing lists, and recommends they be used as a guide to define extracellular vesicles, as not all constituents are present in all populations of vesicles nor are they absent. The ISEV further teaches that extracellular vesicle constituents not listed in any of Categories 1-5 may be present in extracellular vesicles depending on factors including, but not limited to, tissue type, cell type, derivation methodology and physiological conditions. In light of the foregoing and the common general knowledge, a person of skill in the art would recognize that not all markers will be present or absent on extracellular vesicles from a given cell or tissue type, and that said differences in the presence, absence or levels of certain constituents may act as a defining feature of the extracellular vesicles derived. The skilled person having regard to the teachings herein will be able to select a suitable method and threshold for determining ‘substantially lacking or devoid’ and ‘present’ as it pertains to extracellular vesicle constituents for a given application.

It is understood that certain sub-types of ribonucleic acids (RNA), including, but not limited to, micro RNA (miRNA) and messenger RNA (mRNA) make up a portion of the constituents of extracellular vesicles. miRNA function by regulating post-transcriptional gene expression, generally through binding to complementary sequences on mRNA transcripts. The binding of miRNA to a complementary sequence can result in translational repression, as well as mRNA degradation and/or gene silencing. The activity of miRNA may influence the heart in certain disease states, for example, U.S. Pat. No. 9,828,603 teaches that increasing the level of mir-146a decreases the infarct region in mice following myocardial infarction when compared to animals treated with a control mimic miRNA. U.S. Pat. No. 9,828,603 further teaches that increasing the concentration of mir-210 transfected into cardiomyocytes increased their viability 10-fold following exposure to hydrogen peroxide. The miRNA profile of extracellular vesicles may be determined by reverse transcription polymerase chain reaction (qRT-PCR), miRNA microarray, multiplex fluorescent oligonucleotide-based miRNA detection and RNA sequencing. In one embodiment, the miRNA profile of an extracellular vesicle composition derived from human heart cells may be determined with RNA sequencing. In certain embodiments, the extracellular vesicles contain about 1 to about 1000 unique miRNA transcripts, any values defining a range therein, including, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389,390, 391, 392, 393, 394,395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000. Exemplary experimental methods for such determinations are provided in Examples described below. The skilled person will understand that various changes, alternative techniques, or substitutions may be made to the experimental methods provided in these Examples, and that the following Examples are intended to be non-limiting. Herein a unique miRNA transcript refers to a population of miRNA transcribed from a unique genetic locus.

In some embodiments of any of the above compositions or methods, the extracellular vesicles include a variety of biomolecules, such as nucleic acids and proteins. Extracellular vesicles contain different types of RNA molecules, as taught in (Zimta, Sigurjonsson et al. 2020), and different types of deoxyribonucleic acid (DNA) molecules, as taught in (Hur and Lee 2021). In certain embodiments, the extracellular vesicles contain DNA, DNA fragments, DNA plasmids, mRNA, tRNA, snRNA, piRNA, saRNA, miRNA, rRNA, ribozymes, double stranded RNA, other non-coding and coding RNA. In some embodiments, the extracellular vesicles contain non-coding RNAs (ncRNAs), such as, but not limited to, long non-coding RNAs (lncRNAs), microRNAs (miRNAs), long intergenic non-coding RNA (lincRNA), small nuclear RNA (snRNA), small nucleolar RNA (snoRNA) and Y RNA fragments.

Extracellular vesicles are secreted into the extracellular space and can transfer their constituents to other cells by binding and fusing to their plasma membrane (Murphy, de Jong et al. 2019). Constituents of both the extracellular vesicle lipid bi-layer and the extracellular vesicle cargo can be incorporated into the receiving cell. In one embodiment, the transfer of protein from the extracellular vesicle to the receiving cell may determine absorption of extracellular vesicle constituents. In another embodiment, the transfer of lipids from the extracellular vesicle to the receiving cell may determine absorption of extracellular vesicle constituents. In certain embodiments, the human heart cells absorbing the extracellular vesicles may be immune cells, endothelial cells, myocytes and/or fibroblasts. In certain embodiments, the human heart cells absorbing the extracellular vesicles may be in vitro. In certain embodiments, the human heart cells absorbing the extracellular vesicles may be in vivo. Exemplary experimental methods for such determinations are provided in Examples described below. The skilled person will understand that various changes, alternative techniques, or substitutions may be made to the experimental methods provided in these Examples, and that the following Examples are intended to be non-limiting.

The interrogation of extracellular vesicle constituents has uncovered several proteins and nucleic acids related to heart conditions and diseases. By way of example, over-expression of miR-24 improved heart function and attenuated fibrosis in a rodent model of myocardial function (Barile, Lionetti et al. 2014). The person of skill in the art will further recognize that extracellular vesicle constituents may positively impact heart conditions or function (examples including, but not limited to, (Quattrocelli, Crippa et al. 2013), (Yang, Qin et al. 2019), U.S. Pat. No. 9,828,603) or negatively impact heart conditions and heart function (examples including, but not limited to, (Adam, Lohfelm et al. 2012), (Cardin, Guasch et al. 2012), (Cao, Shi et al. 2017), (Zhi, Xu et al. 2019)). Findings as described herein are somewhat surprising, as several extracellular vesicle constituents reported to be substantially absent or devoid herein have clearly been previously shown to have positive effects on heart outcomes (U.S. Pat. No. 9,828,603).

The person of skill in the art having regard to the teachings herein will further recognize that it is contemplated that compositions of extracellular vesicles as described here may be further prepared by performing genetic reprogramming/genetic modification on the human heart cells including, but not limited to, to increase specific constituents (discussed in, for example, (Hall, Prabhakar et al. 2016)), decrease specific constituents (discussed in, for example, (Pfeifer, Werner et al. 2015)), target extracellular vesicles to specific locations (discussed in, for example, (Kooijmans, Schiffelers et al. 2016)), target extracellular vesicles to specific cells (discussed in, for example, (Kooijmans, Schiffelers et al. 2016)), increase the production of extracellular vesicles (discussed in, for example, (Park, Bandeira et al. 2019)), thus boosting extracellular vesicle function, as described in the aforementioned references. By way of example, in certain embodiments it is contemplated that human heart cells as described herein may be subjected to genetic reprogramming to over-express the KCNN4 gene, which may promote extracellular vesicle production. Such approaches may involve lentivirus reprogramming, CRISPR/Cas9 editing, or other methods known to the person of skill such as mini circle DNA, for example.

Inflammation can be a protective response by an immune system to defend against harmful stimuli; however, prolonged inflammation may lead to detrimental conditions. Inflammation can be regulated by immune cells, said immune cells capable of infiltrating tissues, secreting factors and destroying perceived detrimental material. The infiltration of inflammatory immune cells into atrial tissue may be observed in certain subjects with atrial arrhythmias (as discussed in, for example, (Zhou and Dudley 2020)), therefore the presence of inflammatory immune cells in a tissue may indicate inflammation. A person of skill would recognize that immune cells can infiltrate the heart. In certain embodiments, the phrase “human heart cells” may also comprise immune cells. Inflammasomes are receptors, sometimes referred to as sensors, which regulate the inflammatory response and can serve as indicators of potential inflammation with markers of the inflammasome, including, but not limited to caspase-1 activation. The infiltration of inflammatory immune cells into the atrial tissue of a subject with atrial arrhythmia presents the possibility that immune cells may absorb extracellular vesicles. Immune cells may bind and/or secrete molecules including, but not limited to, cytokines, interleukins, interferons, chemokines, complement protein, or any equivalent, to induce an inflammatory response in the surrounding tissue. Increases in immune cell infiltration, secreted immune molecules and inflammasome activation may indicate an increase in inflammation. A person of skill in the art will recognize that many techniques are possible to determine inflammation. The skilled person having regard to the teachings herein will be able to select a suitable method for a given application. Exemplary experimental methods for such determinations are in Examples described below. The skilled person will understand that various changes, alternative techniques, or substitutions may be made to the experimental methods provided in these Examples, and that the following Examples are intended to be non-limiting.

Fibrosis may result in the thickening and/or scarring of the afflicted tissue. Fibrosis may occur due to excess deposition of extracellular matrix components, including, but not limited to, collagen, fibronectrin and fibrin, from fibroblast cells and may result from long-term inflammation (Wynn 2008). It is further thought that fibrosis may disrupt the electrical activity of the heart leading to conditions, including, but not limited to, atrial arrhythmias. A person of skill in the art will recognize that many techniques are possible to determine fibrosis, including but not limited to, for example, hydroxyproline and relative tissue mass. The skilled person having regard to the teachings herein will be able to select a suitable method for a given application. Exemplary experimental methods for such determinations are provided in Examples described below. The skilled person will understand that various changes, alternative techniques, or substitutions may be made to the experimental methods provided in these Examples, and that the following Examples are intended to be non-limiting.

A person of skill in the art will recognize that increases in fibroblast proliferation may promote fibrosis, as taught in, for example, (Kendall and Feghali-Bostwick 2014). Therefore, a person of skill in the art would recognize changes in fibroblast proliferation may be a precursor to fibrosis and atrial arrhythmia, as further taught in, for example, (Ashihara, Haraguchi et al. 2012), and (Aguilar, Qi et al. 2014). A person of skill in the art will recognize that many techniques are possible to quantify fibroblast proliferation. Cellular proliferation is regulated, in part, by transcriptional changes in genes that positively and/or negatively regulate the cell cycle (Liu, Chen et al. 2017). Herein, a positive cell cycle regulatory genes may refer to a gene that, when expressed or activated, may increase cell division by promoting progression into the cell cycle and/or progression through a phase of the cell cycle, including but not limited to, for example Cyclin D, Cyclin E, Cyclin A and Cyclin B. Herein, a negative cell cycle regulatory gene may refer to a gene that, when expressed or activated, may decrease cell division by preventing progression into the cell cycle and/or preventing progression through a phase of the cell cycle, including but not limited to, for example p53, RB, p21, and cyclin-dependent kinase inhibitors (CKI). As taught in, for example, (Bretones, Delgado et al. 2015; Rubin, Sage et al. 2020), regulation of cell cycle genes is thought to be controlled, in part, through transcription factors, transcription factor families and co-factors including, but not limited to, E2F, MYC, p53, RB, P107, P130, and more, which regulate the expression of gene families, including, but not limited to, for example cyclins and cyclin-dependent kinases. Transcriptional regulation of the cell cycle results in complex changes in gene expression of factors influencing entry into the cell cycle from G0 or G1 phases to S-phase, as well as progression through G2 phase and M phase. The skilled person having regard to the teachings herein will be able to select a suitable method for a given application. Exemplary experimental methods for such determinations are provided in Examples described below. The skilled person will understand that various changes, alternative techniques, or substitutions may be made to the experimental methods provided in these Examples, and that the following Examples are intended to be non-limiting.

Derivation of extracellular vesicles may either be undertaken from bodily fluids (examples, including but not limited to, blood, milk, and urine) or conditioned cell culture medium. As bodily fluids may contain extracellular vesicles from many bodily sources, a person of skill will recognize that derivation of extracellular vesicles from cell culture medium minimizes heterogeneity of the vesicle source compared to bodily fluids. In some embodiments, said human heart cells may have been grown in vitro. In certain embodiments, said human heart cells may have been expanded in vitro. As will be understood, the presently described extracellular vesicles compositions, uses and methods thereof, may be amenable to Good Manufacturing Practices (GMP) standards and/or other such pharmaceutical industry standards. In some embodiments, GMP standards may be used, for example, culturing heart explant derived cells as described in U.S. Pat. No. 11,083,756. Standard operating procedures (SOPs) may be developed for producing and using the extracellular vesicle compositions described herein, examples of which are provided in U.S. Pat. No. 11,083,756. In certain embodiments, said human heart cells may have been grown and expanded in vitro using GMP conditions. In certain embodiments, GMP conditions may comprise controlled physiological cell culture conditions, said conditions comprising continuous atmosphere around 1% to around 10% oxygen, and around 1% to around 10% carbon dioxide, relative humidity around 50% to around 90% RH, and temperature around 32° C. to around 42° C., serum-free, xenogen-free growth media, and extracellular vesicle depleted fetal bovine serum. In certain embodiments, said human heart cells may have been expanded in vitro using a GMP compliant enzyme to disassociate cells from culture plates or culture dishes, said GMP compliant enzyme comprising one or more of TrypLE™ Select, collagenase I and collagenase II, or combination thereof. In certain embodiments, said extracellular vesicles are isolated after about 1 hours to about 196 hours or more incubation, any values defining a range therein, including, for example, 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, 31 hrs, 32 hrs, 33 hrs, 34 hrs, 35 hrs, 36 hrs, 37 hrs, 38 hrs, 39 hrs, 40 hrs, 41 hrs, 42 hrs, 43 hrs, 44 hrs, 45 hrs, 46 hrs, 47 hrs, 48 hrs, 49 hrs, 50 hrs, 51 hrs, 52 hrs, 53 hrs, 54 hrs, 55 hrs, 56 hrs, 57 hrs, 58 hrs, 59 hrs, 60 hrs, 61 hrs, 62 hrs, 63 hrs, 64 hrs, 65 hrs, 66 hrs, 67 hrs, 68 hrs, 69 hrs, 70 hrs, 71 hrs, 72 hrs, 73 hrs, 74 hrs, 75 hrs, 76 hrs, 77 hrs, 78 hrs, 79 hrs, 80 hrs, 81 hrs, 82 hrs, 83 hrs, 84 hrs, 85 hrs, 86 hrs, 87 hrs, 88 hrs, 89 hrs, 90 hrs, 91 hrs, 92 hrs, 93 hrs, 94 hrs, 95 hrs, 96 hrs, 97 hrs, 98 hrs, 99 hrs, 100 hrs, 101 hrs, 102 hrs, 103 hrs, 104 hrs, 105 hrs, 106 hrs, 107 hrs, 108 hrs, 109 hrs, 110 hrs, 111 hrs, 112 hrs, 113 hrs, 114 hrs, 115 hrs, 116 hrs, 117 hrs, 118 hrs, 119 hrs, 120 hrs, 121 hrs, 122 hrs, 123 hrs, 124 hrs, 125 hrs, 126 hrs, 127 hrs, 128 hrs, 129 hrs, 130 hrs, 131 hrs, 132 hrs, 133 hrs, 134 hrs, 135 hrs, 136 hrs, 137 hrs, 138 hrs, 139 hrs, 140 hrs, 141 hrs, 142 hrs, 143 hrs, 144 hrs, 145 hrs, 146 hrs, 147 hrs, 148 hrs, 149 hrs, 150 hrs, 151 hrs, 152 hrs, 153 hrs, 154 hrs, 155 hrs, 156 hrs, 157 hrs, 158 hrs, 159 hrs, 160 hrs, 161 hrs, 162 hrs, 163 hrs, 164 hrs, 165 hrs, 166 hrs, 167 hrs, 168 hrs, 169 hrs, 170 hrs, 171 hrs, 172 hrs, 173 hrs, 174 hrs, 175 hrs, 176 hrs, 177 hrs, 178 hrs, 179 hrs, 180 hrs, 181 hrs, 182 hrs, 183 hrs, 184 hrs, 185 hrs, 186 hrs, 187 hrs, 188 hrs, 189 hrs, 190 hrs, 191 hrs, 192 hrs, 193 hrs, 194 hrs, 195 hrs, 196 hrs or more. The skilled person will understand that various changes, alternative techniques, or substitutions may be made to the experimental methods provided in these Examples, and that the following Examples are intended to be non-limiting.

Allorecognition is the ability of an individual organism to distinguish its own tissues from those of other organisms, wherein surface antigens may determine recognition of cells of non-self origin. Extracellular vesicles have different constituents and a person of skill in the art would recognize that each extracellular vesicle composition may or may not activate an immune response after allogenic administration, which may be due to the varied presence of surface antigens. Findings as described herein are somewhat surprising, as the extracellular vesicle composition derived from human heart cells may not induce an immune response. The lack of significant immune response may provide utility beyond other biological agents, which as taught in, for example, (Boehncke and Brembilla 2018) may be hindered by their immunogenicity. Furthermore, certain compositions of extracellular vesicles teach towards a pro-immunogenic nature, such as (Escudier, Dorval et al. 2005). In certain embodiments, substantially immunologically inert is determined by tolerance or lack of reaction following xenogeneic transplantation of any composition of extracellular vesicles above. In certain embodiments, the composition of extracellular vesicles is derived from a xenogeneic source. In certain embodiments, it is contemplated that the extracellular vesicles are derived from a xenogeneic cell line. In certain embodiments, substantially immunologically inert is determined by mixed lymphocyte reaction. Exemplary experimental methods for such determinations are provided in Examples described below. The skilled person will understand that various changes, alternative techniques, or substitutions may be made to the experimental methods provided in these Examples, and that the following Examples are intended to be non-limiting.

Arrhythmia can be detected by analysis of an electrocardiogram, which is a test monitoring the electrical activity of the heart, which provides a graph of voltage versus time of the electrical activity of the heart that detects the small electrical changes in the heart that are a consequence of cardiac muscle depolarization followed by repolarization during each cardiac cycle (heartbeat)). Electrocardiograms can be performed at a medical clinic or can be sampled continuously (i.e., smart watch or inpatient telemetry). A person of skill will recognize that electrocardiogram may be performed as an invasive or non-invasive procedure. The skilled person having regard to the teachings herein will be able to select a suitable method for a given application. Exemplary experimental methods for such determinations are provided in Examples described below. The skilled person will understand that various changes, alternative techniques, or substitutions may be made to the experimental methods provided in these Examples, and that the following Examples are intended to be non-limiting. Additional complementary tests including, but not limited to, electrocardiogram, blood pressure machine, Holter monitor, event monitor, blood tests, echocardiogram, stress test, inpatient telemetry, chest x-ray, smart watch, smart ring, any wearable technology capable of determining heart rate, any equivalent techniques or any combination thereof may be used for diagnosis and/or differential diagnosis of atrial arrhythmia.

Atrial arrhythmias, as taught herein, are a unique class of arrhythmia, which is distinct from other arrhythmias in many ways, such as those arising from the ventricle, including, but not limited to, etiology, pathology, symptoms, treatment options, patient outcomes and patient susceptibility, as discussed in, for example, (Ludhwani, Goyal et al. 2021) and (Nesheiwat, Goyal et al. 2021). Drugs that slow electrical conduction to prevent atrial fibrillation (including but not limited to, for example, flecainide) are contra-indicated in patients with ventricular arrhythmias because they are pro-arrhythmic and increase the risk of death (Ledan 2020). Furthermore, based on the teachings of (Rizvi, DeFranco et al. 2016), a person of skill would recognize chamber-specific differences exist in the mammalian heart, as atrial fibroblasts are different from ventricular fibroblasts.

Atrial arrhythmias may include, but are not limited to, atrial fibrillation, post-operative atrial fibrillation, post-infarction atrial fibrillation, thyrotoxicosis, post-viral atrial fibrillation, alcohol-associated atrial fibrillation, drug-induced atrial fibrillation, viral atrial fibrillation, post-viral atrial fibrillation, COVID-19 atrial fibrillation, post-COVID-19 atrial fibrillation, paroxysmal atrial fibrillation, permanent atrial fibrillation, persistent atrial fibrillation, long-term persistent atrial fibrillation, atrial tachycardia, atrial flutter, familial atrial fibrillation, idiopathic atrial fibrillation, lone atrial fibrillation, and any orphan atrial arrhythmia. Atrial arrhythmias may produce symptoms including, but not limited to, general fatigue, rapid heartbeat, irregular heartbeat, dizziness, shortness of breath, anxiety, syncope, heart failure, neck pounding, weakness, confusion, faintness, sweating, chest pain, chest pressure, chest fluttering, or any combination thereof. In certain embodiments of any of the above compositions or methods, said composition of extracellular vesicles improve one or more symptoms of atrial arrhythmias. In certain embodiments, said composition of extracellular vesicles may reduce the duration of atrial fibrillation, wherein duration may refer to the length of time of a single episode of atrial fibrillation, comprising a reduction of is or more, any values defining a range therein, for example, 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 sec, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years or more. In certain embodiments, said composition of extracellular vesicles may reduce the incidence of atrial fibrillation, wherein incidence may refer to the length of time of a between episodes of atrial fibrillation, comprising a reduction of is or more, any values defining a range therein for example, 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 sec, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years or more. Exemplary experimental methods for such determinations are provided in Examples described below. The skilled person will understand that various changes, alternative techniques, or substitutions may be made to the experimental methods provided in these Examples, and that the following Examples are intended to be non-limiting.

Forms of administration may include, but are not limited to, injections, catheters, solutions, creams, gels, implants, pumps, ointments, emulsions, suspensions, microspheres, particles, microparticles, nanoparticles, liposomes, pastes, patches, tablets, capsules, transdermal delivery devices, sprays, aerosols, or other means familiar to one of ordinary skill in the art. Exemplary experimental methods for such determinations are provided in Examples described below. The skilled person will understand that various changes, alternative techniques, or substitutions may be made to the experimental methods provided in these Examples, and that the following Examples are intended to be non-limiting. The person of skill in the art will be able to select a suitable administration method to suit particular applications and/or particular subject needs.

In another embodiment of any of the compositions or methods above, the extracellular vesicles may be administered by single or multiple injections, wherein the number of injections may be determined as a ratio of the total surface area of the tissue being injected. Without wishing to be bound by theory, a non-limiting example, including injection about every 0.01 cm² to about every 10.00 cm², any values defining a range therein, for example, 0.01 cm² to 0.05 cm², 0.05 cm² to 0.10 cm², 0.15 cm² to 0.20 cm², 0.25 cm² to 0.30 cm², 0.30 cm² to 0.35 cm², 0.35 cm² to 0.40 cm², 0.40 cm² to 0.45 cm², 0.50 cm² to 0.55 cm², 0.55 cm² to 0.60 cm², 0.60 cm² to 0.65 cm², 0.65 cm² to 0.70 cm², 0.70 cm² to 0.75 cm², 0.75 cm² to 0.80 cm², 0.85 cm² to 0.90 cm², 0.90 cm² to 0.95 cm², 0.95 cm² to 1.00 cm², 1.00 cm² to 1.10 cm², 1.10 cm² to 1.20 cm², 1.20 cm² to 1.30 cm², 1.30 cm² to 1.40 cm², 1.40 cm² to 1.50 cm², 1.50 cm² to 1.60 cm², 1.60 cm² to 1.70 cm², 1.70 cm² to 1.80 cm², 1.80 cm² to 1.90 cm², 1.90 cm² to 2.00 cm², 2.00 cm² to 2.10 cm², 2.10 cm² to 2.20 cm², 2.20 cm² to 2.30 cm², 2.30 cm² to 2.40 cm², 2.40 cm² to 2.50 cm², 2.50 cm² to 2.60 cm², 2.60 cm² to 2.70 cm², 2.70 cm² to 2.80 cm², 2.80 cm² to 2.90 cm², 2.90 cm² to 3.00 cm², 3.00 cm² to 3.10 cm², 3.10 cm² to 3.20 cm², 3.20 cm² to 3.30 cm², 3.30 cm² to 3.40 cm², 3.40 cm² to 3.50 cm², 3.50 cm² to 3.60 cm², 3.60 cm² to 3.70 cm², 3.70 cm² to 3.80 cm², 3.80 cm² to 3.90 cm², 3.90 cm² 15 to 4.00 cm², 4.00 cm² to 4.10 cm², 4.10 cm² to 4.20 cm², 4.20 cm² to 4.30 cm², 4.30 cm² to 4.40 cm², 4.40 cm² to 4.50 cm², 4.50 cm² to 4.60 cm², 4.60 cm² to 4.70 cm², 4.70 cm² to 4.80 cm², 4.80 cm² to 4.90 cm², 4.90 cm² to 5.00 cm², 5.00 cm² to 5.10 cm², 5.10 cm² to 5.20 cm², 5.20 cm² to 5.30 cm², 5.30 cm² to 5.40 cm², 5.40 cm² to 5.50 cm², 5.50 cm² to 5.60 cm², 5.60 cm² to 5.70 cm², 5.70 cm² to 5.80 cm², 5.80 cm² to 5.90 cm², 5.90 cm² to 6.00 cm², 6.00 cm² to 6.10 cm², 6.10 cm² to 6.20 cm², 6.20 cm² to 6.30 cm², 6.30 cm² to 6.40 cm², 6.40 cm² to 6.50 cm², 6.50 cm² to 6.60 cm², 6.60 cm² to 6.70 cm², 6.70 cm² to 6.80 cm², 6.80 cm² to 6.90 cm², 6.90 cm² to 7.00 cm², 7.00 cm² to 7.10 cm², 7.10 cm² to 7.20 cm², 7.20 cm² to 7.30 cm², 7.30 cm² to 7.40 cm², 7.40 cm² to 7.50 cm², 7.50 cm² to 7.60 cm², 7.60 cm² to 7.70 cm², 7.70 cm² to 7.80 cm², 7.80 cm² to 7.90 cm², 7.90 cm² to 8.00 cm², 8.00 cm² to 8.10 cm², 8.10 cm² to 8.20 cm², 8.20 cm² to 8.30 cm², 8.30 cm² to 8.40 cm², 8.40 cm² to 8.50 cm², 8.50 cm² to 8.60 cm cm², 8.60 cm² to 8.70 cm², 8.70 cm² to 8.80 cm², 8.80 cm² to 8.90 cm², 8.90 cm² to 9.00 cm², 9.00 cm cm² to 9.10 cm², 9.10 cm² to 9.20 cm², 9.20 cm² to 9.30 cm², 9.30 cm² to 9.40 cm², 9.40 cm² to 9.50 cm², 9.50 cm² to 9.60 cm², 9.60 cm² to 9.70 cm², 9.70 cm² to 9.80 cm², 9.80 cm² to 9.90 cm cm², and 9.90 cm² to 10.00 cm cm². In another embodiment, injection may comprise 1 to about 500 injections, including, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 injections.

Exemplary experimental methods for such determinations are provided in Examples described below.

The skilled person will understand that various changes, alternative techniques, or substitutions may be made to the experimental methods provided in these Examples, and that the following Examples are intended to be non-limiting.

In another embodiment of any of the compositions or methods above, the extracellular vesicle composition may be administered by dosage form in syringe. In certain embodiments, the dosage of the extracellular vesicle composition may be related to the number of particles in a unit volume, such as number of particles per milliliter. In certain embodiments, the dosage of the extracellular vesicle composition may be from about 10² particles/mL to about 10²⁰ particles/mL, any values defining a range therein, including, for example, 1×10² particles/mL to 5×10² particles/mL, 5×10² particles/mL to 1×10³ particles/mL, 1×10³ particles/mL to 5×10³ particles/mL, 5×10³ particles/mL to 1×10⁴ particles/mL, 1×10⁴ particles/mL to 5×10⁴ particles/mL, 5×10⁴ particles/mL to 1×10⁵ particles/mL, 1×10⁵ particles/mL to 5×10⁵ particles/mL, 5×10⁵ particles/mL to 1×10⁶ particles/mL, 1×10⁶ particles/mL to 5×10⁶ particles/mL, 5×10⁶ particles/mL to 1×10⁷ particles/mL, 1×10⁷ particles/mL to 5×10⁷ particles/mL, 5×10⁷ particles/mL to 1×10⁸ particles/mL, 1×10⁸ particles/mL to 5×10⁸ particles/mL, 5×10⁸ particles/mL to 1×10⁹ particles/mL, 1×10⁹ particles/mL to 5×10⁹ particles/mL, 5×10⁹ particles/mL to 1×10¹⁰ particles/mL, 1×10¹⁰ particles/mL to 5×10¹⁰ particles/mL, 5×10¹⁰ particles/mL to 1×10¹¹ particles/mL, 1×10¹¹ particles/mL to 5×10¹¹ particles/mL, 5×10¹¹ particles/mL to 1×10¹² particles/mL, 1×10¹² particles/mL to 5×10¹² particles/mL, 5×10¹² particles/mL to 1×10¹³ particles/mL, 1×10¹³ particles/mL to 5×10¹³ particles/mL, 5×10¹³ particles/mL to 1×10¹⁴ particles/mL, 1×10¹⁴ particles/mL to 5×10¹⁴ particles/mL, 5×10¹⁴ particles/mL to 1×10¹⁵ particles/mL, 1×10¹⁵ particles/mL to 5×10¹⁵ particles/mL, 5×10¹⁵ particles/mL to 1×10¹⁶ particles/mL, 1×10¹⁶ particles/mL to 5×10¹⁶ particles/mL, 5×10¹⁶ particles/mL to 1×10¹⁷ particles/mL, 1×10¹⁷ particles/mL to 5×10¹⁷ particles/mL, 5×10¹⁷ particles/mL to 1×10¹⁸ particles/mL, 1×10¹⁸ particles/mL to 5×10¹⁸ particles/mL, 5×10¹⁸ particles/mL to 1×10¹⁹ particles/mL, 1×10¹⁹ particles/mL to 5×10¹⁹ particles/mL and 5×10¹⁹ particles/mL to 1×10²⁰ particles/mL.

In another embodiment of any of the compositions or methods above, the dosage of the extracellular vesicle composition may be from about 10² particles to about 10²⁰ particles, any values defining a range therein, including, for example, 1×10² particles to 5×10² particles, 5×10² particles to 1×10³ particles, 1×10³ particles to 5×10³ particles, 5×10³ particles to 1×10⁴ particles, 1×10⁴ particles to 5×10⁴ particles, 5×10⁴ particles to 1×10⁵ particles, 1×10⁵ particles to 5×10⁵ particles, 5×10⁵ particles to 1×10⁶ particles, 1×10⁶ particles to 5×10⁶ particles, 5×10⁶ particles to 1×10⁷ particles, 1×10⁷ particles to 5×10⁷ particles, 5×10⁷ particles to 1×10⁸ particles, 1×10⁸ particles to 5×10⁸ particles, 5×10⁸ particles to 1×10⁹ particles, 1×10⁹ particles to 5×10⁹ particles, 5×10⁹ particles to 1×10¹⁰ particles, 1×10¹⁰ particles to 5×10¹⁰ particles, 5×10¹⁰ particles to 1×10¹¹ particles, 1×10¹¹ particles to 5×10¹¹ particles, 5×10¹¹ particles to 1×10¹² particles, 1×10¹² particles to 5×10¹² particles, 5×10¹² particles to 1×10¹³ particles, 1×10¹³ particles to 5×10¹³ particles, 5×10¹³ particles to 1×10¹⁴ particles, 1×10¹⁴ particles to 5×10¹⁴ particles, 5×10¹⁴ particles to 1×10¹⁵ particles, 1×10¹⁵ particles to 5×10¹⁵ particles, 5×10¹⁵ particles to 1×10¹⁶ particles, 1×10¹⁶ particles to 5×10¹⁶ particles, 5×10¹⁶ particles to 1×10¹⁷ particles, 1×10¹⁷ particles to 5×10¹⁷ particles, 5×10¹⁷ particles to 1×10¹⁸ particles, 1×10¹⁸ particles to 5×10¹⁸ particles, 5×10¹⁸ particles to 1×10¹⁹ particles, 1×10¹⁹ particles to 5×10¹⁹ particles and 5×10¹⁹ particles to 1×10²⁰ particles. In certain embodiments, the dosage of the extracellular vesicle composition may be related to the total number of particles received. In other embodiments, the dosage of the extracellular vesicle composition may be related to the number of particles received in each individual injection. In certain other embodiments, the dosage of the extracellular vesicle composition may be related to the sum of extracellular vesicles received over multiple injections, said injections may be received at the same time or different times. Pharmaceutical formulations of the present invention can be prepared by procedures known in the art using well-known and readily available ingredients. For example, the compounds can be formulated with common excipients, diluents, or carriers, and formed into tablets, capsules, suspensions, powders, and the like. Examples of excipients, diluents, and carriers that are suitable for such formulations include the following: fillers and extenders (e.g; starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; coloring agents; perfuming agents; flavoring agents; lubricants (e.g., talc, calcium and magnesium stearate); solid polyethyl glycols; and mixtures thereof.

The person of skill in the art will understand that biomolecules and/or compounds described herein may be provided in pharmaceutical compositions together with a pharmaceutically acceptable diluent, carrier, or excipient, and/or together with one or more separate active agents or drugs as part of a pharmaceutical combination or pharmaceutical composition. In certain embodiments, the biomolecules, compounds, and/or pharmaceutical compositions may be administered in a treatment regimen simultaneously, sequentially, or in combination with other drugs or pharmaceutical compositions, either separately or as a combined formulation or combination. In certain embodiments, the extracellular vesicle composition may be administered in a treatment regimen simultaneously, sequentially, or in combination with other drugs, pharmaceutical compositions or treatments, either separately or as a combined formulation or combination, to treat or prevent atrial arrhythmia. In certain embodiments, the extracellular vesicle composition may be administered in a treatment regimen simultaneously, sequentially, or in combination with rhythm control drugs (including but not limited to, for example, flecainide, propafenone, quinidine, sotalol, amiodarone and dronedarone), rate control drugs (including but not limited to, for example, beta blockers, calcium channel blocks and cardiac glycosides) or surgical treatment (including but not limited to, for example, electrical cardioversion, catheter ablation, pacemaker insertion, defibrillator implantation, and the Maze procedure), either separately or as a combined formulation or combination, to treat or prevent atrial arrhythmia.

The compositions can be constituted such that they release the active ingredient only or preferably in a particular location or cell type, and/or possibly over a period of time (i.e., a sustained-release formulation). Such combinations provide yet a further mechanism for controlling release kinetics and distribution. The coatings, envelopes, and protective matrices may be made, for example, from polymeric substances or waxes and the pharmaceutically acceptable carrier. In certain further embodiments of any of the above compositions or methods, the extracellular vesicles are modified to limit their absorption solely to fibroblasts, cardiomyocytes, endothelial cells, and/or immune cells, or any desired cell or tissue type.

As used herein, the expression “differentially expressed” may refer to a change in expression or level for a given factor, such as, but not limited to, a protein, an RNA, an mRNA, a miRNA, biomolecule or any bioactive cellular component from one sample in comparison to another sample. Samples examined for differential expression may be different cell types, different experimental conditions, different genetic manipulations or any other change in parameter that may be known to the person of skill. In certain embodiments, the expression “differentially expressed” may refer to any increase or decrease in expression in a biomolecule or a bioactive cellular component, such as, but not limited to, a protein, an RNA, a miRNA, an mRNA, or any equivalent bioactive component known to a person of skill in the art.

In certain embodiments, the expression “differentially expressed” may refer to a significant increase or decrease in expression in a cellular component, such as but not limited to, a protein, an RNA, a miRNA, an mRNA, or any equivalent bioactive component known to a person of skill in the art. In certain embodiments, the expression “differentially expressed” may refer to a log 2 fold ≥ or ≤1.5 and p-value <0.05 increase or decrease in expression in a bioactive cellular component, such as but not limited to, a protein, an RNA, a miRNA, an mRNA, or any component known to a person of skill in the art.

In certain embodiments, a differentially expressed miRNA may also comprise mRNA target(s) of the differentially expressed miRNA. As would be known to a person of skill, miRNA function by regulating post-transcriptional gene expression, generally through binding to complementary sequences on mRNA transcripts. As such, it is contemplated that in certain embodiments described herein, “differentially expressed miRNA” may include mRNA that may have a putative or predicted miRNA binding site or consensus site for the miRNA(s) that is differentially expressed, and may also encompass protein encoded by mRNA which may contain a putative or predicted miRNA binding site of a differentially expressed miRNA. A person of skill in the art, in light of the teachings herein, would be able to select an appropriate technique or algorithm, such as but not limited to miRWalk (Sticht, De La Torre, et al. 2018), that may provide a list of mRNA target sequences for a given miRNA. In certain embodiments, an algorithm or technique to determine mRNA targets of differentially expressed miRNA may comprise analysis of the 3′UTR and 5′ UTR of mRNAs for miRNA consensus sites or binding sites. In certain embodiments, an algorithm or technique to determine mRNA targets of differentially expressed miRNA may comprise analysis of the 3′UTR of mRNA for miRNA consensus sites or binding sites. In certain embodiments, differentially expressed miRNA may be provided to miRWalk to determine differentially expressed mRNA(s).

In certain embodiments, without wishing to be bound by theory, it is contemplated that one or more differentially expressed components comprising a heart derived extracellular vesicle, may mediate their activity. In certain embodiments, without wishing to be bound by theory, it is contemplated that a plurality of differentially expressed components comprising a heart derived extracellular vesicle, may mediate their activity.

As used herein, the term “unique” may comprise one or more factors that are present in a condition that are not present in another condition, or present in minimal quantities in another condition. In certain embodiments, the term “unique” may refer to a group of factors, such as biomolecules, bioactive components, protein, mRNA, miRNA present in, for example, an extracellular vesicle or extracellular vesicle cargo. It is contemplated that, for example, a unique miRNA may comprise a plurality of miRNA's in combination that is unique or a unique protein may comprise a plurality proteins in combination that is unique. In certain embodiments, the term “unique” may refer to the absence of specific factors, such as bioactive components, proteins, mRNA, miRNA present in, for example, an extracellular vesicle or extracellular vesicle cargo. In certain embodiments, the composition of extracellular vesicles may comprise one or more unique protein(s). In certain embodiments, the composition of extracellular vesicles may comprise one or more unique protein(s) as compared with extracellular vesicles isolated from sources not comprising human heart tissue.

A person of skill, in light of the teachings herein, would appreciate that atrial arrhythmias may be induced, triggered, and/or caused by many factors. A person of skill would appreciate that models, such as but not limited to, sterile pericarditis, ventricular dysfunction, infarction, and coronary artery ligation may induce atrial arrhythmias or increase the likelihood of developing atrial arrhythmias.

In certain embodiments, it is contemplated that human heart derived extracellular vesicles may be applied or delivered to a subject in a gel. In certain embodiments, the gel may be a biomaterial gel. As used herein, a “biomaterial gel” may refer to a gel that is substantially comprised of one or more biological material. In certain embodiments, the gel may be an agarose gel. In certain embodiments, the biomaterial gel may be impregnated with human heart derived extracellular vesicles. Impregnated, as used herein, may refer to the insertion or application of extracellular vesicles into another object or material, such as a gel. Impregnating the material may result in substantially homogenous distribution of extracellular vesicles throughout the material, such that the concentration of vesicles is about equal throughout the material. A person of skill in the art, in light of the teachings herein, would be able to select appropriate conditions, such as but not limited to, agarose type or agarose concentration, to achieve the human heart derived extracellular vesicle impregnated gel.

As used herein, the term “payload” may refer to the components comprising an extracellular vesicle, such as, its cargo. As used herein, the terms “constituents”, “cargo”, and “payload” may be used interchangeably to refer to the factors comprising an extracellular vesicle. As would be known to a person of skill, EV cargo, such as for example proteins and RNA, may be dependent upon tissue cell source, and methodological factors, such as, but not limited to, different media, EV collection conditions, protein quantification techniques, RNA quantification techniques, techniques for the analysis of protein quantification, and techniques for the analysis of RNA quantification. A person of skill would appreciate that comparisons made between samples, such as those containing extracellular vesicles, may be more consistent when fewer variables are changed. As such, a comparison between the EV cargo from different tissues should also use substantially similar techniques to derive, quantify and analyze the EVs to ensure the most accurate results for determination of variable-specific expression level changes or differential expression analysis.

The following examples are provided for illustrative purposes and are intended for the person of skill in the art. These examples are provided to demonstrate certain embodiments as described herein, and should not be seen as limiting in any way.

### EXAMPLES

### Example 1

### Material and Methods

### Heart Explant Derived Cell (EDC) Culture and Extracellular Vesicle (EV) Isolation

Human heart explant derived cells (EDCs) were obtained from left atrial appendages donated by patients undergoing clinically indicated heart surgery after informed consent under a protocol approved by the University of Ottawa Heart Institute Research Ethics Board. EDCs were cultured as previously described (Davis, Kizana et al. 2010; Latham, Ye et al. 2013; Mount, Kanda et al. 2019). Briefly, cardiac biopsies were minced, digested with appropriate enzyme, for example, but not limited to GMP-grade blend of collagenase I/II (Roche) and plated within Nutristem® media (Biological Industries) exposed to physiologic (5%) oxygen in a GMP cell manufacturing facility (Mount, Kanda et al. 2019). Once a week for about 4 weeks, EDCs were collected from the plated tissue using TrypLE® Select (Thermo Fischer Scientific) for direct experimentation.

Conditioned media was collected after 48 hours of culture in 1% EV-depleted serum (System Biosciences) and 1% oxygen for centrifugation at 10,000 g for 30 minutes and 100,000 g for 3 hours to pellet EVs (Kanda, Alarcon et al. 2018; Villanueva, Michie et al. 2019). Extracellular vesicle (EV) content, size and surface marker expression were analyzed using acetylcholinesterase activity (Fluoro-Cet, Systems Biosciences), nanoparticle tracking (Nanosight) and candidate antibody array (Exo-Check®, Systems Biosciences) analysis.

EV miRNA and Proteome Analysis

The miRNA content within EDC EVs was profiled using multiplex fluorescent oligonucleotide-based miRNA detection (Human v3, Nanostring), as previously described (Kanda, Alarcon et al. 2018; Villanueva, Michie et al. 2019). Briefly, RNA was extracted (QIAGEN) and quantified (Agilent 2100 Bioanalyzer, Agilent) prior to profiling (Counter Human V3 miRNA Expression Assay). Image quality was evaluated (nSolver®) and discarded if the percent field of view and sample binding density exceeded prespecified standards. Background subtraction was performed using the mean of negative controls plus two standard deviations. Counts were normalized using trimmed-mean of M values and differentially expressed miRNA were identified using the generalized linear model likelihood-ratio-test.

EDC EVs were lysed (8 M urea, 100 mM HEPES, 5% glycerol, and 0.5% n-dodecyl 3-d-maltoside; Thermo Fischer Scientific), reduced (tris(2-carboxyethyl) phosphine alkylated with iodoacetamide), and digested (1.5 μL of 0.3 μg/L trypsin/Lys-C solution; Promega) prior to formic acid treatment, desalination (C18 TopTips; Glygen) and vacuum drying. Protein samples were analyzed using an Orbitrap Fusion mass spectrometer (Thermo Fisher Scientific) coupled to a UltiMate 3000 nanoRSLC (Dionex, Thermo Fisher Scientific) as previously described (Risha, Minic et al. 2020). Using MaxQuant software, peptides were searched against the human Uniprot FASTA database with a false discovery rate of 1% (Cox and Mann 2008). Pathway analysis terms were extracted from the Reactome database (Sidiropoulos, Viteri et al. 2017) for network analysis (Cytoscape) (Merico, Isserlin et al. 2010). Only proteins found in at least 2 biological replicates were considered for analysis.

Flow cytometry was performed on EVs that were individually labelled for CD9 (312106, BioLegend), CD63 (353004, BioLegend), or CD81 (349506, BioLegend) using a CytoFLEX S Beckman Coulter flow cytometer (Welsh, Jones et al. 2020). Light scatter was calibrated using National Institute of Standards and Technology Traceable Size Standards (Thermo Fischer Scientific) while fluorescence was calibrated using Molecules of Equivalent Soluble Fluorochrome beads (BD Biosciences) for analysis using FCMPASS (v3.07, National Cancer Institute) and FlowJo (V10.7, BD Biosciences) (Welsh and Jones 2020).

### Atrial Fibroblast Isolation

Primary cultures of rat atrial fibroblasts were isolated from the hearts of 8-9-week-old Sprague-Dawley rats using an enzymatic digestion (Collagenase Type II, Worthington Biochemical) at 37° C. Cells were cultured in 5% CO₂ in Dulbecco's Modified Eagle high glucose Medium (DMEM), supplemented with 10% fetal bovine serum (FBS), 1% 1-glutamine and 1% penicillin-streptomycin (Thermo Fischer Scientific). Second or third generation fibroblasts were used in all subsequent experiments.

### EV Uptake In Vitro

The uptake of EVs in cardiac cells was evaluated in a series of cell lines using flow cytometry. Rat atrial fibroblasts, neonatal rat ventricular cardiomyocytes and human umbilical vein endothelial cells (HUVECs; Lonza) were cultured in 108 EVs labelled with 1,1′-Dioctadecyl-3,3,3′,3′-Tetramethylindocarbocyanine Perchlorate (Dil). Neonatal rat ventricular myocytes (NRVMs) were prepared as described from 2 day old Sprague-Dawley rats (Harlan) (Kapoor, Liang et al. 2013; Liang, Yuan et al. 2015). HUVECs were cultured according to the manufacturer's directions.

### In Vivo Efficacy Study

Female and male Sprague-Dawley rats (6 months old, Charles River) underwent induction of sterile pericarditis or sham operation under a protocol approved by the University of Ottawa Animal Care Committee. The detailed protocol was registered a priori within the Open Science Framework. A one-way study was designed to test if intramyocardial injection of EDC EVs at the time of sterile pericarditis induction significantly reduced the incidence of AF by invasive electrophysiological testing (primary outcome). A rat model of sterile pericarditis following talc application was used (Huang, Chen et al. 2016). To increase the rigour and reproducibility of the study for post-operative atrial fibrillation (POAF), we studied 6-month-old female and male rats. We assumed the incidence of AF would be 0.6 after talc treatment and that EV treatment would reduce AF incidence to 0.2. Sex was assumed not to alter the incidence of inducible AF. Based on these assumptions, group sample sizes of 34 rats (17 female+17 male) would achieve an 83% power to detect superiority using a two-sided Mann-Whitney test (probability of a false positive result (alpha error)=0.05).

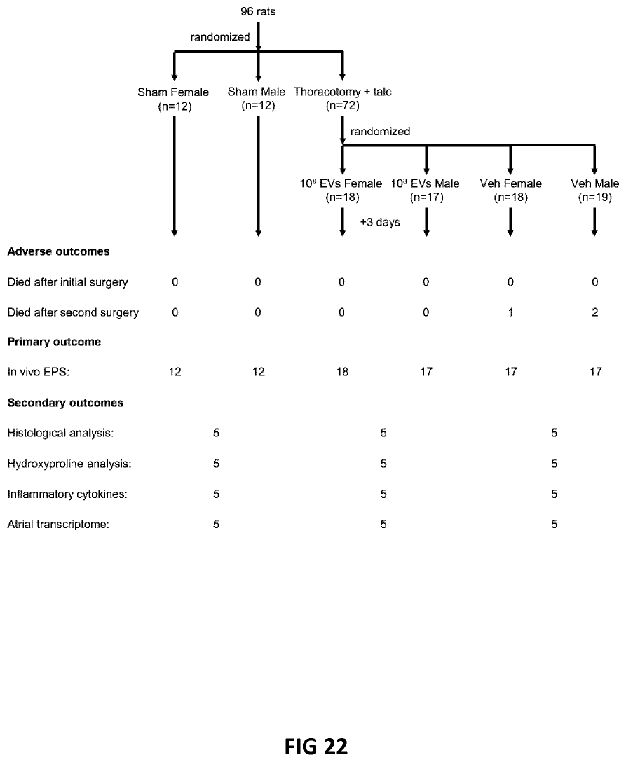

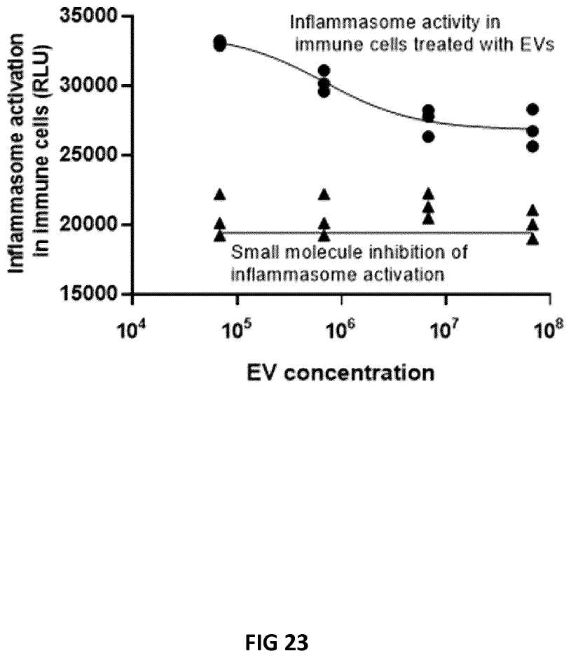

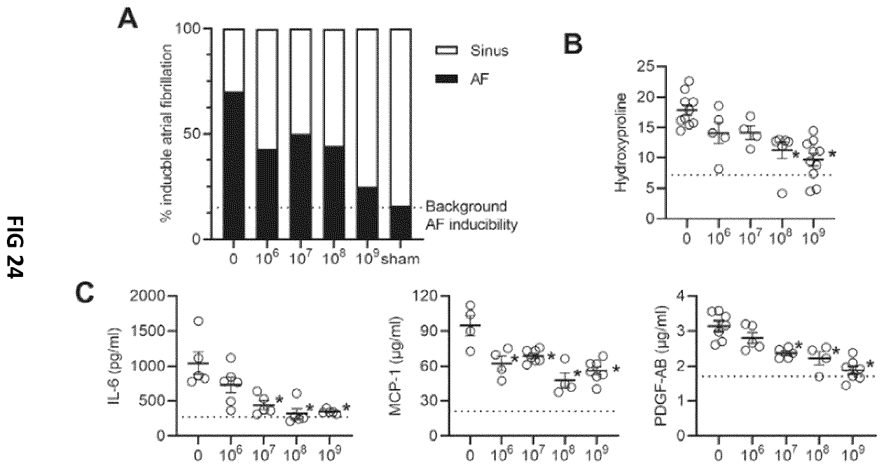

Rats were fed rat chow and housed under a 12:12-hour light/dark cycle at 21° C. and 50% humidity. All animals had free access to tap water and food. After preoperative buprenorphine (0.03 mg/kg subcutaneous), rats were anesthetized with 3% isoflurane, intubated, and ventilated. The thorax was shaved and sterilized with 2% w/v chlorhexidine gluconate in 70% v/v isopropyl alcohol. Animals were then randomized to sham operation (n=24; 12 female, 12 male), induction of sterile pericarditis with intramyocardial injection of 10⁸ atrial EVs (n=35; 18 female, 17 male), or induction of sterile pericarditis with intramyocardial injection of vehicle (n=34; 17 female, 17 male) using a sealed envelope approach. Animals randomized to sterile pericarditis underwent a thoracotomy and the atrial surfaces were generously dusted with sterile talcum powder (Thermo Fischer Scientific). Animals randomized to sham underwent a superficial incision that was closed in a manner indistinguishable from thoracotomy animals. Intramyocardial injections were performed using a total volume of 100-μL injected using a Hamilton microsyringe (31-gauge needle) into the left atrial wall at 5 separate injection points (Cardin, Guasch et al. 2012). After surgery, animals were placed in a 30° C. incubator with supplemental oxygen and moistened food until they returned to a physiological state. Additional doses of buprenorphine (0.03 mg/kg subcutaneous) were administered 6 and 12 hours postoperatively. A University of Ottawa Animal Care Technician monitored animals twice daily for 2 days after surgery. Lab staff was blinded to the treatment received and analysis was conducted by individuals blinded to group allocation. Group allocations were kept in a separate password protected list for unblinding after analysis of primary study outcome was completed. Ninety-six rats underwent surgery, and all completed the study with no adverse events or protocol deviations (FIG. 22).

Three days after surgery, all rats underwent invasive electrophysiological testing (Cardin, Guasch et al. 2012). After intraperitoneal injection of sodium pentobarbital (40 mg/kg), a 1.6F octopolar catheter with 1 mm interelectrode spacing (Millar) was inserted into the right atrium via the jugular vein for stimulation and recording. The surface electrocardiogram and intracardiac electrograms was continuously monitored (AD Instruments). Atrioventricular nodal refractory period was determined as the longest S1-S2 interval that failed to conduct to the ventricle using twice-threshold, 2-ms, square-wave pulses after a 10-stimulus drive train (S1, 100-ms cycle length) followed by an S2 decremented in 2-ms intervals. If that failed to induce AF, 10-30 seconds of atrial burst pacing was performed at cycle lengths that ranged between 20 and 80 ms. AF was defined as rapid and fragmented atrial electrograms, the absence of discernible P waves on the surface electrocardiogram and an irregular ventricular rhythm that lasted for at least 500 ms (Kapoor, Liang et al. 2013). The total time of the AF episode was defined as the sum of the AF duration from the longest AF episode recorded. At the end of the study, rats were sacrificed by exsanguination under pentobarbital anesthesia after displaying absence of withdrawal reflex to toe pinch.

Histolomical analysis and quantification of fibrosis Atria were collected, fixed and sectioned for histological analysis of inflammatory infiltrates (hematoxylin and eosin (H&E), activated T lymphocytes (CD3 (ab16669, abcam) and CD4 (ab237722, abcam), macrophage infiltration/polarization (CD68 (ab125212, abcam) and CD163 (ab182422, abcam) and neutrophils (CD11b (ab133357, abcam)). Hematoxylin and eosin staining was quantified using freely available ImageJ software with the color deconvolution plugin, which separates the hematoxylin component and the eosin component, allowing for each stain to be quantified separately. The total tissue area was determined on the whole section while the area of infiltrates was determined using ImageJ's threshold, obtained after H&E colour deconvolution. This allowed calculation of the percentage of infiltrates (infiltrates divided by whole area) (Gray, Wright et al. 2015; El Harane, Kervadec et al. 2018).

Left atria collagen content was quantified using an unbiased measure of hydroxyproline measurement which reflects the overall degree of myocardial fibrosis (Jamall, Finelli et al. 1981; He, Gao et al. 2011).

### Atrial Fibroblast Transcriptome Analysis

The influence of EV treatment on atrial gene expression was evaluated using RNA sequencing (RNAseq). After atrial RNA isolation, rRNA was removed (Ribo-Zero rRNA Removal Kit, Illumina) for cDNA library preparation followed by sequencing (45 million reads per sample). Reads were first mapped using Bowtie2 prior to gene expression quantification (RSEM v1.2.15. TMM). Genes with a 1.5-fold change in expression (p<0.05) were considered differentially expressed for Ingenuity pathway and network analysis (Qiagen).

### Inflammatory Cytokines

Atrial tissue was minced and homogenized using a tissue homogenizer (TissueRuptor, Qiagen) according to the manufacturer's protocol. Inflammatory cytokines and chemokines were measured using a multiplex Luminex-based assay (LXSARM, R&D Systems). Each sample was run in duplicate in a 96-well plate. Four analytes (IL13, IL2, IL18, TNFα) were measured using a MAGPIX system (C4447b).

Acquired mean fluorescence data were analyzed and calculated by the xPONENT software. IL-6 (ERA32RB, Invitrogen), PDGF-AB (ab213906, abcam) and MCP-1 (ab100778, abcam) were quantified using commercially available enzyme-linked immunosorbent (ELISA) assays.

### Fibroblast Proliferation

Post-operative atrial fibrillation (POAF) was modeled by exposing normal rat atrial fibroblasts to interleukin-6 (IL-6) or transforming growth factor beta 1 (TGFβ1) to recapitulate the inflammatory in vivo environment (Narikawa, Umemura et al. 2018). Proliferation of atrial fibroblasts at baseline and after treatment with IL6, TGFβ1, and/or EDC EVs was evaluated by staining for the nuclear incorporation of the thymidine analogue 5-ethynyl-2′-deoxyuridine (EdU) (17-10525; Millipore) and DAPI. Manual cell counts were performed to confirm the findings. Population doubling time was measured using WST-8 assay (Dojindo). Cell cycle distribution was evaluated using flow cytometry according to the manufacturer's instructions (4500-0220, Guava). Briefly, fibroblasts were cultured in serum free media for 24 hours prior to 24 hours of culture within 10⁸ atrial EVs, IL-6, TGFβ1, aphidicolin (APC) (Sigma), used as a G1 cell arrest control, or vehicle. Flow cytometry was performed to quantify populations within G0/G1, S, and G2/M phases of cell cycle for each treatment (Guava, Millipore Sigma). To further explore the effect of EDC-EVs on atrial fibroblasts, commercially available enzyme-linked immunosorbent (ELISA) assays were performed to look at the molecular regulators of cell cycle progression. Briefly, atrial fibroblasts were lysed according to the manufacturer's instructions and ELISAs were performed for Cyclin A2 (MBS7211946, MyBioSource), Cyclin B1 (MBS9328611, MyBioSource), Cyclin D (MBS721009, MyBioSource) and Cyclin E (MBS1600300, MyBioSource).

Statistical analysis All statistical tests used and graphical depictions of data (means and error bars, or box and whisker plots) are defined within the figure legends for the respective data panels. All data is presented as mean±standard error of the mean. To determine if differences existed within groups, data was analyzed by a one-way or repeated measures ANOVA (SPSS v20.0.0); if such differences existed, Bonferroni's corrected t-test was used to determine the group(s) with the difference(s). In all cases, variances were assumed to be equal and normality was confirmed prior to further post-hoc testing. Differences in categorical measures were analyzed using a Chi Square test. A final value of P<0.05 was considered significant for all analyses.

### Results

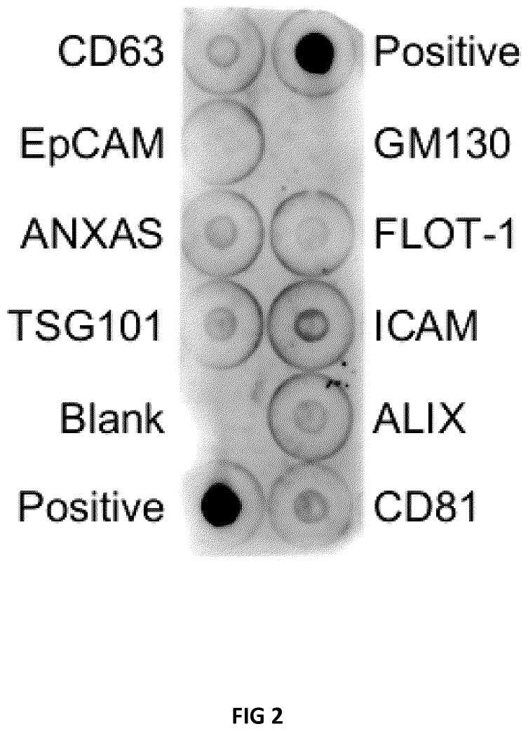

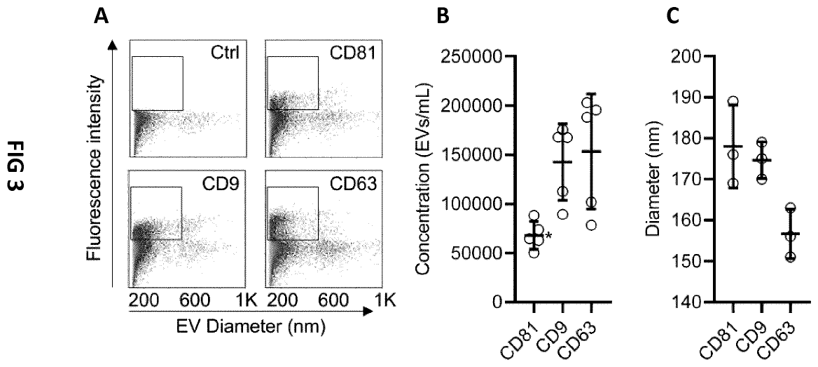

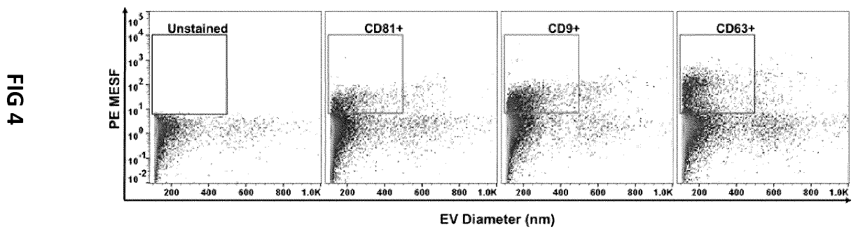

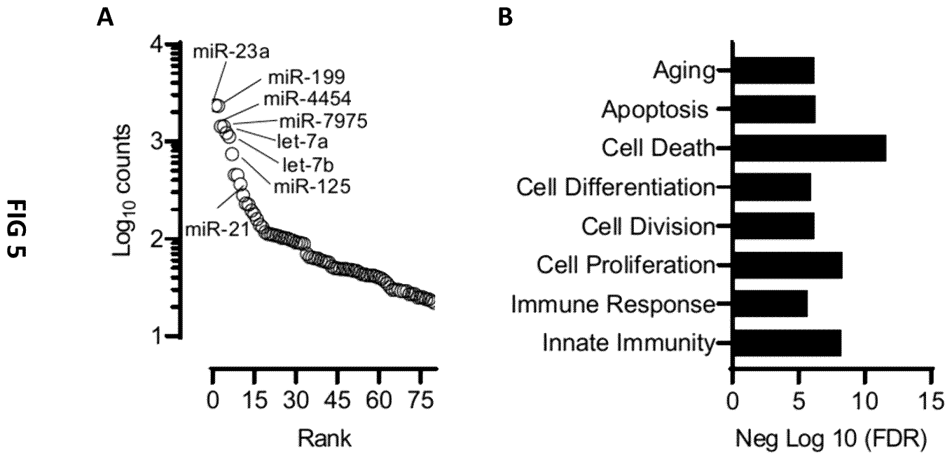

Human atrial EVs contain anti-fibrotic anti-inflammatory transcripts and proteins Human EDCs were cultured in a clinical cell manufacturing facility from atrial appendage biopsies using serum-free xenogen-free culture conditions (Mount, Kanda et al. 2019). EVs were isolated from conditioned media after 48 hours in 1% oxygen, basal media conditions (Kanda, Alarcon et al. 2018; Mount, Kanda et al. 2019). In keeping with accepted definitions, EDC EVs represented a polydisperse population of particles that ranged from 95 to 170 nm (132±7 nm). These microparticles contained transmembrane (CD63, CD81, FLOT1, ICAM1, EpCam) and cytosolic (ALIX, ANXA5 and TSG101) markers indicative of EV identity (Lotvall, Hill et al. 2014) while lacking evidence for cellular contaminants (GM130; FIG. 2). Flow cytometry demonstrated significant enrichment of micro-particles with the prototypical EV markers CD9, CD63, CD81 (FIGS. 3-4). Interestingly, there were fewer particles that expressed CD81 (˜2-fold less, p<0.05 vs. CD9 or CD63) while smaller particles were more apt to express CD63 (p<0.05 vs. CD9 or CD81) (FIGS. 3B-C). Acetylcholinesterase activity was also present and correlated with nanoparticle particle tracking content. Thus, confirming the presence of a functional extracellular membrane-bound protein associated with EVs and providing a rapid means of confirming EV concentration.

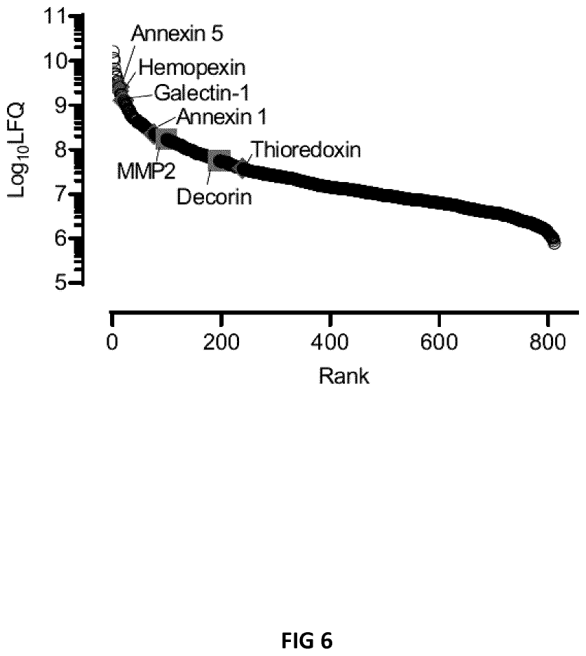

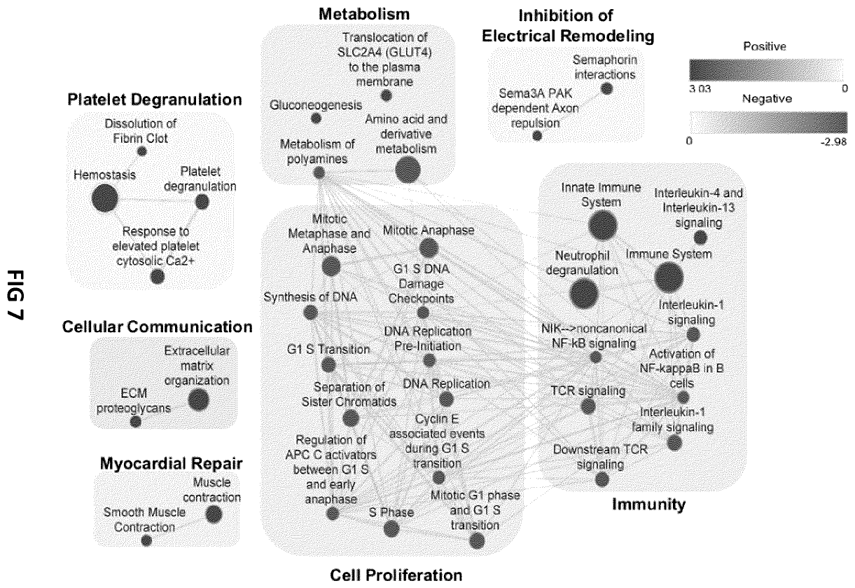

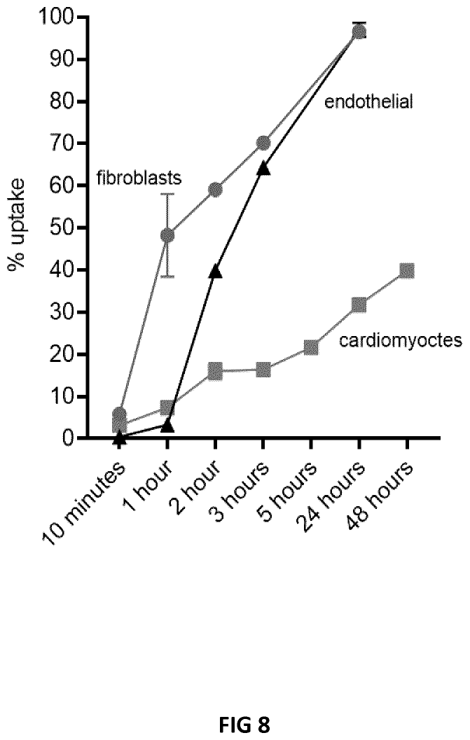

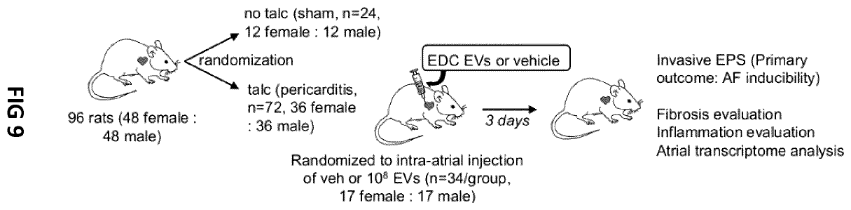

The cargo within EDC EVs was enriched with 83 miRNA transcripts associated with reducing inflammation, stimulating angiogenesis, and suppressing fibrosis (FIG. 5, Table 1). Interestingly, the most abundant miRNAs (1000+counts) were associated with altered cell division/proliferation (let-7a, miR23a and miR-199a) and fibrosis (let-7a and miR-199a). Although EDC EVs contained miR-21, a transcript known to promote fibrosis and AF susceptibility (Cardin, Guasch et al. 2012), they lacked all other known pathological transcripts (miR-1 (Girmatsion, Biliczki et al. 2009)), miR-133 (Tsoporis, Fazio et al. 2018), miR-328 (Small, Frost et al. 2010) and miR-590 (Shan, Zhang et al. 2009)) and included miRNAs associated with reduced fibrotic atrial remodeling (miR-26 (Luo, Pan et al. 2013) and miR-29 (van Rooij, Sutherland et al. 2008)). Within the 811 proteins identified within EDC EVs (FIGS. 6-7), the proteome was enriched with 196 proteins associated with inflammation (Wilcoxon rank sum test p<0.003) which directly influenced both chemotaxis (i.e., annexin A1 and annexin-5) (Lim, Solito et al. 1998; Ewing, de Vries et al. 2011; Burgmaier, Schutters et al. 2014; Vital, Becker et al. 2016; de Jong, Pluijmert et al. 2018), and macrophage function (i.e., galectin-1, hemopexin and thioredoxin) (Correa, Sotomayor et al. 2003; Liang, Lin et al. 2009; El Hadri, Mahmood et al. 2012; Jung, Kwak et al. 2017; Canesi, Mateo et al. 2019). Among the 28 proteins implicated in fibrosis, decorin (a transforming growth factor 1 beta (TGF-β1) inhibitor) and matrix metalloproteinase 2 were highly expressed (Onozuka, Kakinuma et al. 2011; Radbill, Gupta et al. 2011; Baghy, Iozzo et al. 2012; Parichatikanond, Luangmonkong et al. 2020). Enrichment mapping of the EV proteome predicted important roles in cell cycle regulation, inflammation and cellular signaling.

Taken together, this data supports the notion that EDCs produce a defined EV product containing an anti-fibrotic, anti-inflammatory cargo with the potential to alter the fundamental drivers of POAF.

TABLE 1 shows exemplary miRNA identified using multiplex fluorescent oligonucleotide-based miRNA detection within extracellular vesicles derived from heart explant derived cells. hsa, Homo sapiens

| | Average |
|---|---|
| miRNA | Counts |
| hsa-miR-23a-3p | 2355 |
| hsa-miR-199a-3p + | 2326 |
| hsa-miR-199b-3p | |
| hsa-miR-4454 + | 1426 |
| hsa-miR-7975 | |
| hsa-let-7a-5p | 1221 |
| hsa-let-7b-5p | 1122 |
| hsa-miR-125b-5p | 743 |
| hsa-miR-100-5p | 453 |
| hsa-miR-29b-3p | 450 |
| hsa-miR-21-5p | 362 |
| hsa-miR-191-5p | 279 |
| hsa-miR-199b-5p | 230 |
| hsa-miR-29a-3p | 224 |
| hsa-miR-22-3p | 195 |
| hsa-let-7i-5p | 178 |
| hsa-miR-181a-5p | 159 |
| hsa-miR-25-3p | 139 |
| hsa-miR-127-3p | 131 |
| hsa-let-7g-5p | 117 |
| hsa-miR-15b-5p | 112 |
| hsa-miR-320e | 112 |
| hsa-miR-221-3p | 109 |
| hsa-let-7d-5p | 108 |
| hsa-miR-16-5p | 106 |
| hsa-miR-424-5p | 102 |
| hsa-miR-3180 | 102 |
| hsa-miR-374a-5p | 101 |
| hsa-miR-15a-5p | 98 |
| hsa-miR-130a-3p | 96 |
| hsa-miR-376a-3p | 92 |
| hsa-miR-199a-5p | 92 |
| hsa-miR-222-3p | 90 |
| hsa-miR-4286 | 89 |
| hsa-miR-4516 | 70 |
| hsa-miR-34a-5p | 65 |
| hsa-miR-1255a | 64 |
| hsa-miR-365a-3p + | 63 |
| hsa-miR-365b-3p | |
| hsa-miR-323a-3p | 60 |
| hsa-let-7c-5p | 59 |
| hsa-miR-27b-3p | 58 |
| hsa-miR-134-3p | 57 |
| hsa-miR-451a | 52 |
| hsa-miR-23b-3p | 50 |
| hsa-miR-423-5p | 50 |
| hsa-miR-28-5p | 49 |
| hsa-miR-125a-5p | 49 |
| hsa-miR-24-3p | 49 |
| hsa-miR-382-5p | 48 |
| hsa-miR-1228-3p | 48 |
| hsa-miR-20a-5p + | 47 |
| hsa-miR-20b- | |
| hsa-let-7e-5p | 45 |
| hsa-miR-18a-5p | 43 |
| hsa-miR-337-5p | 43 |
| hsa-miR-320e | 42 |
| hsa-miR-106a-5p + | 42 |
| hsa-miR-17- | |
| hsa-miR-19b-3p | 41 |
| hsa-miR-140-5p | 40 |
| hsa-let-7f-5p | 39 |
| hsa-miR-323a-5p | 37 |
| hsa-miR-148a-3p | 35 |
| hsa-miR-132-3p | 32 |
| hsa-miR-136-5p | 30 |
| hsa-miR-376c-3p | 30 |
| hsa-miR-379-5p | 30 |
| hsa-miR-26a-5p | 29 |
| hsa-miR-202-3p | 29 |
| hsa-miR-1290 | 29 |
| hsa-miR-154-5p | 27 |
| hsa-miR-214-3p | 27 |
| hsa-miR-377-3p | 27 |
| hsa-miR-381-3p | 25 |
| hsa-miR-188-5p | 25 |
| hsa-miR-26b-5p | 25 |
| hsa-miR-363-3p | 24 |
| hsa-miR-337-3p | 24 |
| hsa-miR-137 | 23 |

### Intramyocardial Injection of Human EVs Reduce Inflammation, Fibrosis and AF in a Rat Model of Sterile Pericarditis

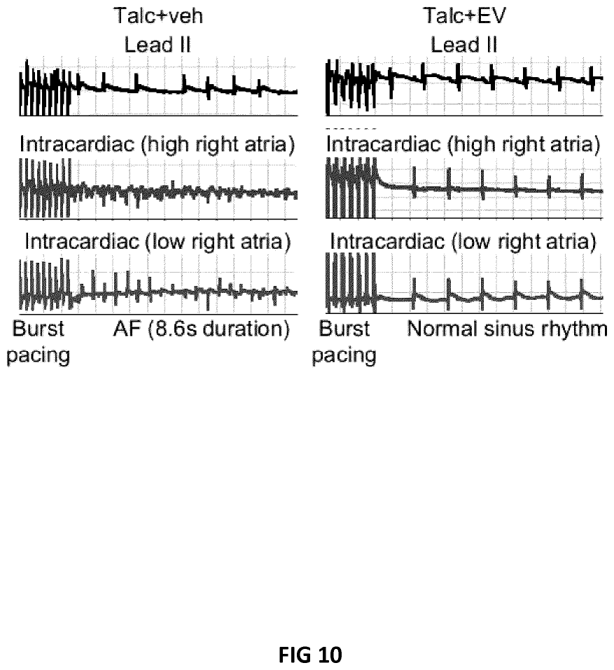

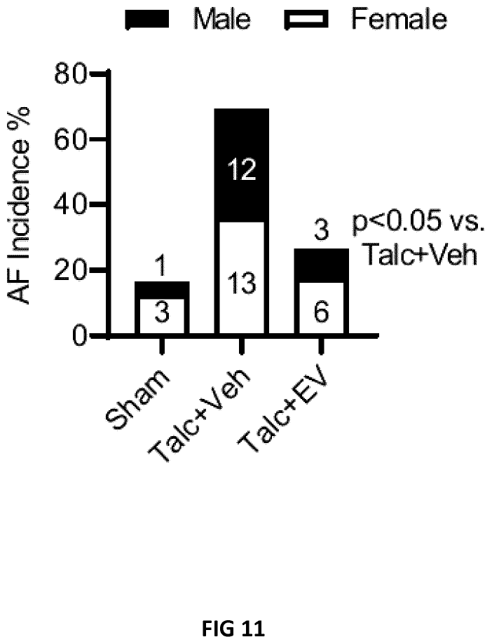

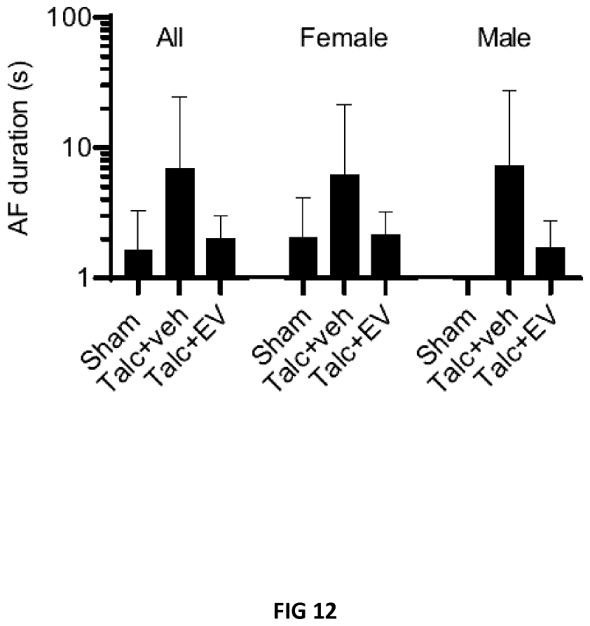

The antiarrhythmic potential of EDC EVs was explored using a rat model of sterile pericarditis (Huang, Chen et al. 2016) whereby all animals underwent open-chest surgery before randomization to epicardial application of talc or no talc. Immediately after epicardial application of talc, animals were randomized again to intra-atrial injection of EVs or vehicle (saline; FIG. 9). As outlined in FIG. 22, all animals survived the initial surgery while 3 vehicle treated animals died because of anesthetic overdose prior to the second procedure. Three days after open-chest surgery, treatment with EVs reduced the probability of inducing AF by 40% (p<0.01 vs. vehicle alone, FIGS. 10-11). Recipient sex did not alter this effect. In animals that experienced AF, neither EV treatment or recipient sex significantly altered AF duration (FIG. 12); although this observation may be attributable to the low number of sham and EV treated animals that experienced AF and the non-parametric distribution of AF duration.

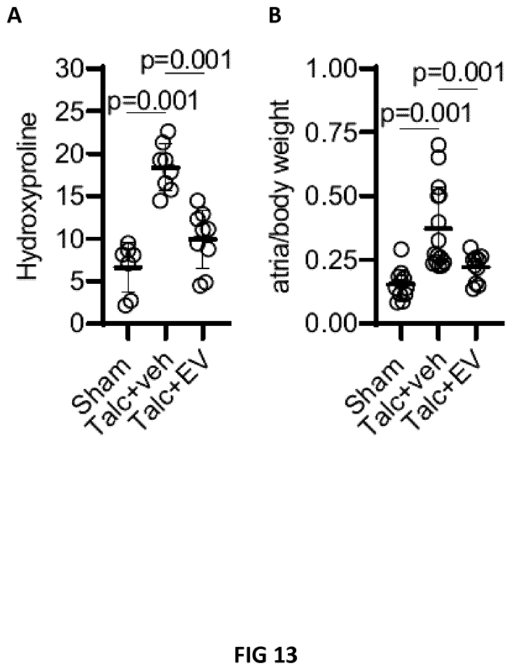

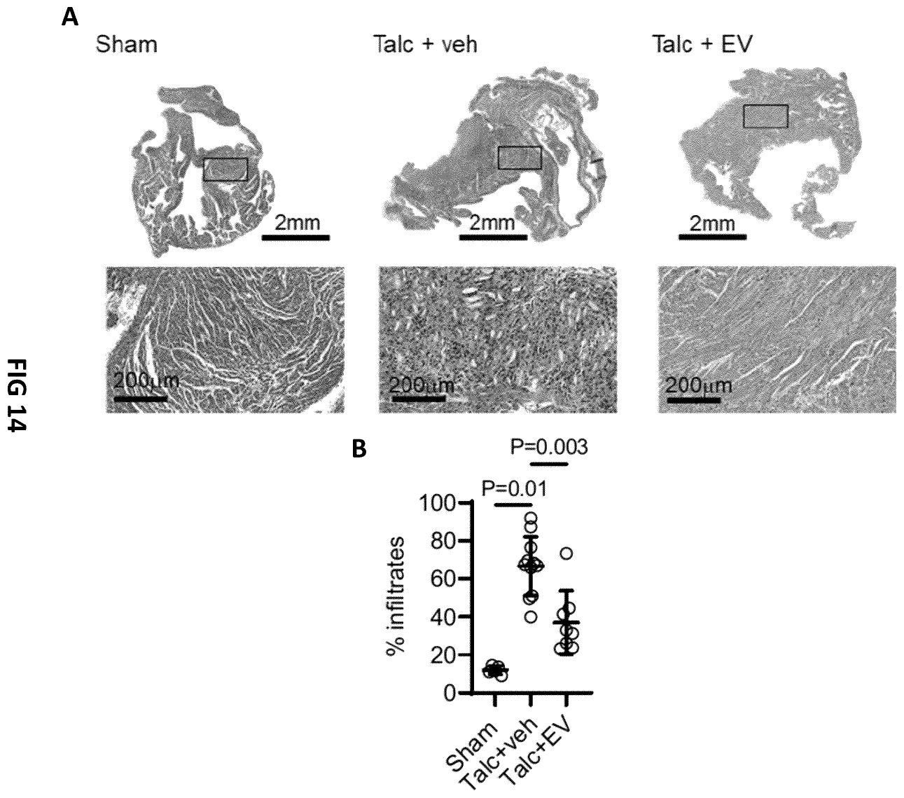

Pericarditis, EV treatment and recipient sex had no effect on electrocardiographic or electrophysiological measures of cardiac function (Table 2). As shown in FIG. 13, EV treatment prevented atrial fibrosis (45±19% reduction in hydroxyproline content, p=0.01 vs. vehicle alone) and enlargement (41±15% reduction in atrial/body weight, p=0.01 vs. vehicle alone).

Table 2 shows the effect of EVs on electrocardiographic and electrophysiological function. AVERP, atrioventricular nodal refractory period; EV, extracellular vesicles; veh, vehicle.

| | RR (ms) | PR (ms) | QRS (ms) | QT (ms) | AVERP (ms) |
|---|---|---|---|---|---|
| Female | | | | | |
| Sham | 174 ± 16 | 49 ± 5 | 19 ± 3 | 93 ± 8 | 77 ± 10 |
| Talc + veh | 163 ± 17 | 48 ± 3 | 18 ± 3 | 87 ± 12 | 76 ± 11 |
| Talc + EV | 169 ± 32 | 49 ± 4 | 17 ± 2 | 94 ± 15 | 80 ± 9 |
| Male | | | | | |
| Sham | 181 ± 18 | 47 ± 3 | 20 ± 2 | 97 ± 8 | 74 ± 13 |
| Talc + veh | 157 ± 12 | 46 ± 4 | 18 ± 2 | 88 ± 9 | 76 ± 10 |
| Talc + EV | 166 ± 16 | 47 ± 4 | 18 ± 3 | 89 ± 11 | 77 ± 10 |

### EVs Prevent Inflammation

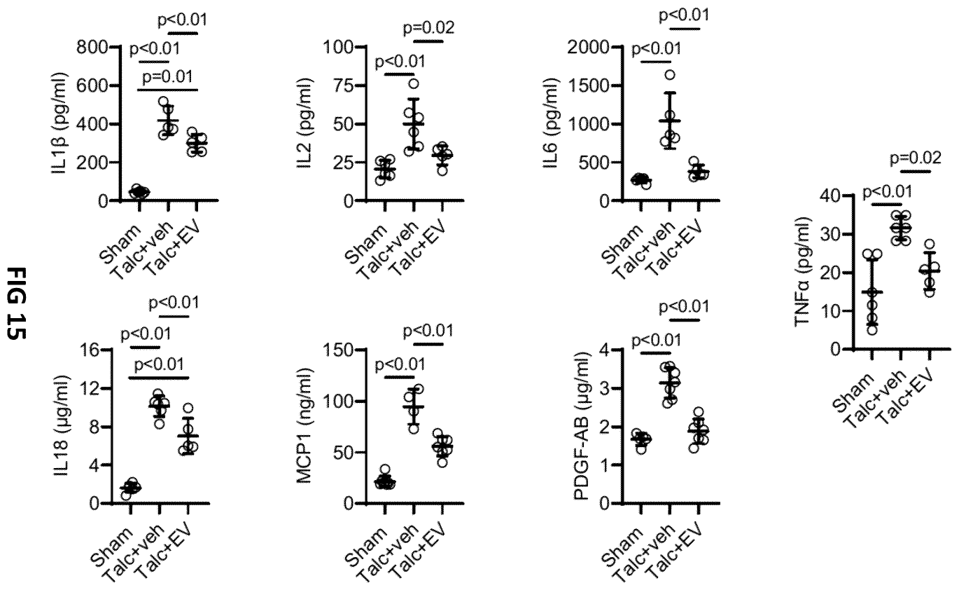

Open chest surgery results in loss of the pericardial mesothelial cells which provokes inflammatory infiltration and an ensuing fibrinous reaction. The influence of human EVs on inflammation was first evaluated using atrial histology of EV treated mice. As shown in FIG. 14, sterile pericarditis markedly increased the inflammatory infiltrate detected in atrial sections (6±1 fold increase, p<0.001 vs. baseline). EV injection attenuated the inflammatory effect of talc as the inflammatory infiltrate was halved (45±24% less; p<0.001 vs. vehicle treatment). This decrease reflected the reduction in pro-inflammatory cytokines found in treated atria (FIG. 15). Sterile pericarditis alone resulted in the prototypical increases of pro-inflammatory cytokines IL1β, IL2, IL6, IL18, MCP1, PDGF-AB and TNFα. Intramyocardial injection of EVs prevented a rise in a number of these cytokines (IL2, IL6, PDGF-AB and TNFα) while attenuating the observed increase in others (IL1β, IL18).

### EVs Directly Prevent the Malignant Transformation of Atrial Fibroblasts

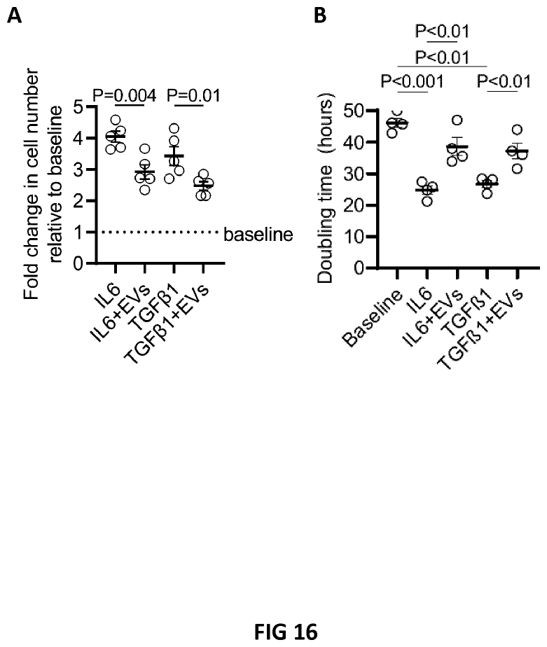

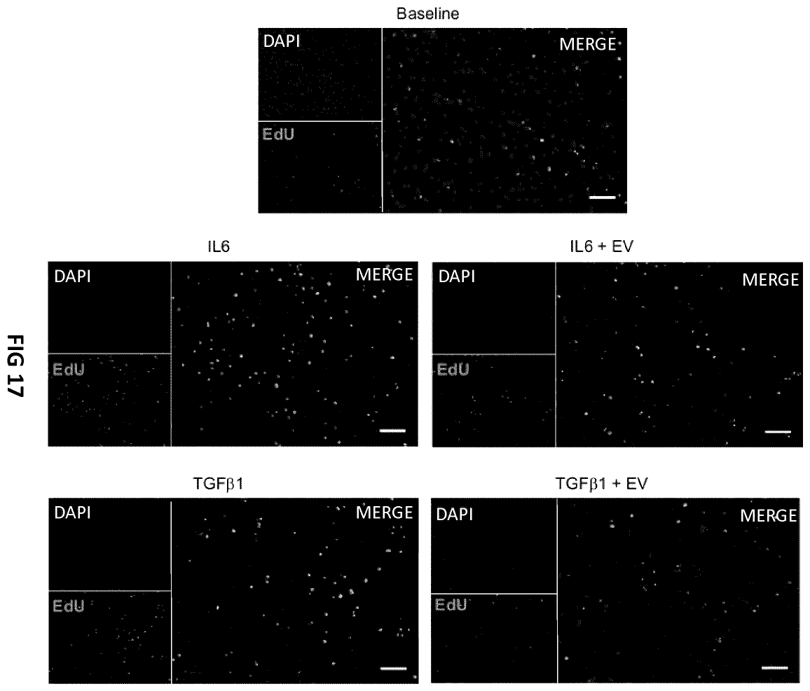

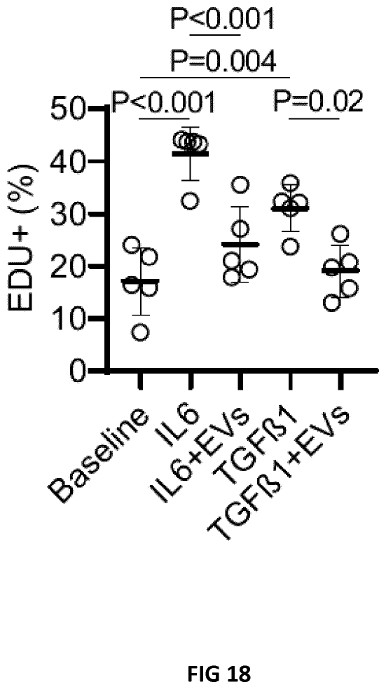

Fibroblasts comprise almost 75% of the cells within the heart (Yue, Xie et al. 2011). When fibroblasts are activated by profibrotic stimuli, they proliferate and differentiate into myofibroblasts which can have adverse effects on atrial structure and electrophysiological function. Given that interfering with atrial fibroblast proliferation reduces fibrosis and AF burden (McRae, Kapoor et al. 2019), we explored the influence of EVs on atrial fibroblast proliferation. POAF was modeled by exposing normal rat atrial fibroblasts to interleukin-6 (IL6) or transforming growth factor beta 1 (TGFβ1) (Narikawa, Umemura et al. 2018). As shown in FIGS. 16-18, IL6 and TGFβ1 increased proliferation as evidenced by increases in manual cell counts and 5-ethynyl-2′-deoxyuridine (EdU) incorporation. Application of EVs at the time of IL6/TGFβ1 exposure reduced fibroblast proliferation back to baseline.

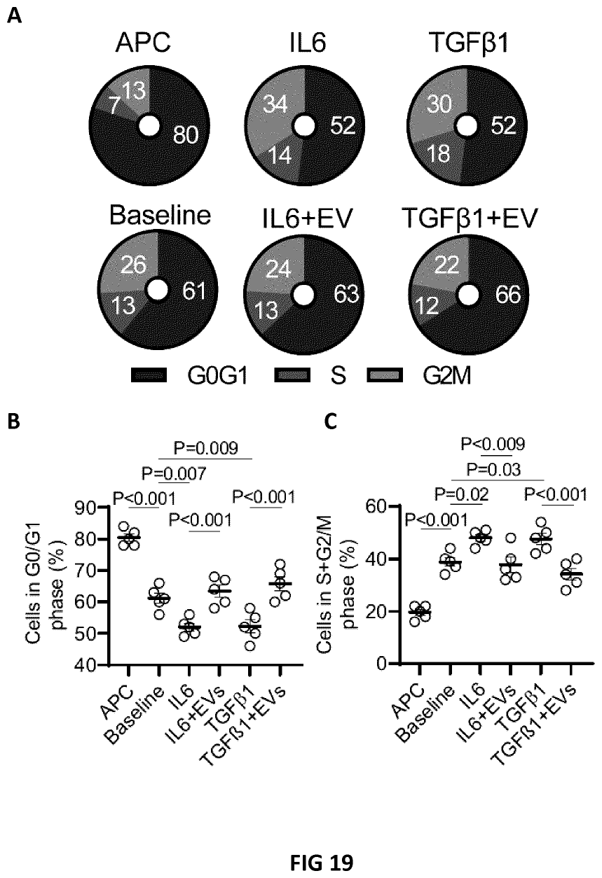

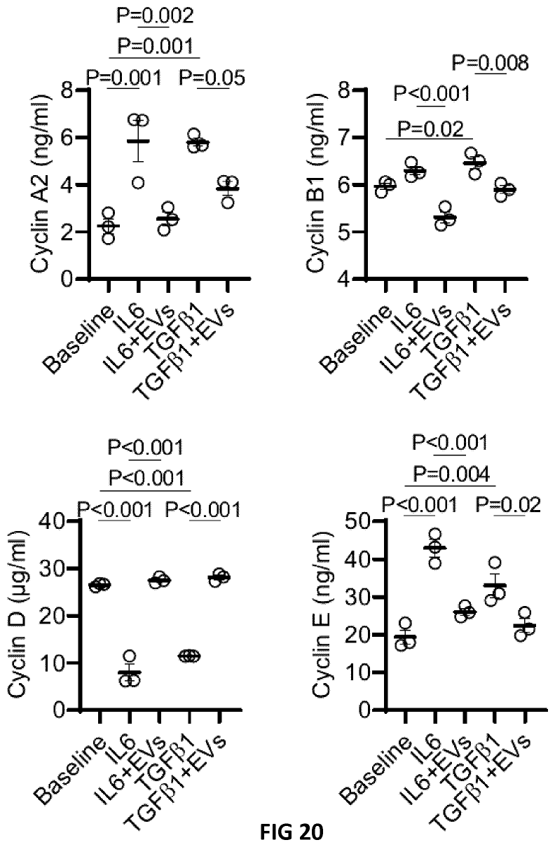

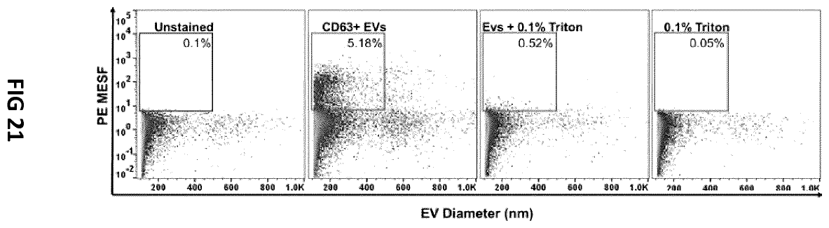

Flow cytometry was then used to profile the effects of EVs on cell cycle kinetics (FIG. 19). Fibroblasts grown in high serum conditions demonstrated progression through the cell cycle (G0/G1, S, and G2/M phases). Anaphase-promoting complex (APC) promoted degradation of cell cycle proteins and cells accumulated in the G0/G1 phase. IL6 and TGFβ1 reduced the proportion of fibroblasts within G0/G1 phase by 15±4% (p=0.004 vs. baseline) and 15±8% (p=0.005 vs. baseline) while increasing the proportion of cells in the proliferative S+G2M phases by 24±7% (p=0.01 vs. baseline) and 24±12% (p=0.01 vs. baseline), respectively. Co-administration of EVs abrogated the effects of each cytokine on the proportional changes in G0/G1 and S+G2M fibroblasts (p=ns vs. baseline). Fibroblast cultures were then interrogated to identify which cyclins within the regulatory machinery underlying cell cycle progression was modified by EV treatment. As shown in FIG. 20, treatment with IL6 or TGFβ1 increased the content of cyclins A2, B1 and E while decreasing the content of cyclin D well below baseline expression. Co-administration with EDC EVs attenuated these effects with cyclins levels often normalizing after treatment. Interestingly, EV treatment decreased expression of cyclin B1 below baseline in a manner consistent with the observed effects on proliferation.

### Example 2

### Materials and Methods

Explant-derived cells were cultured from human atrial appendages in a clinical-grade cell manufacturing facility using serum-free xenogen-free culture conditions. Increasing doses of EVs (10⁶, 10⁷, 10⁸ or 10⁹, n=10-17/group) or vehicle control (n=17) were injected into the atria of middle-age male Sprague-Dawley rats (6 months old) at the time of talc application. A sham control group was included to demonstrate background inducibility (n=12). Three days after surgery, all rats underwent invasive electrophysiological testing prior to sacrifice. Atrial fibrosis was evaluated using hydroxyproline while inflammation was quantified using candidate cytokine profiling.

### Results

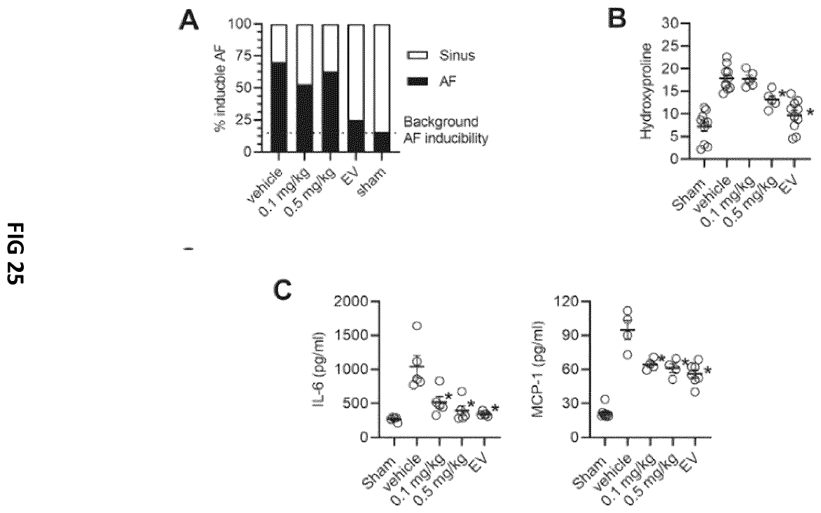

Pericarditis increased the likelihood of inducing AF (p, 0.05 vs. sham). All doses decreased the probability of inducing AF with maximal effects seen after treatment with the highest dose (109, p<0.05 vs. vehicle; FIG. 24A). Pericarditis increased atrial fibrosis while EV treatment limited the effect of pericarditis on atrial fibrosis with maximal effects seen after treatment with 10⁸ or 10⁹ EVs (FIG. 24B). Enzyme-linked immune assay-based analysis of atrial tissue lysate revealed that increasing EV dose was associated with progressive decreases in pro-inflammatory cytokine content (FIG. 24C).

### Example 3

### Materials and Methods

Middle aged female Sprague-Dawley rats (6 months old) were randomized to sham operation (n=17), or induction of sterile pericarditis followed by 0.1 mg/kg daily colchicine (n=15), 0.5 mg/kg daily colchicine (n=15), a single intra-atrial injection of 10⁹ human EVs (n=17) or a single intra-atrial injection of vehicle alone (n=12). Treatment with colchicine began 1 day before pericardiotomy and was continued every day until sacrifice. Explant-Derived Cells were cultured from human atrial appendages in a clinical-grade cell manufacturing facility using serum-free xenogen-free culture conditions. Three days after surgery, all rats underwent invasive electrophysiological testing prior to sacrifice. Atrial fibrosis was evaluated using hydroxyproline while inflammation was quantified using candidate cytokine profiling. Homogenized atrial tissue was used for measurement of cytokines and hydroxyproline.

### Results

Pericarditis increased the likelihood of inducing AF (p<0.05 vs. sham). All doses decreased the probability of inducing AF with maximal effects seen after treatment with the highest dose (10⁹, p<0.05 vs. vehicle; FIG. 25A). Pericarditis increased atrial fibrosis while EV treatment limited the effect of pericarditis on atrial fibrosis with maximal effects seen after treatment with 10⁸ or 10⁹ EVs (FIG. 25B). Enzyme-linked immune assay-based analysis of atrial tissue lysate revealed that increasing EV dose was associated with progressive decreases in pro-inflammatory cytokine content (IL-6 and MCP-1) (FIG. 25C).

### Example 4

### Materials and Methods

Female Sprague Dawley rats were randomized to undergo left coronary artery (LCA) ligation, which results in progressive left ventricular dysfunction, hypocontractility, left atrial dilation, fibrosis, refractoriness prolongation, and AF promotion as compared to sham (Cardin, Guasch et al. 2012). Four weeks later, echocardiographic studies were used to select rats with large infarcts (Cardin, Guasch et al. 2012). Left ventricular regional wall motion were scored in LV short axis for the 6 segments in this view as follows: (1) normal, (2) hypokinesia, (3) akinesia, (4) dyskinesia, and (5) aneurysmal. Wall motion score index (WMSI) is the mean value of all scores. Rats with WMSI less than 1.65 2 weeks after MI were excluded from the study (≈25% of the initial cohort) (Cardin, Guasch et al. 2012). All animals were then randomized to epicardial atrial injection of: 1) vehicle (saline) alone or 2) EVs. 109 EVs were injected at 5 sites within the left atria (Cardin, Guasch et al. 2012). Lab staff were blinded to the treatment received and all analysis were conducted by individuals blinded to group allocation. Group allocations were kept in a separate password protected list for unblinding after analysis of study outcomes is completed.

Eight weeks after LCA ligation, invasive hemodynamics were measured using a standard Millar catheter/recording system ((Tilokee, Latham, et al. 2016), (Jackson, Tilokee et al. 2015)) prior to electrophysiological testing. For electrophysiological testing, an octopolar catheter was inserted into the right atrium via the jugular vein for stimulation and recording of atrial effective refractory periods, Wenckebach cycle length, and corrected sinus node recovery time using twice-threshold (2-ms) square-wave pulses. AF was induced with up to 3 extrastimuli at a cycle length of 100 ms and, if that failed, atrial burst pacing. AF was defined as a rapid and irregular atrial rate (>500 beats per minute) with varying electrogram morphology (Cardin, Guasch et al. 2012). After electrophysiological testing, animals were sacrificed and hearts collected for mechanistic studies.

### Results

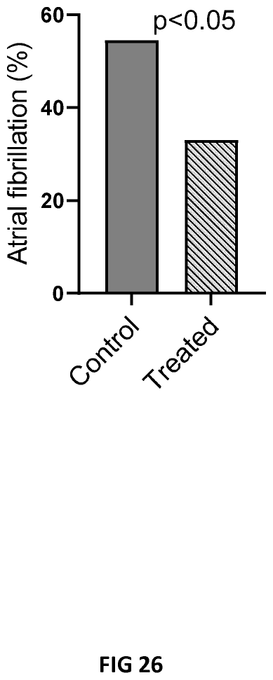

EV treatment after LCA ligation significantly decreased atrial fibrillation in comparison to the control treatment (FIG. 26).

### Example 5

### Materials and Methods

Male C57 mice were fed mice chow and housed under a 12:12-hour light/dark cycle at 21° C. and 50% humidity. All animals had free access to tap water and food. After preoperative, mice were anesthetized with 2-3% isoflurane, intubated, and ventilated. The thorax was shaved and sterilized with 2% w/v chlorhexidine gluconate in 70% v/v isopropyl alcohol. Animals then underwent induction of sterile pericarditis by dusting the atrial surface with sterile talcum powder (Thermo Fischer Scientific) followed by epicardial atrial application of an inert agarose gel (n=6) or an EV impregnated agarose gel (n=6) using a sealed envelope approach. After surgery, animals were placed in a 30 degrees Celsius incubator with supplemental oxygen and moistened food until they returned to a physiological state. Additional doses of buprenorphine were administered 6 and 12 hours postoperatively. A University of Ottawa Animal Care Technician monitored animals twice daily for 2 days after surgery. Investigative staff were blinded to the treatment received and analysis was conducted by individuals blinded to group allocation. Group allocations were kept in a separate password-protected list for unblinding after analysis of the primary study outcome was completed.

Three days after surgery, mice were sacrificed by exsanguination under pentobarbital anesthesia after displaying absence of withdrawal reflex to toe pinch. Atria were fixed and sectioned for histological analysis of fibrosis by staining for Masson Trichrome (Sigma). Left atrial collagen (n=3) was quantified based on hydroxyproline content measurement (K555-100, BioVision).

### Results

EV treatment using a biomaterial gel significantly decreased hydroxyproline content of the atria (54±11 pg vs. 135±12 pg, p=0.01 vs. gel alone) and provided a strong trend towards reduced Masson Trichrome scar (23±5 vs. 35±4 mean % cross-sectional area, p=0.10, n=3) and atrial mass (0.10±0.01 vs. 0.18±0.04 ratio of left atrial to body weight, p=0.06, n=6).

### Example 6

### Material and Methods

Explant-derived cell conditioned media during 48 hours of culture in 1% EV-depleted serum (System Biosciences) at 1% oxygen was used to isolate EVs using ultracentrifugation (Kanda, Alarcon et al. 2018; Villanueva, Michie et al. 2019). EVs were labelled by treating EDCs with the lipophilic carbocyanine dye 1,1′-dioctadecyl-3,3,3′,3′-tetramethylindodicarbocyanine (DiD, Sigma) prior to conditioned media generation.

Cells were seeded in 6-well plates and incubated at 37 degrees Celsius and 5% oxygen. Once at 70-80% confluency, 10⁸ EVs were added to the cells and plates were placed back in the incubator for a set period (10 mins, 1 h, 3 h or 24 h). After incubation, cells were gently washed with PBS, trypsinated and resuspended in final volume of 200 μL PBS. Suspensions were kept on ice at all times until analysis. For each replicate, a control point consisting of cells incubated in the same conditions without addition of EVs was added. Flow cytometric measurements were performed with a Guava® easyCyte Flow Cytometer and data was analyzed with guavaSoft 2.7. The percentage of positive cells was defined using control wells as a baseline.

### Results

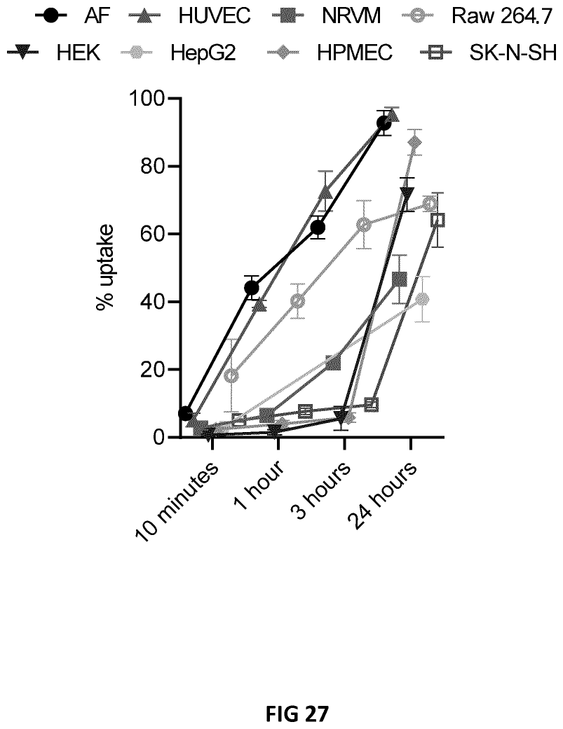

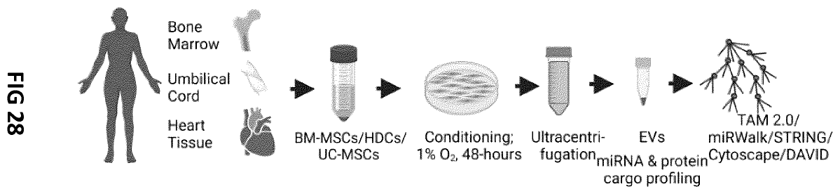

To probe for evidence of differential uptake, we evaluated the rate and extent of DiD labelled EV uptake in atrial fibroblasts (AF), cardiomyocytes, endothelial cells, and macrophages at baseline in vitro (FIG. 27). The potential for off-target effects was probed using brain, kidney, pulmonary, and liver cells. Flow cytometry revealed that mouse macrophages avidly took up DiD labelled EVs with 18±10% staining positive for DiD 10 minutes after exposure. Interestingly, this uptake rapidly plateaued with only 69±2% staining positive for DiD after 24 hours. In contrast, primary rat AFs and HUVECs also demonstrated brisk uptake of DiD labelled EVs (62±8% and 73±10% at 3 hours, respectively) which increased to 96±4% and 95±4% after 24 hours, respectively. Pulmonary microvascular endothelial cells demonstrated negligible EV uptake after 3 hours, but this climbed to 83±8% after 24 hours of continuous exposure. NRVMs demonstrated slow gradual uptake over the first 3 hours (25±4%) which peaked at 44±11% after 24 hours of exposure. Kidney HEK cells showed negligible uptake at 1 h but reaching 72±11% after 24 hours. Liver HepG2 cells demonstrated very little uptake (<1%) over the first hours with less than 46±11% of cells taking up EVs after 24 hours of exposure.

### Example 7

### Materials and Methods

### Cell Culture & EV Isolation

BM-MSCs were isolated from bone marrow samples collected from healthy volunteers enrolled in the Cellular Immunotherapy for Septic Shock (CISS) trial under protocols approved by Ottawa Hospital Research Ethics Board (McIntyre, Stewart, et al. 2018). HDCs were isolated from atrial appendage tissue collected from patients undergoing clinically indicated surgery under protocols approved by the University of Ottawa Heart Institute Research Ethics Board (Mount, Kanda, et al. 2019; Latham, Ye, et al. 2013; Davis, Kizana, et al. 2010). In collaboration with the Centre for Regenerative Therapies in Dresden, UC-MSCs were isolated from primary UC cell isolates under protocols approved by Ottawa Hospital Research Ethics Board (McIntyre, Stewart, et al. 2018). All cell products were manufactured to clinical grade release standards in Biospherix units at the Ottawa Hospital Cell Manufacturing Facility. BM-MSCs were cultured in NutriStem XF media (Sartorius) under 21% oxygen conditions (McIntyre, Stewart, et al. 2018). HDCs were culture in NutriStem XF media at 5% oxygen conditions (Mount, Kanda, et al. 2019). UC-MSCs were cultured in Dulbecco's Modified Eagle Medium (ThermoFisher Scientific) with 10% clinical grade platelet lysate (Mill Creek Life Sciences) at 5% oxygen conditions.

When cells reached 70% confluency, culture media was replaced with condition media (BM-MSC and HDCs: NutriStem XF basal media, UC-MSC: Dulbecco's Modified Eagle Medium with high glucose and 1% platelet lysate). After 48 hours of conditioning at 1% oxygen, media was collected for EV isolation. EVs were isolated using ultracentrifugation (10,000 g×30 minutes and 100,000 g×3 hours) (Villaneuva, Michie, et al. 2019; Kanda, Benavente-Babace, et al. 2020).

Nanoparticle Tracking System The size and concentration of EV preparations were analyzed using NanoSight LM10 equipped with a blue laser (488 nm, 70 mW) with sCMOS camera. Briefly, 1 μL of the final pellet suspension was diluted at 1:1000 in saline and 500 μL was loaded into the sample chamber. Three videos of 60 seconds were recorded for each sample. Data analysis was performed with NTA 3.0 software (Nanosight).

EV proteomic antibody array EV markers were characterized by using proteomic array, as per the manufacturer's recommendations (EXORAY200A; System Biosciences). In brief, 50 μg of EV lysate was incubated with labeling reagent for 30 min followed by incubation with membranes precoated antibodies for 8 known EV markers. After overnight incubation, detection buffer added before membranes were washed and scanned with X-ray imager.

### MicroRNA Expression Assay

MicroRNA was isolated from EVs using the appropriate miRNA isolation kit (Qiagen). One hundred nanograms of miRNA was used for the nCounter miRNA sample preparation reactions. Sample preparation was performed according to the manufacturer's instructions. All hybridization reactions were incubated at 65 degrees Celsius for a minimum of 18 hours. Hybridized probes were purified and counted on the nCounter Prep Station and Digital Analyzer. For each assay, a high-density scan (600 fields of view) was performed. The miRNA count data obtained from Nanostring was analyzed by ROSALIND (https://rosalind.bio/). Background subtraction was performed based on POS_A probe correction factors and normalization was performed using the geometric mean of each code set from the positive control normalization and codeset normalization.

After normalization, fold changes were calculated and comparisons between two EV types were assessed using the ROSALIND t-test method. P-value adjustment was performed using the Benjamini-Hochberg method of estimating false discovery rates (FDR). Log 2 fold change, p-values and normalized Log 2 count data was exported from ROSALIND to construct volcano plots and heatmaps using GraphPad Prism v. 9.1 and RStudio (pheatmap package), respectively. miRNAs were considered differentially expressed with a log 2 fold change ≥ or ≤1.5 and p-value <0.05.

MicroRNA functional enrichment analysis was analyzed using TAM 2.0 (http://www.lirmed.com, (Lu, Shi, et al. 2010; Li, Han, et al. 2018)). The list of mature miRNA names from the normalized ROSALIND data was used as input (overrepresentation, p<0.05). Three functional enrichment categories were extracted (i.e., function, tissue specificity, and transcription factor) to ascribe the functional significance of the miRNA cargo found within EVs. To delineate functional differences pertaining to miRNA cargo across all 3 EV types, we performed enrichment analysis using the list of all differentially expressed miRNAs and all up regulated miRNAs using TAM 2.0.

Target network and enrichment analysis was confined to validated 3′-UTR targets. The list of differentially expressed miRNAs was used as input to miRWalk v3 (http://mirwalk.umm.uni-heidelberg.de/, (Sticht, De La Torre, et al. 2018)) using an interaction probability score of 0.95 (miRTarBase) to obtain the miRNA-mRNA target network. Cytoscape was then used to visualize networks and perform enrichment analysis (Sticht, De La Torre, et al. 2018). Gene ontology enrichment analysis was performed using the Cytoscape plugin BINGO (over representation, hypergeometric test with Benjamini & Hochberg False Discovery Rate correction, p value of 0.05).

### EV Proteomic Analysis

EV isolates containing 25 μg of protein were lysed using a solubilization buffer consisting of 8 M urea, 100 mM 4-(2-hydroxyethyl)-1-piperazineethanesulfonic acid (HEPES), 5% glycerol, and 0.5% n-dodecyl 0-d-maltoside (DDS). Samples were reduced using tris(2-carboxyethyl) phosphine (1.6 mM) and then alkylated with iodoacetamide (8 mM) for 55 min at room temperature. Proteins were digested using 0.45 μg of trypsin/Lys-C solution (Promega) at room temperature for 20 hours. Two microliters of formic acid was then added to samples which were then desalted using C18 TopTips (Glygen) columns, and finally vacuum dried. Five micrograms of protein were then analyzed by Orbitrap Fusion mass spectrometry (Thermo Fisher Scientific) (Risha, Minic, et al 2020). Peptides were separated by an in-house packed column (Polymicro Technology) using a water/acetonitrile/0.1% formic acid gradient. Samples were loaded onto the column for 105 min at a flow rate of 0.30 μL/min. Peptides were separated using successive rounds of acetonitrile at concentrations 2-90% in a step wise manner for every 10 minutes. Peptides were eluted and sprayed into a mass spectrometer using positive electrospray ionization at an ion source. Peptides mass spectrometry spectra (m/z 350-2000) were acquired at a resolution of 60,000. Precursor ions were filtered according to monoisotopic precursor selection, and dynamic exclusion (30 seconds±10 ppm window). Fragmentation was performed with collision-induced dissociation in the linear ion trap. Precursors were isolated using a 2 m/z isolation window and fragmented with a normalized collision energy of 35%.

Differential protein expression analysis was performed using Perseus (https://maxquant.net/, Tyanova, Temu, et al. 2016). Label-free quantitation values were log 2 transformed for visual inspection using histogram distribution plots for each sample. Proteins identified in at least 2 of the 3 replicates were considered for analysis. A two tailed Student's t-test with permutation-based FDR was used to calculate statistical significance between two EV types. The log 2 fold difference, p-values and log 2 label-free quantitation values were used to make volcano plots and heatmaps using GraphPad Prism v. 9.1 and RStudio (pheatmap package), respectively. The proteins were considered differentially expressed with a p-value <0.05.

Functional annotations and pathway enrichment analysis of the whole proteome and differentially expressed proteins was performed using the Database for Annotation, Visualization, and Integrated Discovery (DAVID, v6.8, https://david.ncifcrf.gov/, Sherman, Hao, et al. 2022; da Huang, Sherman, et al. 2009) with Homo sapiens proteome as a background. Fisher's exact test with multiple testing by the Benjamini-Hochberg method with an adjusted p-value of 0.05 was used to extract significantly enriched terms. The top 10 functional GO terms of biological process, cellular component, molecular function and Kyoto Encyclopedia of Genes and Genomes (KEGG) pathways were extracted with enrichment analysis performed on all protein and upregulated regulated proteins separately.

Protein-protein interactions analysis of differentially expressed proteins was performed using Search Tool for the Retrieval of Interacting Genes (STRING, vii.5, https://string-db.org/cgi/input.pl, Szklarczyk, Gable, et al. 2019) with a medium confidence score of 0.4. Cytoscape was then used to perform cluster and enrichment analysis. The Cytoscape plugin MCODE was used to perform cluster and enrichment analysis (degree cut off=2, cluster finding=haircut, node score cut off=0.2, K-core=2, Maximum depth=100) while the cytoHubb plugin was used to identify hub genes.

### Statistical Analysis

All statistical tests and graphical depictions of data are defined within the respective methods sections. Unless otherwise stated, all data are presented as mean±standard error of the mean. To determine if differences existed in EV size and concentration between cell types, the data was analyzed by a one-way analysis of variance (ANOVA; GraphPad Prism v. 9.1) with post hoc testing using Tukey's Multiple Comparisons test. A final value of P<0.05 was considered significant for all analyses.

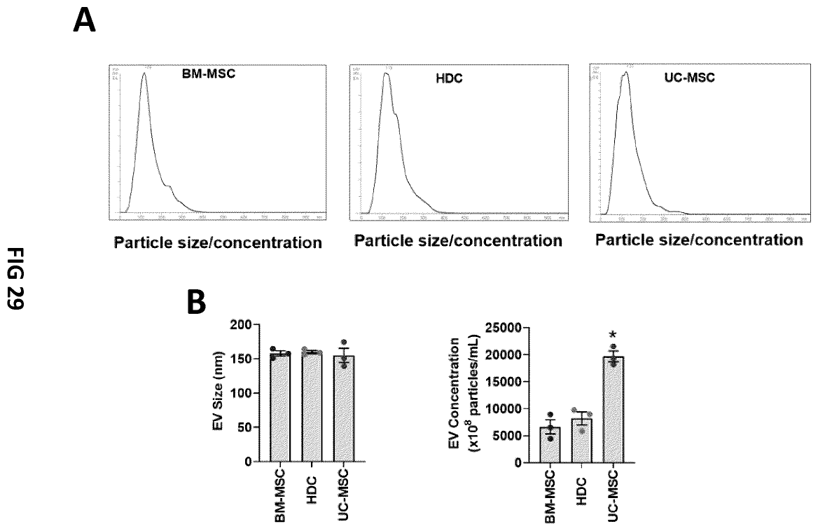

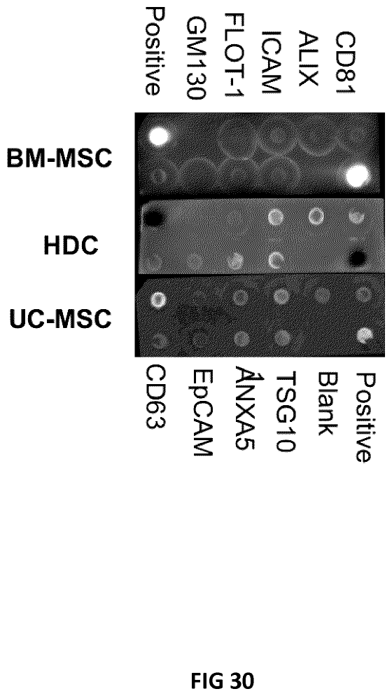

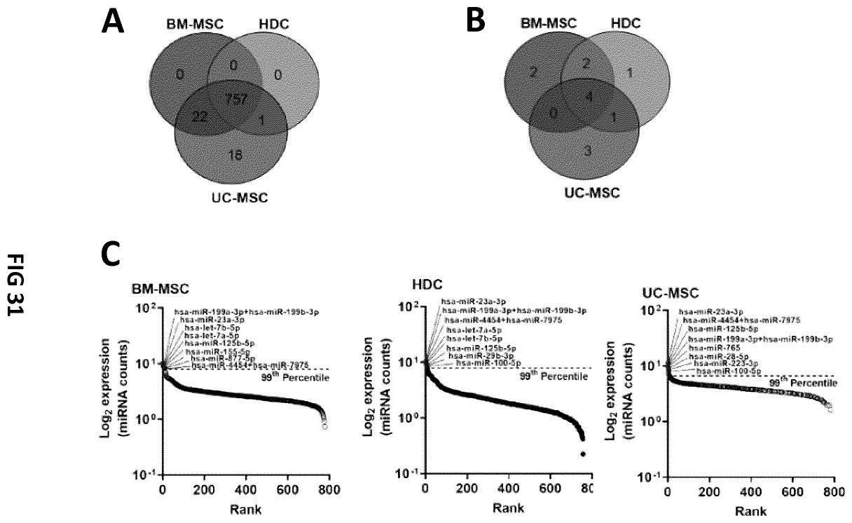

Results Cell culture and EV characterization A schematic of the experimental methodology is shown in FIG. 28. All three cell products were manufactured to clinical release standards, as previously described (McIntyre, Stewart, et al. 2018; Schlosser, Wang, et al. 2019; Vaka, Khan, et al. 2022). These cell isolates were previously characterized for their surface marker identity (Vaka, Khan, et al. 2022) and tri-lineage differentiation (English, Fergusson, el al. 2021), hence cell characterization was not repeated in the current study. EVs were isolated from conditioned media collected from cultured cells using differential ultracentrifugation. The size of EVs isolated from all 3 cell lines were representative of accepted definitions for EV identity (Lbtvall, Hill, et al. 2014) with no differences attributable to producer cell line (FIG. 29A-B). While BM-MSCs and HDCs produced similar amounts of EVs, UC-MSCs yielded 2-fold more EVs (p<0.05 vs. BM-MSCs or HDCs). All 3 EV preparations expressed common EV surface markers (ALIX, ANXAS, CD81, CD63, EPCAM, FLOT-1, TSG101, ICAM) without evidence for cellular contamination (GM130; FIG. 30).

EV miRNA and Protein Cargo Profiling within Bone Marrow, Heart, and Umbilical Cord EVs

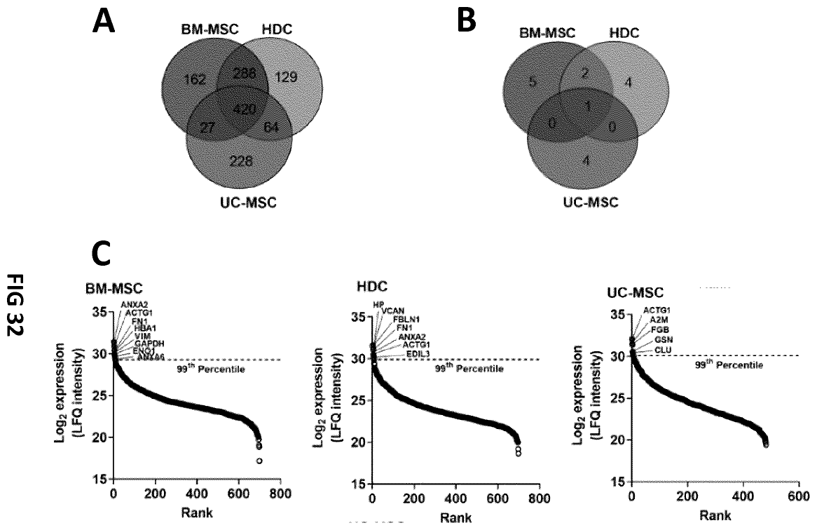

As shown in FIG. 31A, C, 757 miRNAs were commonly expressed by all 3 EVs. Interestingly, UC-MSCs were found to express the most distinct EVs with 18 unique miRNAs. Within the highly expressed miRNAs (i.e., expression above 99th percentile), 4 miRNAs were found to be commonly expressed among all 3 EVs (miR-199a+miR-199b, miR-23a, miR-4454+miR-7975, miR-125b-5p). Two miRNAs (let-7a, let-7b) were shared between BM-MSC and HDC EVs while 1 miRNA (miR-100) was found in both HDC and UC-MSC EVs (FIG. 31B).

As shown in FIG. 32A, C, 420 proteins were expressed in all 3 cell types. In contrast to the few miRNAs uniquely expressed, all 3 EVs were found to contain several unique proteins (i.e., 100+) with the greatest number of shared proteins found in BM-MSCs and HDCs. UC-MSCs expressed the greatest number of unique proteins. When ranked as highly expressed proteins, only ACTG1 protein was shared above the above 99th percentile in EVs from 3 cell types while ANXA2 and FN1 was shared between BM-MSC and HDC EVs (FIG. 32B).

Differential Expression of microRNAs or Proteins within Bone Marrow, Heart, and Umbilical Cord EVs

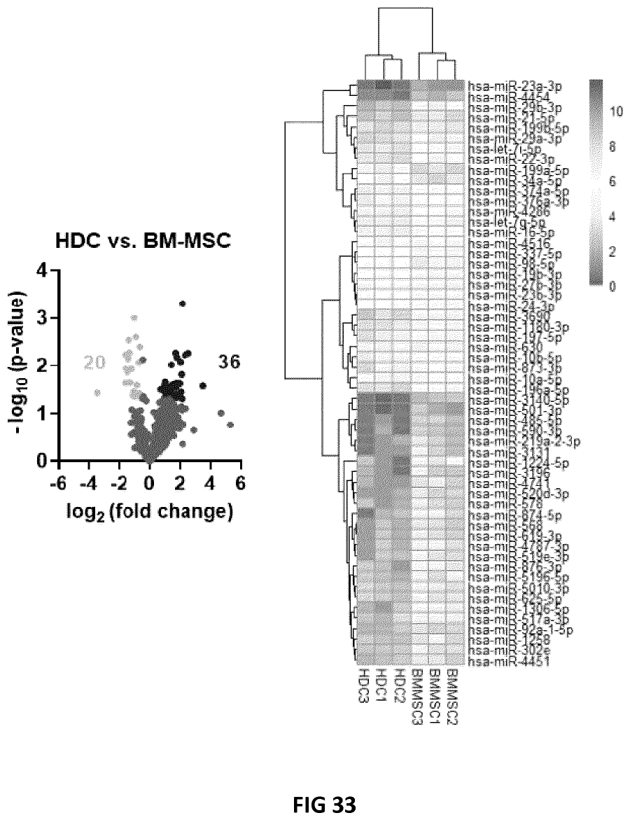

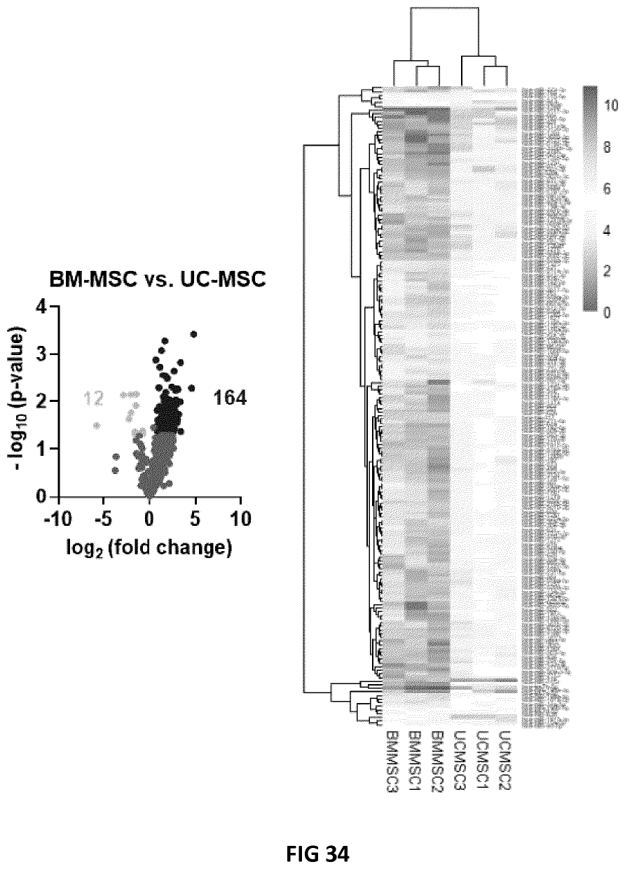

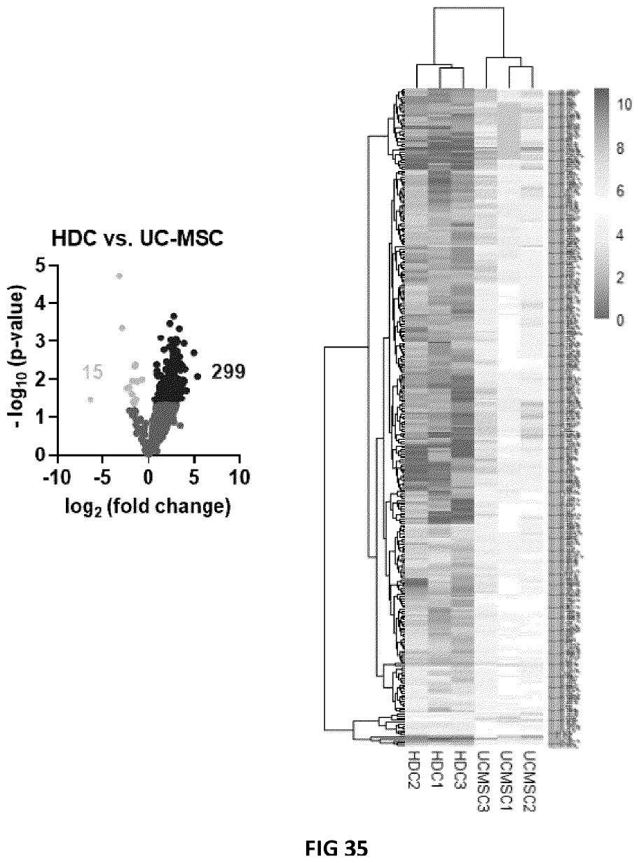

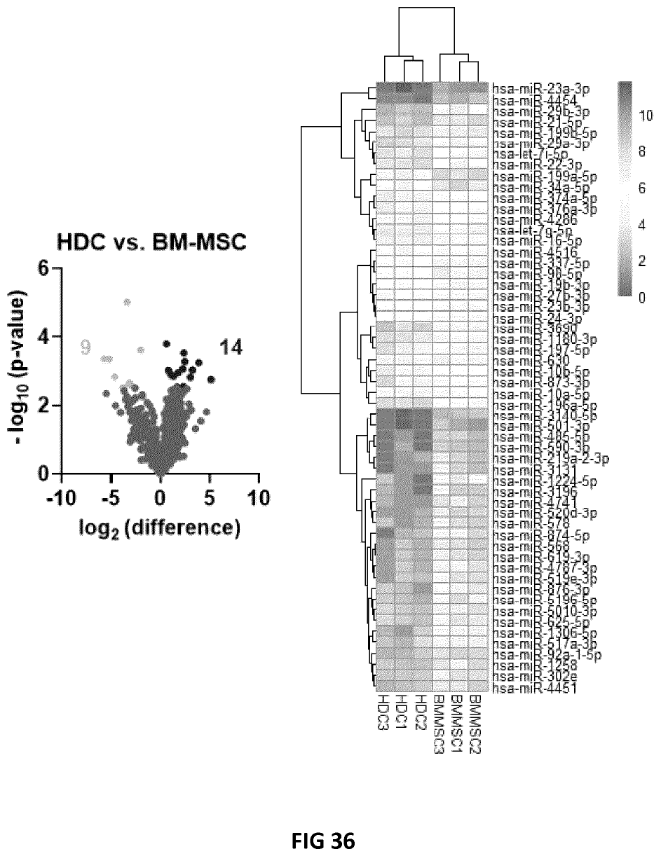

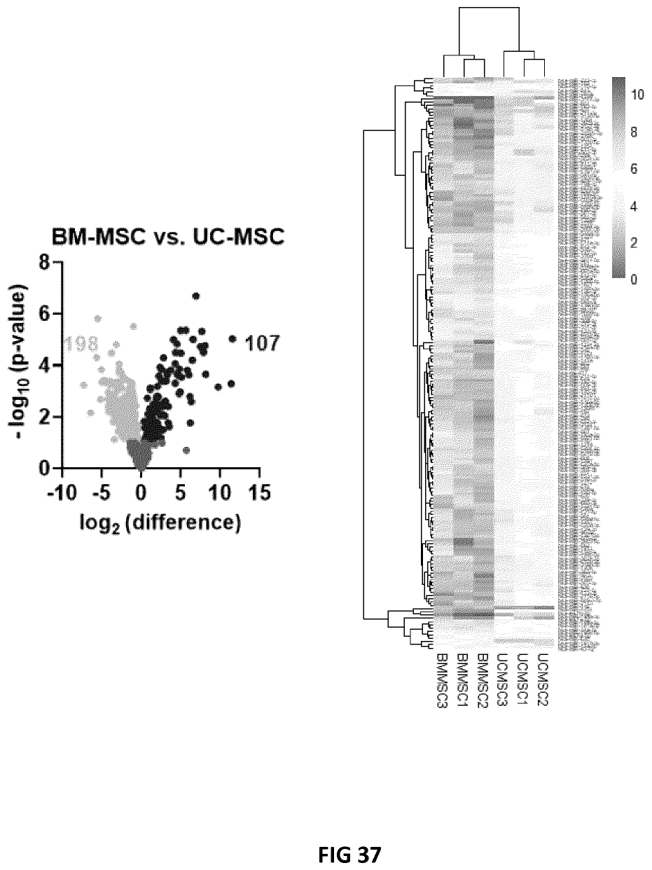

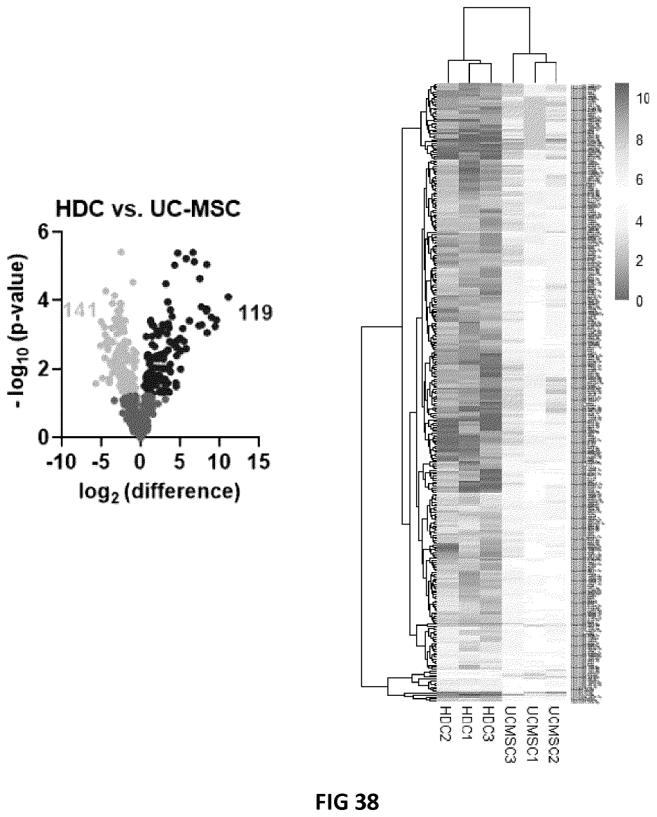

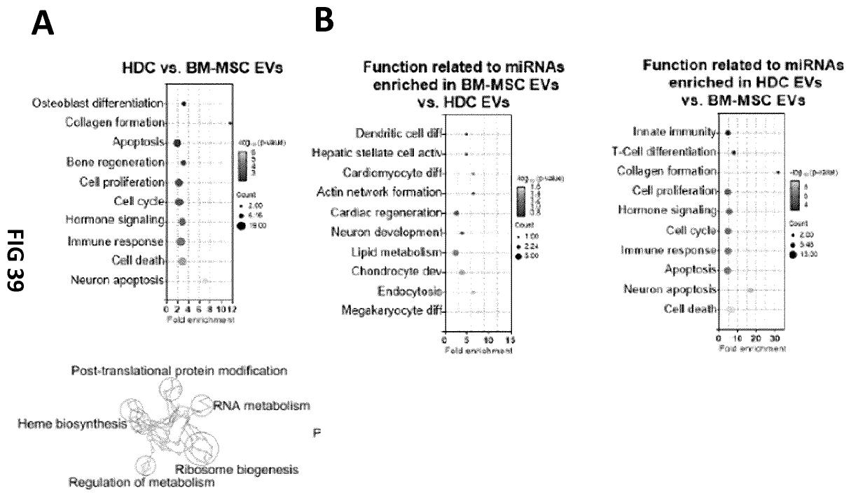

To probe the miRNA stoichiometry for differences in expression patterns, we compared differential miRNA expression using a 1.5-fold Log 2 threshold (FIGS. 33-35 and Table 3-5). Expression of miRNA in BM-MSC and HDC EVs was largely similar with only 56 differentially regulated transcripts. In contrast, UC-MSC EV expression was markedly different with 176 and 314 differentially expressed miRNAs as compared to BM-MSC and HDC EVs, respectively. Analysis of protein expression showed a similar pattern with only 23 proteins showing differential expression when BM-MSC EVs were compared to HDC EVs (FIGS. 36-38 and Table 6-8). UC-MSC EVs had 305 and 260 differential expressed proteins when compared to BM-MSC and HDC EVs, respectively.

Functional Analysis of miRNA Expression within Bone Marrow, Heart, and Umbilical Cord EVs

As shown in FIGS. 39, 41, 43, HDC EVs expressed miRNA transcripts implicated in apoptosis, collagen formation, osteoblast differentiation, and cell proliferation when compared to EVs from BM-MSCs. GO enrichment analysis demonstrated that miRNA-mRNA targets were significantly enriched in pigment biosynthesis, mRNA catabolism and RNA processing. MicroRNAs highly expressed in HDC EVs were associated with differentiation, cardiac regeneration, and differentiation while miRNAs highly expressed in BM-MSC EVs were involved in collagen formation, cellular proliferation, and cell death. EVs from BM-MSCs were enriched in transcripts implicated in apoptosis, hematopoiesis, and retinal development when compared to EVs from UC-MSCs. GO analysis of transcripts from BM-MSCs EVs suggest involvement in glucose metabolism, protein transport, RNA processing and vacuole transport. MicroRNAs upregulated in BM-MSC EVs were involved in apoptosis, cell migration, and osteogenesis while miRNAs enriched within UC-MSCs were involved in aging, cardiac regeneration, and proliferation.

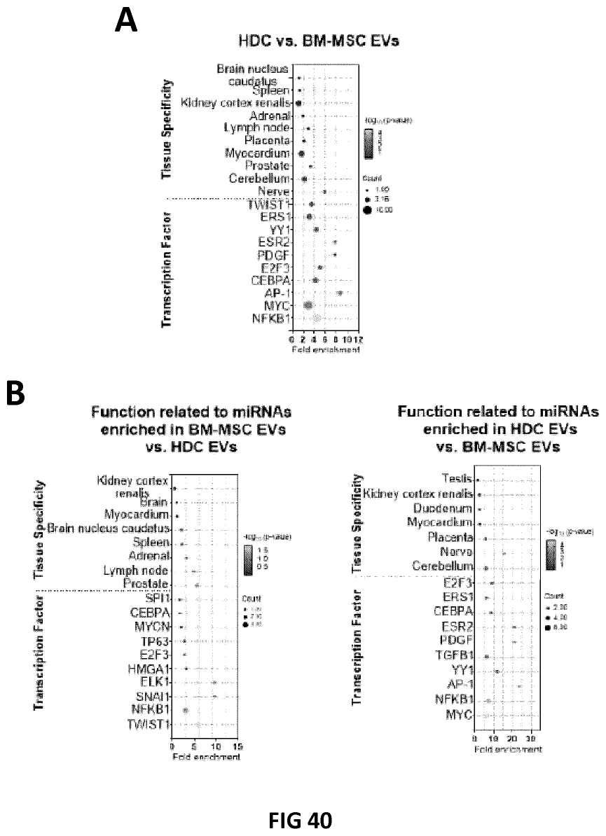

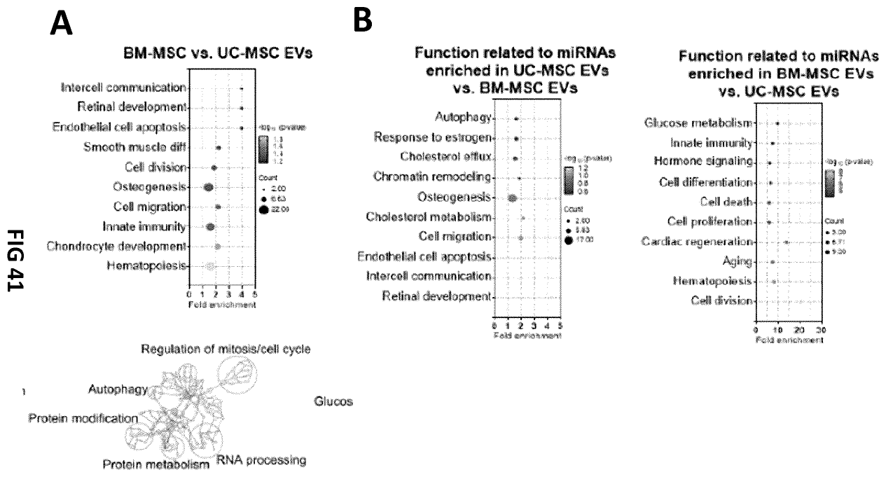

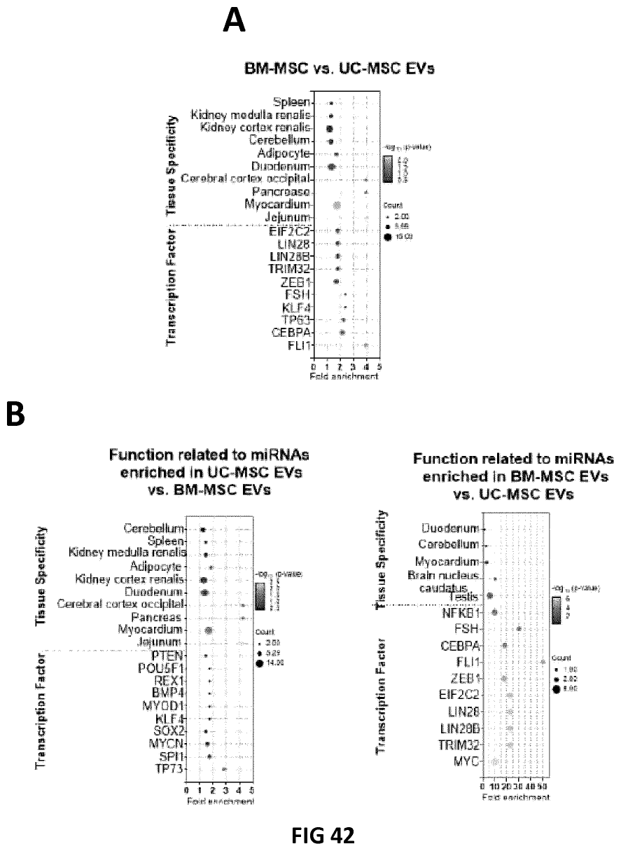

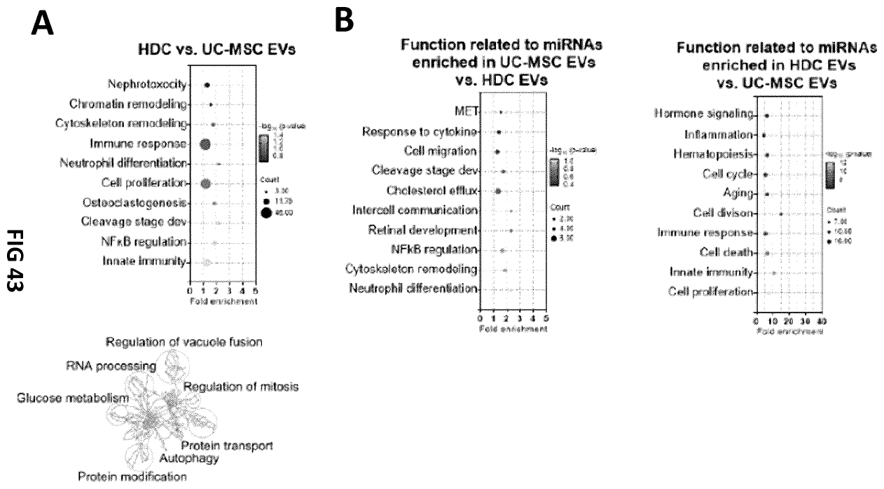

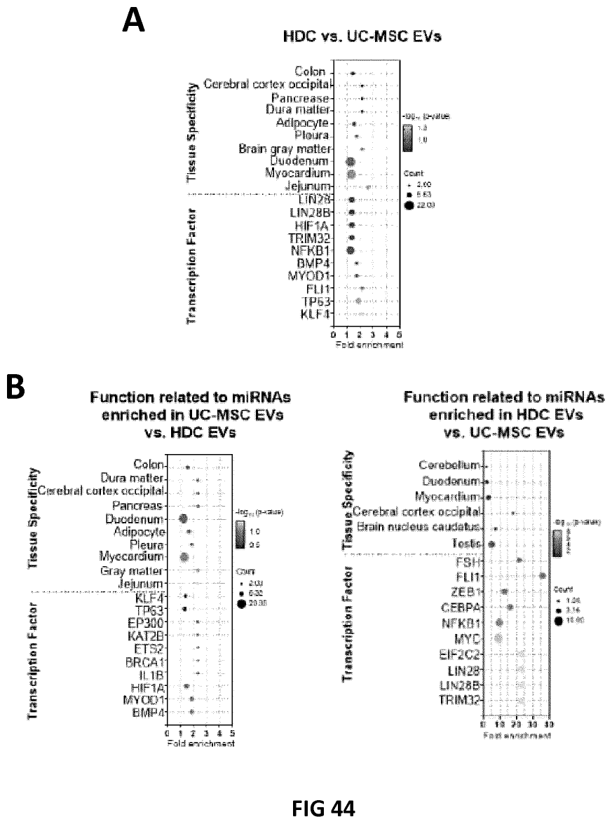

When HDC EVs were compared to UC-MSCs EVs, miRNAs were implicated in development, immune regulation, and proliferation. GO analysis of transcripts from HDC EVs were implicated in glucose metabolism, protein transport, RNA processing and vacuole transport. Highly expressed miRNAs in HDC EVs were involved in mesenchymal to epithelial transition, regulation of NFκB, and development while highly expressed miRNAs in UC-MSCs were involved in aging, inflammation, and proliferation. The downstream transcription factors and tissue associated with these functions for EVs from all 3 producer cell lines are shown in FIG. 40, 42, 44.

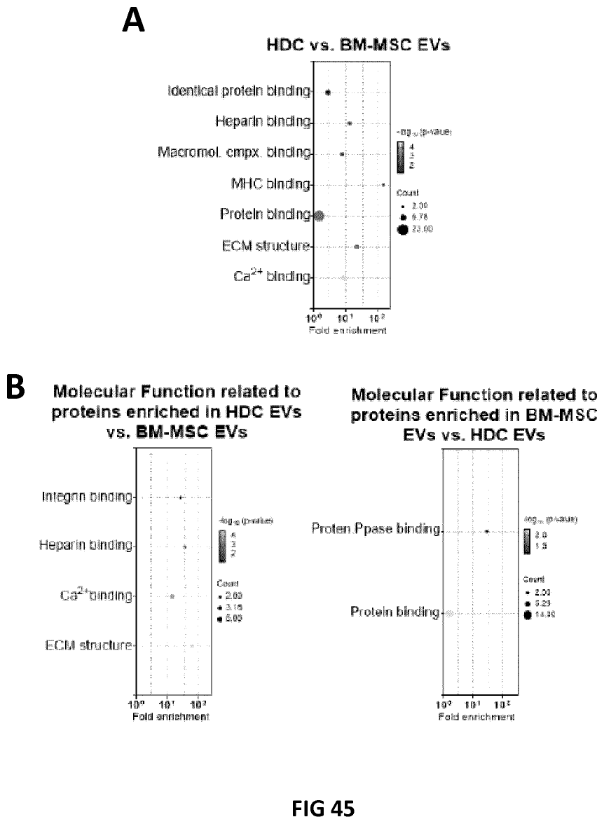

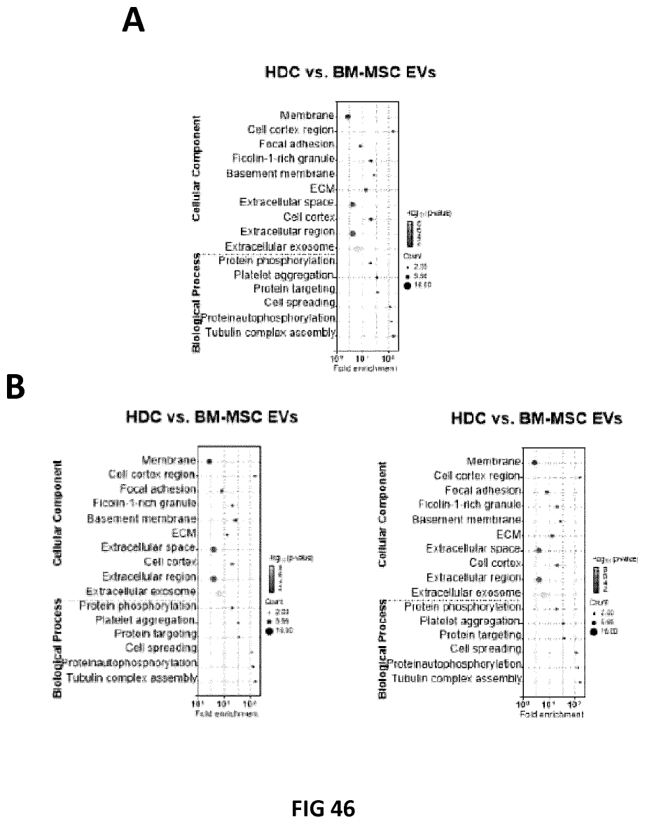

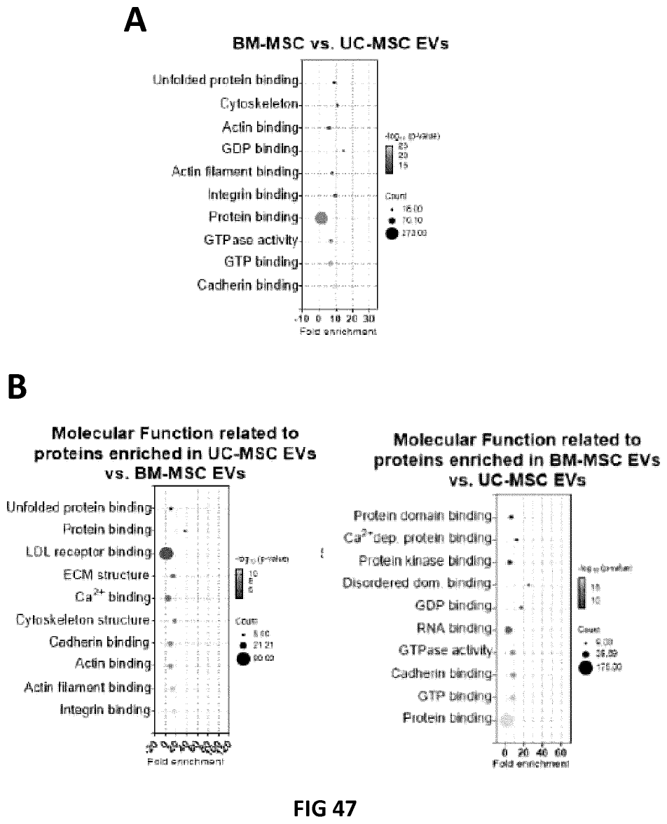

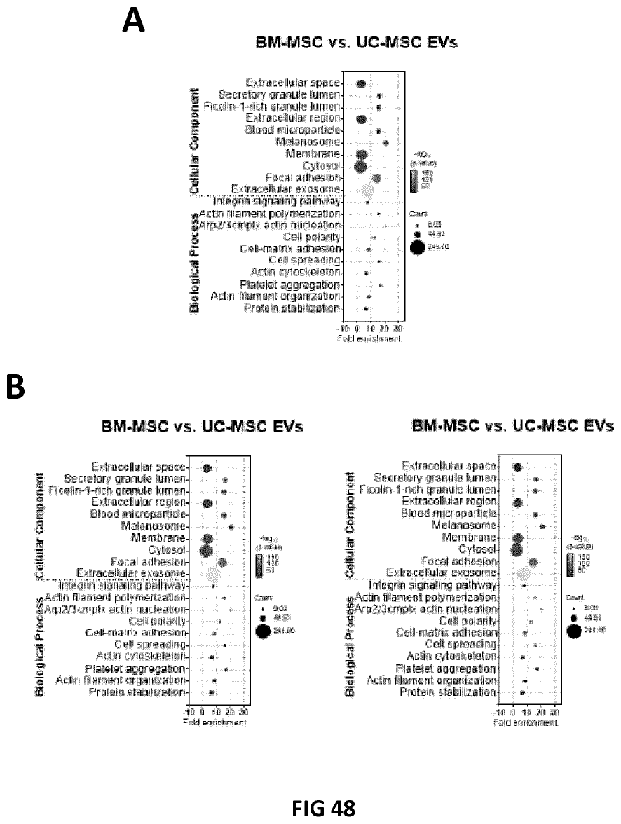

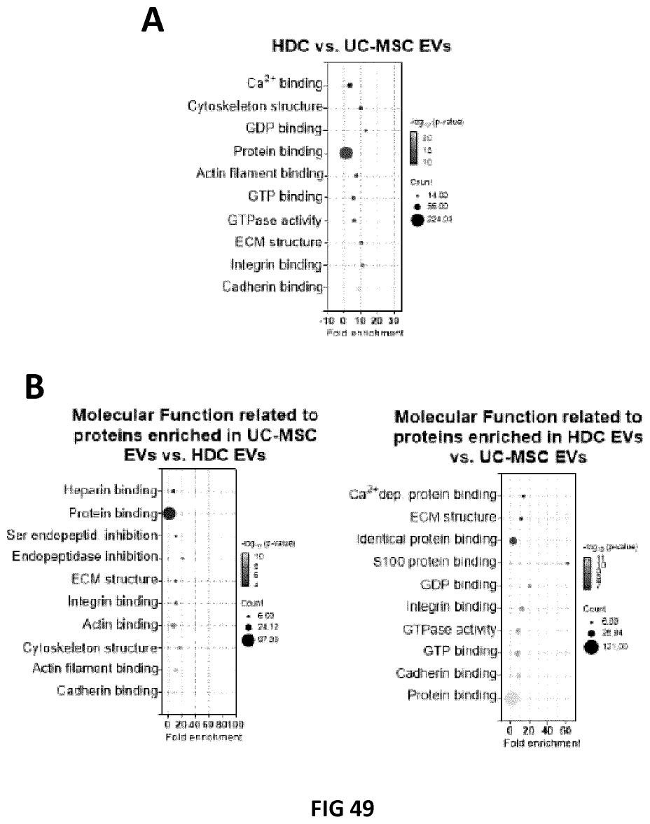

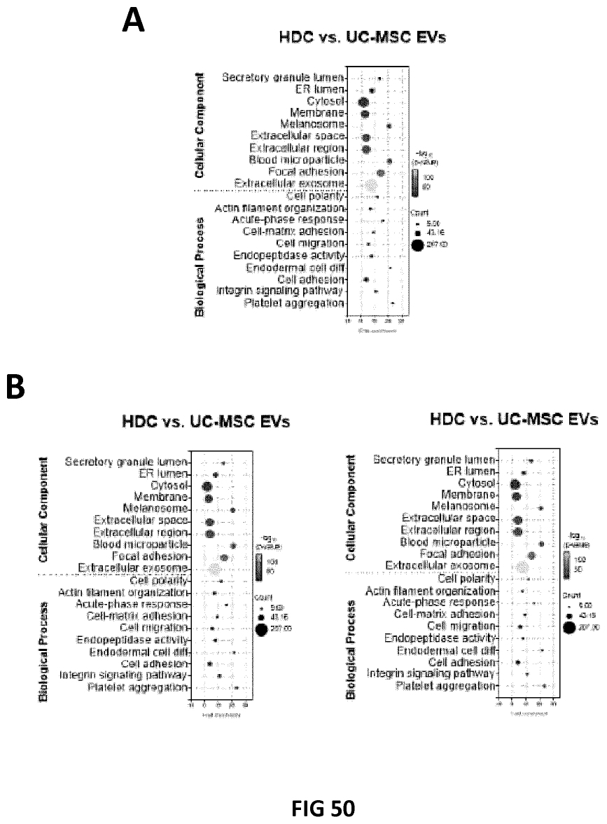

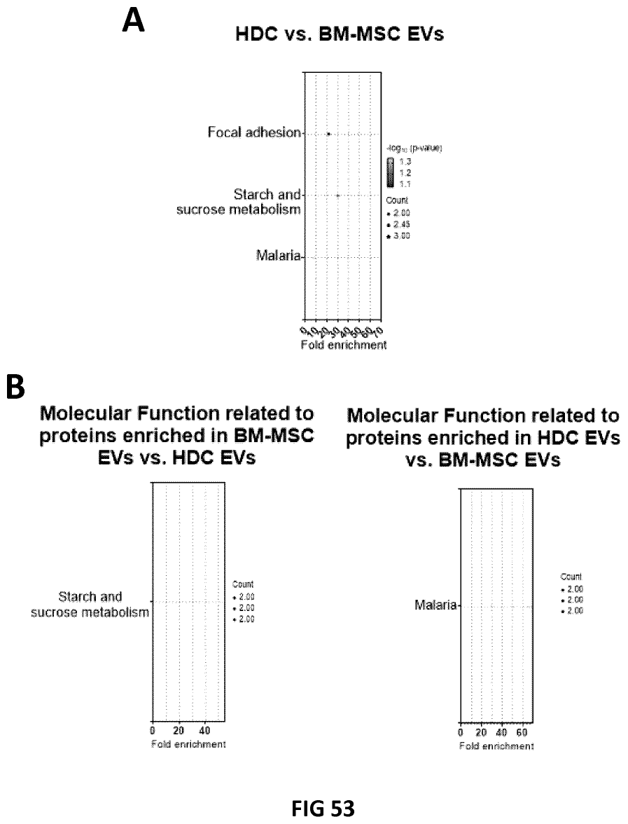

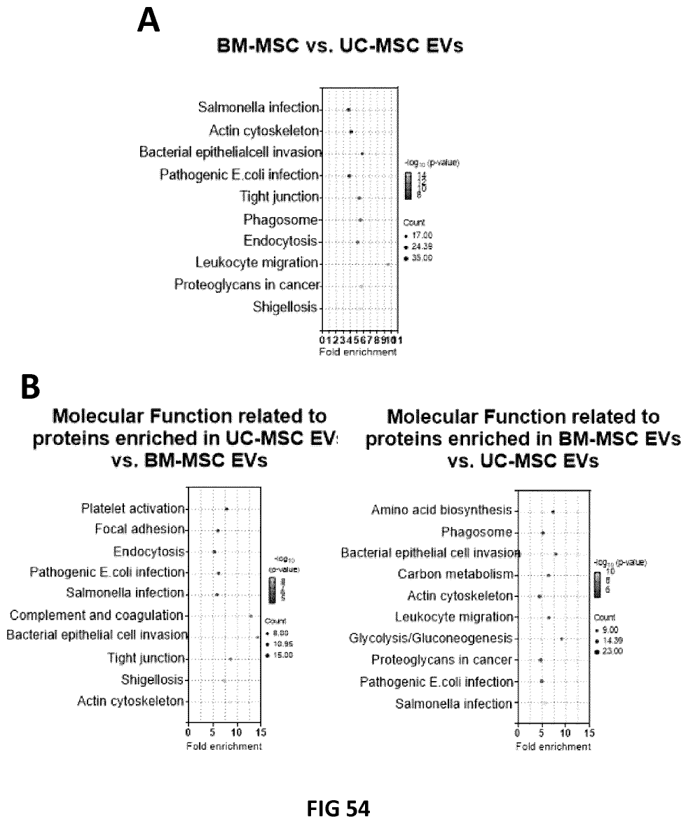

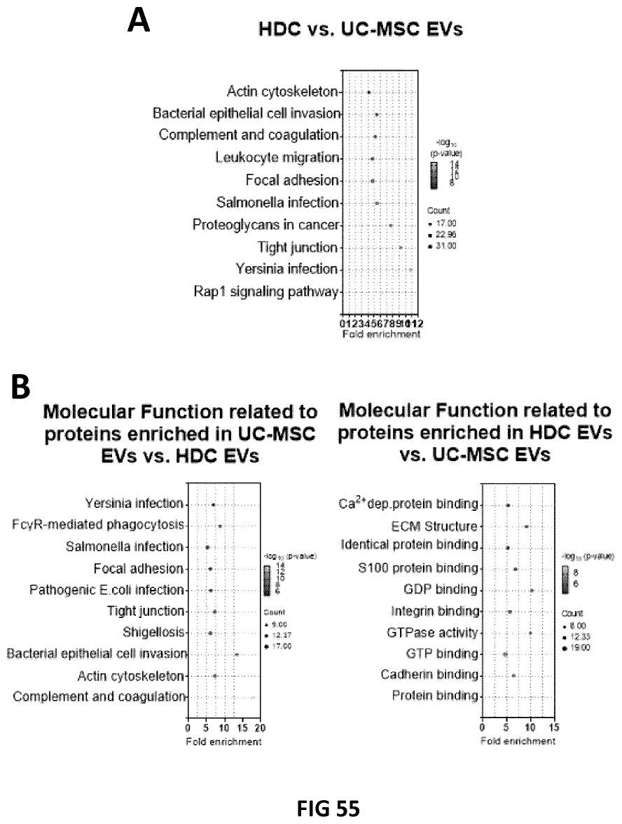

Functional Analysis of Protein Expression in Bone Marrow, Heart, and Umbilical Cord EVs Enrichment analysis of the protein cargo within EVs revealed several terms related to biological processes, cellular component, molecular function and KEGG pathways for each cell source. The top 10 biological processes, cellular components and KEGG pathways are shown in FIGS. 46, 48, 50, and 53-55. Many of the top terms were shared and related to EV biology or cellular binding (i.e., cell adhesion, extracellular exosome, focal adhesion, cadherin binding, and protein binding). When EVs from different cell types were compared, each producer cell line imparted several unique terms with the greatest degree of homology shared between BM-MSC and HDC EVs. When HDC EVs were compared to BM-MSC EVs, functional pathways were largely involved in calcium ion binding, cell binding and extracellular matrix assembly (FIG. 45). Proteins over-expressed in EVs produced by BM-MSCs were more apt to be implicated in heparin or integrin binding. When BM-MSC EVs were compared to UC-MSC EVs, differentially expressed proteins were involved in actin organization, cadherin binding, and cellular adhesion (FIG. 47). Proteins highly expressed within BM-MSC EVs were involved in protein+RNA binding while UC-MSC EVs highly expressed proteins in extracellular matrix binding/structure and LDL receptor biology. When HDC EVs were compared to UC-MSC EVs, proteins were implicated in calcium and protein binding (FIG. 49).

### Protein-Protein Interactions Imparted by Bone Marrow, Heart, and Umbilical Cord EVs

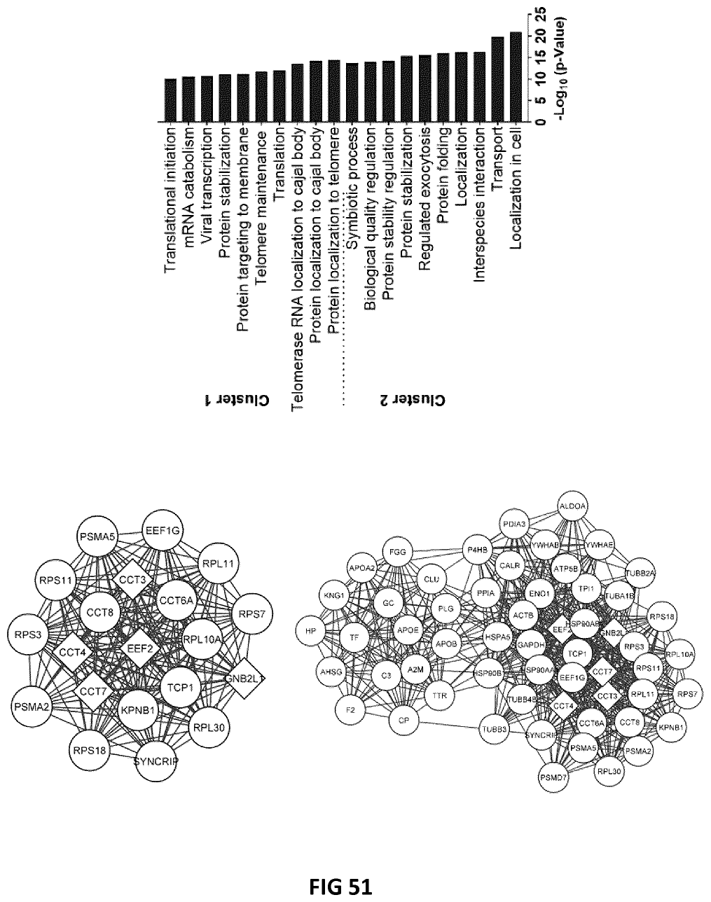

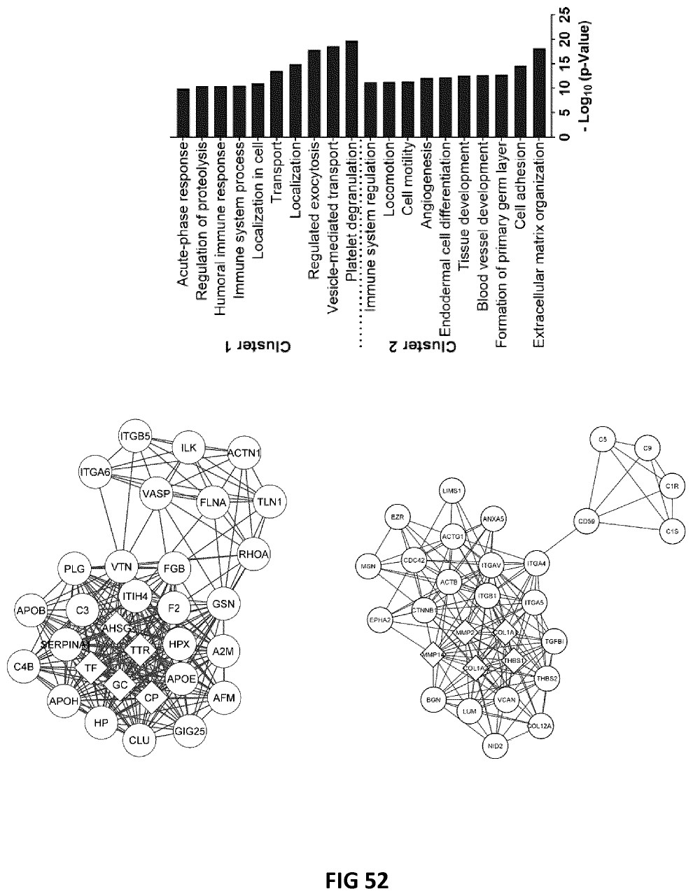

Proteins regulate biological processes through functional or physical interactions with other proteins. Using protein-protein interaction network analysis, we probed for potential interactions between differentially expressed proteins. As shown in FIGS. 51 and 52, this analysis revealed several nodes and interaction pairs with the highest degree of homology shared between HDC and BM-MSC EVs. Key nodes were then used to extract hub genes for cluster analysis which yielded 1 cluster from the HDC vs. BM-MSC EV network, 9 clusters from the BM-MSC vs UC-MSC EV network, and 7 clusters from the HDC vs. UC-MSC EV network.

Using a topological scoring system (Bandettini, Kellman, et al. 2012), we found the HDC vs. BM-MSC EV network did not contain significant clusters, indicating a high degree of homology. Two significant clusters were found within the comparison between BM-MSC and UC-MSC EVs (topological score=19.36, 20 nodes, 184 interaction pairs). Analysis of cluster 1 revealed hub genes related to chaperon containing proteins (CCT3, degree=32; CCT7, degree=34; CCT8, degree=26) and ribosomal protein subunits (RPS3, degree=28; RPL11, degree=25; FIG. 51). String enrichment analysis indicated the top biological processes were related to protein localization to telomeres (GO:1904851), protein localization to cajal bodies (GO:1904871), and telomerase localization to cajal bodies (GO:1904874). Cluster 2 contained 57 nodes and 668 interaction pairs with the top 5 hub genes being related to chaperon containing proteins (CCT3, degree=32; CCT4, degree=33; CCT7, degree=34), nucleotide binding protein (GNB2L1, degree=34) and translation elongation factor (EEFF2, degree=39). The top 2 biological process terms identified related to cell localization (GO:0051649) and interspecies interaction between organisms (GO:0006810).

The comparison between HDC and UC-MSC EVs yielded 2 clusters with cluster 1 (score=17.6) containing 31 nodes and 264 interaction pairs (FIG. 52). The top 5 hub genes within cluster 1 were related to copper binding (CP, degree=22), iron biding transport (TF, degree=22), thyroid hormone-binding (TTR, degree=22), and vitamin D transport (GC, degree=22). The top biological process terms within cluster 1 were implicated in exocytosis (GO:0045055), platelet degranulation (GO:0002576) and vesicle transport (GO:0016192). Cluster 2 contained 30 nodes and 169 interaction pairs. The top hub genes within this cluster were related to collagen (COL1A1, degree=8; COL1A2, degree=17), glycoprotein (THBS1, degree=17), and matrix metallopeptidases (MMP2, degree=19; MMP14, degree=16). String enrichment analysis within this clusters showed that proteins mediated cell adhesion (GO:0007155), extracellular matrix organization (GO:0030198), and formation of the primary germ layer (GO:0001704).

TABLE 3 shows a list of exemplary differentially expressed miRNA cargo in HDC vs BM-MSC EVs. hsa, Homo sapiens

| Differentially | | Log2 | |
|---|---|---|---|
| Expressed | | Fold | |
| miRNA | Description | Change | p-Value |
| hsa-miR-1224-5p | MI0003764 | 3.51187 | 0.026825 |
| hsa-miR-196a-5p | MI0000279 | 2.53976 | 0.005498 |
| hsa-miR-630 | MI0003644 | 2.30881 | 0.005956 |
| hsa-miR-3140-5p | MI0014163 | 2.14175 | 0.000502 |
| hsa-miR-874-5p | MI0005532 | 2.11602 | 0.049925 |
| hsa-miR-1306-5p | MI0006443 | 2.07554 | 0.015506 |
| hsa-miR-3131 | MI0014151 | 2.04051 | 0.035703 |
| hsa-miR-485-5p | MI0002469 | 2.00733 | 0.00846 |
| hsa-miR-876-3p | MI0005542 | 1.94171 | 0.023983 |
| hsa-miR-3196 | MI0014241 | 1.84552 | 0.046151 |
| hsa-miR-873-3p | MI0005564 | 1.81709 | 0.040183 |
| hsa-miR-4741 | MI0017379 | 1.77139 | 0.006722 |
| hsa-miR-219a-2-3p | MI0000740 | 1.73822 | 0.044009 |
| hsa-miR-34a-5p | MI0000268 | 1.73227 | 0.023769 |
| hsa-miR-4787-3p | MI0017434 | 1.70069 | 0.028831 |
| hsa-miR-520d-3p | MI0003164 | 1.674 | 0.005441 |
| hsa-miR-501-3p | MI0003185 | 1.65044 | 0.04604 |
| hsa-miR-590-3p | MI0003602 | 1.64296 | 0.047512 |
| hsa-miR-10b-5p | MI0000267 | 1.53635 | 0.025041 |
| hsa-miR-578 | MI0003585 | 1.43806 | 0.038703 |
| hsa-miR-519e-3p | MI0003145 | 1.43618 | 0.046268 |
| hsa-miR-5196-5p | MI0018175 | 1.41843 | 0.030021 |
| hsa-miR-199a-5p | MI0000281 | 1.41083 | 0.0096 |
| hsa-miR-3690 | MI0016091 | 1.37058 | 0.026933 |
| hsa-miR-517a-3p | MI0003161 | 1.3701 | 0.035065 |
| hsa-miR-619-3p | MI0003633 | 1.35175 | 0.034291 |
| hsa-miR-568 | MI0003574 | 1.27142 | 0.041672 |
| hsa-miR-625-5p | MI0003639 | 1.07369 | 0.031 |
| hsa-miR-4451 | MI0016797 | 1.04325 | 0.037141 |
| hsa-miR-1180-3p | MI0006273 | 1.04234 | 0.03193 |
| hsa-miR-5010-3p | MI0017878 | 0.997418 | 0.022214 |
| hsa-miR-302e | MI0006417 | 0.979929 | 0.046686 |
| hsa-miR-1258 | MI0006392 | 0.971695 | 0.026819 |
| hsa-miR-197-5p | MI0000239 | 0.967794 | 0.035038 |
| hsa-miR-92a-1-5p | MI0000093 | 0.900024 | 0.029612 |
| hsa-miR-10a-5p | MI0000266 | 0.722411 | 0.031681 |
| hsa-miR-16-5p | MI0000070 | −0.634 | 0.003992 |
| hsa-let-7g-5p | MI0000433 | −0.646361 | 0.03895 |
| hsa-miR-4286 | MI0015894 | −0.707782 | 0.008563 |
| hsa-miR-199b-5p | MI0000282 | −0.823786 | 0.025643 |
| hsa-miR-19b-3p | MI0000074 | −0.86821 | 0.043358 |
| hsa-miR-24-3p | MI0000081 | −0.893848 | 0.002479 |
| hsa-miR-23b-3p | MI0000439 | −0.999376 | 0.001002 |
| hsa-miR-21-5p | MI0000077 | −1.03304 | 0.035481 |
| hsa-miR-376a-3p | MI0003529 | −1.06254 | 0.042989 |
| hsa-miR-337-5p | MI0000806 | −1.19023 | 0.011657 |
| hsa-miR-23a-3p | MI0000079 | −1.2838 | 0.023105 |
| hsa-miR-27b-3p | MI0000440 | −1.31904 | 0.005334 |
| hsa-let-7i-5p | MI0000434 | −1.37666 | 0.002865 |
| hsa-miR-4516 | MI0016882 | −1.41662 | 0.007115 |
| hsa-miR-374a-5p | MI0000782 | −1.44434 | 0.006919 |
| hsa-miR-98-5p | MI0000100 | −1.46162 | 0.013817 |
| hsa-miR-22-3p | MI0000078 | −1.59936 | 0.011553 |
| hsa-miR-29a-3p | MI0000087 | −1.63008 | 0.02321 |
| hsa-miR-4454 + | MI0016800 + | −1.63058 | 0.005845 |
| hsa-miR-7975 | MI0025751 | | |
| hsa-miR-29b-3p | MI0000107 | −3.46125 | 0.036546 |

TABLE 4 shows a list of exemplary differentially expressed miRNA cargo in BM-MSC vs UC-MSC EVs. hsa, Homo sapiens

| Differentially | | Log | |
|---|---|---|---|
| Expressed | | Fold | |
| miRNA | Description | Change | p-Value |
| hsa-miR-765 | MI0005116 | 4.78479 | 0.000392 |
| hsa-miR-223-3p | MI0000300 | 4.57158 | 0.005398 |
| hsa-miR-1234-3p | MI0006324 | 3.39719 | 0.042276 |
| hsa-miR-216a-5p | MI0000292 | 3.38365 | 0.001516 |
| hsa-miR-3605-5p | MI0015995 | 3.34906 | 0.010328 |
| hsa-miR-509-3-5p | MI0005717 | 3.17894 | 0.018589 |
| hsa-miR-147a | MI0000262 | 3.08841 | 0.00572 |
| hsa-miR-516a-5p | MI0003180 | 3.04302 | 0.02371 |
| hsa-miR-501-3p | MI0003185 | 3.01713 | 0.005525 |
| hsa-miR-562 | MI0003568 | 2.97404 | 0.028448 |
| hsa-miR-4421 | MI0016758 | 2.97049 | 0.010323 |
| hsa-miR-4485-3p | MI0016846 | 2.91954 | 0.011102 |
| hsa-miR-346 | MI0000826 | 2.89487 | 0.012048 |
| hsa-miR-802 | MI0003906 | 2.88988 | 0.009402 |
| hsa-miR-1972 | MI0009982 | 2.83784 | 0.017128 |
| hsa-miR-2116-5p | MI0010635 | 2.8182 | 0.005957 |
| hsa-miR-192-5p | MI0000234 | 2.76156 | 0.015987 |
| hsa-miR-448 | MI0001637 | 2.73053 | 0.037179 |
| hsa-miR-626 | MI0003640 | 2.68868 | 0.002277 |
| hsa-mir-217 | MI0000293 | 2.6292 | 0.026176 |
| hsa-miR-1268b | MI0016748 | 2.60507 | 0.022066 |
| hsa-miR-410-3p | MI0002465 | 2.60436 | 0.023953 |
| hsa-miR-2113 | MI0003939 | 2.56856 | 0.043628 |
| hsa-miR-3605-3p | MI0015995 | 2.46141 | 0.023668 |
| hsa-miR-2053 | MI0010487 | 2.44786 | 0.037586 |
| hsa-miR-2110 | MI0010629 | 2.44786 | 0.005049 |
| hsa-mir-1973 | MI0009983 | 2.44178 | 0.010847 |
| hsa-miR-181c-5p | MI0000271 | 2.4404 | 0.04721 |
| hsa-miR-499b-3p | MI0017396 | 2.42823 | 0.041507 |
| hsa-miR-1266-5p | MI0006403 | 2.42226 | 0.023156 |
| hsa-miR-604 | MI0003617 | 2.41311 | 0.022326 |
| hsa-miR-142-3p | MI0000458 | 2.41105 | 0.018546 |
| hsa-miR-298 | MI0005523 | 2.39594 | 0.042096 |
| hsa-miR-3144-3p | MI0014169 | 2.39228 | 0.040791 |
| hsa-miR-224-5p | MI0000301 | 2.38728 | 0.025314 |
| hsa-miR-329-3p | MI0001725 | 2.34055 | 0.010058 |
| hsa-miR-3127-5p | MI0014144 | 2.32543 | 0.042046 |
| hsa-miR-664a-3p | MI0006442 | 2.31971 | 0.01485 |
| hsa-miR-509-3p | MI0005530 | 2.28988 | 0.013687 |
| hsa-miR-1204 | MI0006337 | 2.28391 | 0.018704 |
| hsa-miR-135b-5p | MI0000810 | 2.27723 | 0.040895 |
| hsa-miR-573 | MI0003580 | 2.26642 | 0.048338 |
| hsa-miR-587 | MI0003595 | 2.2624 | 0.023721 |
| hsa-miR-564 | MI0003570 | 2.25139 | 0.030939 |
| hsa-miR-548q | MI0010637 | 2.24804 | 0.009684 |
| hsa-miR-3614-5p | MI0016004 | 2.24737 | 0.031794 |
| hsa-miR-511-5p | MI0003127 | 2.21575 | 0.020545 |
| hsa-miR-335-5p | MI0000816 | 2.20534 | 0.010573 |
| hsa-miR-548e-3p | MI0006344 | 2.20221 | 0.00936 |
| hsa-miR-593-3p | MI0003605 | 2.1812 | 0.020538 |
| hsa-miR-28-5p | MI0000086 | 2.17127 | 0.029427 |
| hsa-miR-552-3p | MI0003557 | 2.15637 | 0.033478 |
| hsa-miR-492 | MI0003131 | 2.14556 | 0.048469 |
| hsa-miR-140-3p | MI0000456 | 2.12807 | 0.009546 |
| hsa-miR-216b-5p | MI0005569 | 2.12807 | 0.010235 |
| hsa-miR-519b-3p | MI0003151 | 2.09704 | 0.035532 |
| hsa-miR-200c-3p | MI0000650 | 2.0949 | 0.009539 |
| hsa-miR-620 | MI0003634 | 2.0942 | 0.038209 |
| hsa-miR-1285-5p | MI0006346 | 2.08623 | 0.030867 |
| hsa-miR-649 | MI0003664 | 2.06386 | 0.021806 |
| hsa-miR-503-3p | MI0003188 | 2.0404 | 0.01737 |
| hsa-miR-3150b-3p | MI0016426 | 2.03068 | 0.027953 |
| hsa-miR-761 | MI0003941 | 2.01233 | 0.013307 |
| hsa-miR-512-5p | MI0003140 | 2.00951 | 0.034654 |
| hsa-miR-936 | MI0005758 | 2.00722 | 0.010913 |
| hsa-miR-378d | MI0016749 | 1.98973 | 0.035253 |
| hsa-miR-1287-5p | MI0006349 | 1.96873 | 0.012094 |
| hsa-miR-3131 | MI0014151 | 1.95655 | 0.045858 |
| hsa-miR-514a-3p | MI0003198 | 1.95655 | 0.003203 |
| hsa-miR-211-5p | MI0000287 | 1.95566 | 0.035346 |
| hsa-miR-542-3p | MI0003686 | 1.95234 | 0.009111 |
| hsa-miR-1254 | MIMAT0005905 | 1.94663 | 0.003349 |
| hsa-miR-433-3p | MI0001723 | 1.93703 | 0.02841 |
| hsa-miR-1827 | MI0008195 | 1.92974 | 0.025837 |
| hsa-miR-146a-5p | MI0000477 | 1.92773 | 0.035806 |
| hsa-miR-182-5p | MI0000272 | 1.92551 | 0.010956 |
| hsa-miR-548ak | MI0016840 | 1.91954 | 0.042506 |
| hsa-miR-193a-3p | MI0000487 | 1.91169 | 0.045262 |
| hsa-miR-548g-3p | MI0006395 | 1.91072 | 0.016864 |
| hsa-miR-150-5p | MI0000479 | 1.90337 | 0.041059 |
| hsa-miR-507 | MI0003194 | 1.89991 | 0.031563 |
| hsa-miR-575 | MI0003582 | 1.87508 | 0.032462 |
| hsa-miR-1272 | MI0006408 | 1.86887 | 0.039317 |
| hsa-miR-1537-3p | MI0007258 | 1.85001 | 0.025031 |
| hsa-miR-378b | MI0014154 | 1.84924 | 0.030926 |
| hsa-miR-126-3p | MI0000471 | 1.84736 | 0.03741 |
| hsa-miR-526a + | MI0003157 + | 1.84541 | 0.033821 |
| hsa-miR-518c-5p + | MI0003159 + | | |
| hsa-miR-518d-5p | MIMAT0005456 | | |
| hsa-miR-96-5p | MI0000098 | 1.84541 | 0.020691 |
| hsa-miR-431-5p | MI0001721 | 1.83658 | 0.024391 |
| hsa-miR-1279 | MI0006426 | 1.83038 | 0.035459 |
| hsa-miR-4461 | MI0016807 | 1.83038 | 0.049484 |
| hsa-miR-885-3p | MI0005560 | 1.81223 | 0.032137 |
| hsa-miR-548v | MI0014174 | 1.81192 | 0.048668 |
| hsa-miR-5010-3p | MI0017878 | 1.80954 | 0.021485 |
| hsa-miR-1245b-3p | MI0017431 | 1.78503 | 0.00655 |
| hsa-miR-3180-3p | MI0014214 | 1.78119 | 0.032067 |
| hsa-miR-510-3p | MI0003197 | 1.77374 | 0.018022 |
| hsa-miR-520c-3p | MI0003158 | 1.75882 | 0.039709 |
| hsa-miR-585-3p | MI0003592 | 1.75528 | 0.002864 |
| hsa-miR-486-3p | MI0002470 | 1.73053 | 0.01817 |
| hsa-miR-548d-5p | MI0003671 | 1.73053 | 0.04677 |
| hsa-miR-1276 | MI0006416 | 1.72928 | 0.046612 |
| hsa-miR-548d-3p | MI0003671 | 1.72061 | 0.028482 |
| hsa-miR-1264 | MI0003758 | 1.71698 | 0.015075 |
| hsa-miR-148b-3p | MI0000811 | 1.69352 | 0.000543 |
| hsa-miR-1224-3p | MI0003764 | 1.68606 | 0.024003 |
| hsa-miR-302c-3p | MI0000773 | 1.67872 | 0.021374 |
| hsa-miR-1269b | MI0016888 | 1.66506 | 0.04456 |
| hsa-miR-605-5p | MI0003618 | 1.66288 | 0.049825 |
| hsa-miR-1206 | MI0006339 | 1.65909 | 0.016673 |
| hsa-miR-1307-5p | MI0006444 | 1.65042 | 0.036926 |
| hsa-miR-1297 | MI0006358 | 1.65026 | 0.025853 |
| hsa-miR-608 | MI0003621 | 1.63293 | 0.007805 |
| hsa-miR-631 | MI0003645 | 1.63204 | 0.035562 |
| hsa-miR-378f | MI0016756 | 1.62322 | 0.010012 |
| hsa-miR-582-3p | MI0003589 | 1.62184 | 0.026641 |
| hsa-miR-139-3p | MI0000261 | 1.61193 | 0.016123 |
| hsa-miR-145-5p | MI0000461 | 1.61054 | 0.016788 |
| hsa-miR-5001-5p | MI0017867 | 1.60573 | 0.046191 |
| hsa-miR-561-5p | MI0003567 | 1.59975 | 0.034076 |
| hsa-miR-421 | MI0003685 | 1.58621 | 0.017424 |
| hsa-miR-500a-5p + | MIMAT0004773 + | 1.58621 | 0.047978 |
| hsa-miR-501-5p | MI0003185 | | |
| hsa-miR-1257 | MI0006391 | 1.58335 | 0.048896 |
| hsa-miR-924 | MI0005716 | 1.56657 | 0.049312 |
| hsa-miR-1323 | MI0003786 | 1.55914 | 0.030934 |
| hsa-miR-30e-3p | MI0000749 | 1.52703 | 0.021909 |
| hsa-miR-520d-3p | MI0003164 | 1.52562 | 0.026794 |
| hsa-miR-3130-3p | MI0014147 | 1.51591 | 0.008186 |
| hsa-miR-6720-3p | MI0022555 | 1.49306 | 0.045228 |
| hsa-miR-520a-5p | MI0003149 | 1.47604 | 0.030135 |
| hsa-miR-92a-1-5p | MI0000093 | 1.46141 | 0.002827 |
| hsa-mir-520e | MI0003143 | 1.42823 | 0.037383 |
| hsa-miR-4787-5p | MI0017434 | 1.42226 | 0.031137 |
| hsa-miR-548h-5p | MI0006411 | 1.42226 | 0.0192 |
| hsa-miR-193b-3p | MI0003137 | 1.41557 | 0.03078 |
| hsa-miR-302b-3p | MI0000772 | 1.41468 | 0.042956 |
| hsa-miR-551b-3p | MI0003575 | 1.3859 | 0.019962 |
| hsa-miR-106b-5p | MI0000734 | 1.36752 | 0.013117 |
| hsa-miR-363-5p | MI0000764 | 1.36401 | 0.016555 |
| hsa-miR-1180-3p | MI0006273 | 1.32881 | 0.02409 |
| hsa-miR-337-5p | MI0000806 | 1.32575 | 0.000854 |
| hsa-miR-30a-3p | MI0000088 | 1.30737 | 0.024367 |
| hsa-miR-502-3p | MI0003186 | 1.30737 | 0.033273 |
| hsa-miR-30d-5p | MI0000255 | 1.3005 | 0.015316 |
| hsa-miR-98-5p | MI0000100 | 1.30003 | 0.026804 |
| hsa-miR-548n | MI0006399 | 1.29214 | 0.024043 |
| hsa-miR-597-5p | MI0003609 | 1.27969 | 0.035604 |
| hsa-miR-671-3p | MI0003760 | 1.26642 | 0.046731 |
| hsa-miR-769-3p | MI0003834 | 1.26642 | 0.046955 |
| hsa-miR-584-3p | MI0003591 | 1.25979 | 0.042083 |
| hsa-miR-1250-5p | MI0006385 | 1.22727 | 0.02185 |
| hsa-miR-211-3p | MI0000287 | 1.15911 | 0.001896 |
| hsa-miR-4454 + | MI0016800 + | 1.12424 | 0.045843 |
| hsa-miR-7975 | MI0025751 | | |
| hsa-miR-548e-5p | MI0006344 | 1.1057 | 0.037154 |
| hsa-miR-4286 | MI0015894 | 1.1 | 0.012917 |
| hsa-miR-377-3p | MI0000785 | 1.04337 | 0.005261 |
| hsa-miR-432-5p | MI0003133 | 0.976695 | 0.043256 |
| hsa-miR-526b-5p | MI0003150 | 0.960574 | 0.029791 |
| hsa-miR-543 | MI0005565 | 0.954208 | 0.041838 |
| hsa-miR-337-3p | MI0000806 | 0.953233 | 0.036074 |
| hsa-miR-487b-3p | MI0003530 | 0.950576 | 0.024347 |
| hsa-miR-342-3p | MI0000805 | 0.890345 | 0.040172 |
| hsa-miR-423-3p | MI0001445 | 0.867621 | 0.015293 |
| hsa-miR-19a-3p | MI0000073 | 0.722647 | 0.001341 |
| hsa-miR-199b-5p | MI0000282 | −0.795716 | 0.040583 |
| hsa-let-7d-5p | MI0000065 | −0.826208 | 0.047614 |
| hsa-miR-630 | MI0003644 | −0.883867 | 0.04198 |
| hsa-miR-199a-5p | MI0000281 | −1.50399 | 0.007089 |
| hsa-miR-34a-5p | MI0000268 | −1.53395 | 0.012176 |
| hsa-miR-93-5p | MI0000095 | −1.65321 | 0.042628 |
| hsa-miR-15a-5p | MI0000069 | −1.66791 | 0.046804 |
| hsa-miR-181a-5p | MI0000289 | −2.06005 | 0.017154 |
| hsa-miR-1915-3p | MI0008336 | −2.10681 | 0.007254 |
| hsa-miR-199a-3p + | MI0000242 + | −2.29495 | 0.023153 |
| hsa-miR-199b-3p | MI0000282 | | |
| hsa-let-7b-5p | MI0000063 | −2.8971 | 0.007389 |
| hsa-let-7a-5p | MI0000061 | −5.76857 | 0.031576 |

TABLE 5 shows a list of exemplary differentially expressed miRNA cargo in HDC vs UC-MSC EVs. hsa}, Homo sapiens

| Differentially | | Log | |
|---|---|---|---|
| Expressed | | Fold | |
| miRNA | Description | Change | p-Value |
| hsa-miR-223-3p | MI0000300 | 5.33029 | 0.008513 |
| hsa-miR-765 | MI0005116 | 4.93721 | 0.002055 |
| hsa-miR-1234-3p | MI0006324 | 4.13177 | 0.01968 |
| hsa-miR-501-3p | MI0003185 | 3.98999 | 0.005476 |
| hsa-miR-3131 | MI0014151 | 3.88502 | 0.011313 |
| hsa-miR-216a-5p | MI0000292 | 3.87323 | 0.001157 |
| hsa-miR-516a-5p | MI0003180 | 3.80753 | 0.010409 |
| hsa-miR-573 | MI0003580 | 3.77656 | 0.011032 |
| hsa-miR-509-3-5p | MI0005717 | 3.72402 | 0.013596 |
| hsa-miR-147a | MI0000262 | 3.70877 | 0.004537 |
| hsa-miR-329-3p | MI0001725 | 3.70056 | 0.019194 |
| hsa-miR-1972 | MI0009982 | 3.6267 | 0.032341 |
| hsa-mir-217 | MI0000293 | 3.58859 | 0.010542 |
| hsa-miR-591 | MI0003603 | 3.56235 | 0.021699 |
| hsa-miR-4421 | MI0016758 | 3.54724 | 0.004063 |
| hsa-miR-1269a | MI0006406 | 3.52065 | 0.016652 |
| hsa-miR-499b-3p | MI0017396 | 3.4968 | 0.005787 |
| hsa-miR-503-3p | MI0003188 | 3.3984 | 0.002617 |
| hsa-miR-298 | MI0005523 | 3.35846 | 0.000462 |
| hsa-miR-548e-3p | MI0006344 | 3.32509 | 0.013531 |
| hsa-miR-4485-3p | MI0016846 | 3.31387 | 0.005365 |
| hsa-miR-587 | MI0003595 | 3.31172 | 0.019121 |
| hsa-miR-513c-5p | MI0006649 | 3.30005 | 0.03381 |
| hsa-miR-520d-3p | MI0003164 | 3.27078 | 0.003883 |
| hsa-miR-885-3p | MI0005560 | 3.26576 | 0.024047 |
| hsa-miR-224-5p | MI0000301 | 3.26321 | 0.009714 |
| hsa-miR-1285-3p | MI0006347 | 3.26307 | 0.028924 |
| hsa-miR-562 | MI0003568 | 3.24939 | 0.016325 |
| hsa-miR-4448 | MI0016791 | 3.20656 | 0.002942 |
| hsa-miR-346 | MI0000826 | 3.20028 | 0.004135 |
| hsa-miR-3605-3p | MI0015995 | 3.19489 | 0.011957 |
| hsa-miR-2113 | MI0003939 | 3.18908 | 0.024191 |
| hsa-miR-1249-5p | MI0006384 | 3.16385 | 0.017464 |
| hsa-miR-378d | MI0016749 | 3.14324 | 0.001049 |
| hsa-miR-761 | MI0003941 | 3.13243 | 0.002336 |
| hsa-miR-3614-5p | MI0016004 | 3.12841 | 0.002975 |
| hsa-miR-200c-3p | MI0000650 | 3.09926 | 0.001846 |
| hsa-miR-1268b | MI0016748 | 3.09465 | 0.024365 |
| hsa-miR-631 | MI0003645 | 3.09059 | 0.009538 |
| hsa-miR-139-3p | MI0000261 | 3.08758 | 0.005165 |
| hsa-miR-192-5p | MI0000234 | 3.07579 | 0.014225 |
| hsa-miR-448 | MI0001637 | 3.06362 | 0.032386 |
| hsa-miR-509-3p | MI0005530 | 3.0583 | 0.010062 |
| hsa-miR-196a-5p | MI0000279 | 3.04896 | 0.002125 |
| hsa-miR-548ak | MI0016840 | 3.04476 | 0.001126 |
| hsa-miR-620 | MI0003634 | 3.00291 | 0.000897 |
| hsa-miR-2110 | MI0010629 | 2.96403 | 0.047704 |
| hsa-miR-595 | MI0003607 | 2.96091 | 0.034747 |
| hsa-miR-624-3p | MI0003638 | 2.95708 | 0.04766 |
| hsa-miR-4741 | MI0017379 | 2.93745 | 0.009705 |
| hsa-miR-568 | MI0003574 | 2.93745 | 0.048598 |
| hsa-miR-1254 | MIMAT0005905 | 2.9091 | 0.011401 |
| hsa-miR-4425 | MI0016764 | 2.90641 | 0.039821 |
| hsa-miR-181c-5p | MI0000271 | 2.90427 | 0.02129 |
| hsa-miR-593-3p | MI0003605 | 2.90427 | 0.009797 |
| hsa-miR-5010-3p | MI0017878 | 2.87811 | 0.005943 |
| hsa-miR-496 | MI0003136 | 2.87323 | 0.034217 |
| hsa-miR-3196 | MI0014241 | 2.8583 | 0.004262 |
| hsa-miR-548al | MI0016851 | 2.85616 | 0.006636 |
| hsa-miR-548b-3p | MI0003596 | 2.82142 | 0.032678 |
| hsa-miR-802 | MI0003906 | 2.81796 | 0.005801 |
| hsa-miR-655-3p | MI0003677 | 2.80792 | 0.03402 |
| hsa-miR-1226-3p | MI0006313 | 2.79801 | 0.005215 |
| hsa-miR-1279 | MI0006426 | 2.7843 | 0.005141 |
| hsa-miR-1258 | MI0006392 | 2.77584 | 0.02787 |
| hsa-miR-1266-5p | MI0006403 | 2.77349 | 0.016058 |
| hsa-miR-548h-5p | MI0006411 | 2.77062 | 0.004467 |
| hsa-miR-96-5p | MI0000098 | 2.76209 | 0.010065 |
| hsa-miR-491-5p | MI0003126 | 2.76022 | 0.020797 |
| hsa-miR-649 | MI0003664 | 2.75601 | 0.009121 |
| hsa-miR-150-5p | MI0000479 | 2.74991 | 0.000219 |
| hsa-miR-3130-3p | MI0014147 | 2.73174 | 0.001416 |
| hsa-miR-2116-5p | MI0010635 | 2.7288 | 0.008441 |
| hsa-miR-1287-5p | MI0006349 | 2.72497 | 0.003194 |
| hsa-miR-215-5p | MI0000291 | 2.72321 | 0.018357 |
| hsa-miR-5480-3p + | MI0006402 + | 2.72009 | 0.025234 |
| hsa-miR-548ah-3p + | MI0016796 + | | |
| hsa-miR-548av-3p | MI0019152 | | |
| hsa-miR-483-5p | MI0002467 | 2.71142 | 0.027086 |
| hsa-miR-526a + | MI0003157 + | 2.71142 | 0.012786 |
| hsa-miR-518c-5p + | MI0003159 + | | |
| hsa-miR-518d-5p | MI0003171 | | |
| hsa-miR-744-5p | MI0005559 | 2.71142 | 0.026766 |
| hsa-miR-626 | MI0003640 | 2.70061 | 0.002461 |
| hsa-miR-140-3p | MI0000456 | 2.69179 | 0.039797 |
| hsa-miR-517a-3p | MI0003161 | 2.69179 | 0.007914 |
| hsa-miR-95-3p | MI0000097 | 2.69179 | 0.022125 |
| hsa-miR-1252-5p | MI0006434 | 2.6916 | 0.008623 |
| hsa-miR-1224-3p | MI0003764 | 2.69064 | 0.043516 |
| hsa-miR-1264 | MI0003758 | 2.67824 | 0.003006 |
| hsa-miR-507 | MI0003194 | 2.66833 | 0.008166 |
| hsa-miR-200b-3p | MI0000342 | 2.66075 | 0.049683 |
| hsa-miR-876-3p | MI0005542 | 2.66075 | 0.017757 |
| hsa-miR-640 | MI0003655 | 2.65966 | 0.041804 |
| hsa-miR-556-3p | MI0003562 | 2.65861 | 0.042162 |
| hsa-miR-9-5p | MI0000466 | 2.65574 | 0.0112 |
| hsa-miR-520a-5p | MI0003149 | 2.64721 | 0.022974 |
| hsa-miR-576-3p | MI0003583 | 2.64261 | 0.04434 |
| hsa-miR-105-5p | MI0000111 | 2.63729 | 0.033763 |
| hsa-miR-197-3p | MI0000239 | 2.63729 | 0.001336 |
| hsa-miR-330-5p | MI0000803 | 2.63729 | 0.00842 |
| hsa-miR-492 | MI0003131 | 2.63515 | 0.028966 |
| hsa-miR-342-5p | MI0000805 | 2.62758 | 0.001828 |
| hsa-miR-3690 | MI0016091 | 2.62226 | 0.015028 |
| hsa-miR-216b-5p | MI0005569 | 2.61891 | 0.004538 |
| hsa-miR-1269b | MI0016888 | 2.6068 | 0.016969 |
| hsa-miR-5196-5p | MI0018175 | 2.60411 | 0.035382 |
| hsa-miR-548d-3p | MI0003671 | 2.60411 | 0.008187 |
| hsa-miR-1257 | MI0006391 | 2.59057 | 0.013654 |
| hsa-miR-2053 | MI0010487 | 2.59057 | 0.035164 |
| hsa-miR-3182 | MI0014224 | 2.59057 | 0.021346 |
| hsa-miR-502-3p | MI0003186 | 2.58272 | 0.004202 |
| hsa-miR-512-5p | MI0003140 | 2.58174 | 0.022984 |
| hsa-miR-142-3p | MI0000458 | 2.57976 | 0.007233 |
| hsa-miR-506-3p | MI0003193 | 2.5671 | 0.007563 |
| hsa-miR-1469 | MI0007074 | 2.56221 | 0.027298 |
| hsa-miR-652-3p | MI0003667 | 2.56194 | 0.024254 |
| hsa-miR-760 | MI0005567 | 2.55926 | 0.033154 |
| hsa-miR-1178-3p | MI0006271 | 2.55345 | 0.004484 |
| hsa-miR-302e | MI0006417 | 2.52998 | 0.031092 |
| hsa-miR-1271-5p | MI0003814 | 2.52822 | 0.019451 |
| hsa-miR-92a-3p | MI0000093 | 2.51781 | 0.005419 |
| hsa-miR-211-5p | MI0000287 | 2.51554 | 0.024137 |
| hsa-miR-212-3p | MI0000288 | 2.50495 | 0.004815 |
| hsa-miR-1233-3p | MI0006323 | 2.50476 | 0.043054 |
| hsa-miR-567 | MI0003573 | 2.4999 | 0.049065 |
| hsa-miR-6720-3p | MI0022555 | 2.49895 | 0.025004 |
| hsa-miR-770-5p | MI0005118 | 2.48808 | 0.020254 |
| hsa-miR-3150b-3p | MI0016426 | 2.48594 | 0.018919 |
| hsa-miR-519b-3p | MI0003151 | 2.46577 | 0.010332 |
| hsa-miR-940 | MI0005762 | 2.45222 | 0.00779 |
| hsa-miR-323b-3p | MI0014206 | 2.44544 | 0.037025 |
| hsa-miR-190a-5p | MI0000486 | 2.4425 | 0.027064 |
| hsa-miR-1180-3p | MI0006273 | 2.4423 | 0.006414 |
| hsa-miR-1204 | MI0006337 | 2.4423 | 0.014778 |
| hsa-miR-576-5p | MI0003583 | 2.4423 | 0.010629 |
| hsa-miR-92a-1-5p | MI0000093 | 2.43259 | 0.000958 |
| hsa-miR-924 | MI0005716 | 2.42944 | 0.008023 |
| hsa-miR-500a-5p + | MI0003184 + | 2.42091 | 0.044677 |
| hsa-miR-501-5p | MI0003185 | | |
| hsa-miR-1306-5p | MI0006443 | 2.40913 | 0.013391 |
| hsa-miR-410-3p | MI0002465 | 2.40913 | 0.019 |
| hsa-miR-605-5p | MI0003618 | 2.40913 | 0.017985 |
| hsa-miR-193a-3p | MI0000487 | 2.39624 | 0.030297 |
| hsa-miR-3934-5p | MI0016590 | 2.38949 | 0.03944 |
| hsa-miR-604 | MI0003617 | 2.38077 | 0.016837 |
| hsa-miR-520h | MI0003175 | 2.34491 | 0.004479 |
| hsa-miR-671-3p | MI0003760 | 2.34491 | 0.00034 |
| hsa-miR-1236-3p | MI0006326 | 2.33499 | 0.027455 |
| hsa-miR-3180-3p | MI0014214 | 2.32145 | 0.015817 |
| hsa-miR-518c-3p | MI0003159 | 2.32145 | 0.008111 |
| hsa-miR-452-5p | MI0001733 | 2.31656 | 0.015839 |
| hsa-miR-580-3p | MI0003587 | 2.31249 | 0.019495 |
| hsa-miR-1255b-5p | MI0006436 | 2.29041 | 0.044874 |
| hsa-miR-1301-3p | MI0003815 | 2.28827 | 0.010722 |
| hsa-miR-182-5p | MI0000272 | 2.28256 | 0.001441 |
| hsa-miR-3185 | MI0014227 | 2.28256 | 0.048772 |
| hsa-miR-1245b-3p | MI0017431 | 2.27078 | 0.004035 |
| hsa-miR-378b | MI0014154 | 2.27078 | 0.010571 |
| hsa-miR-488-3p | MI0003123 | 2.26321 | 0.027281 |
| hsa-miR-1304-5p | MI0006371 | 2.2591 | 0.023802 |
| hsa-miR-1537-3p | MI0007258 | 2.25723 | 0.024512 |
| hsa-miR-1276 | MI0006416 | 2.25115 | 0.018908 |
| hsa-miR-892b | MI0005538 | 2.24732 | 0.020675 |
| hsa-miR-566 | MI0003572 | 2.2376 | 0.022579 |
| hsa-miR-767-5p | MI0003763 | 2.23163 | 0.029228 |
| hsa-miR-542-3p | MI0003686 | 2.23026 | 0.006873 |
| hsa-miR-514a-3p | MI0003198 | 2.23003 | 0.024771 |
| hsa-miR-450b-3p | MI0005531 | 2.22894 | 0.022043 |
| hsa-miR-3605-5p | MI0015995 | 2.22725 | 0.022376 |
| hsa-miR-3144-3p | MI0014169 | 2.22573 | 0.045649 |
| hsa-miR-3074-3p | MI0014181 | 2.22011 | 0.044323 |
| hsa-miR-936 | MI0005758 | 2.21414 | 0.008331 |
| hsa-miR-499a-3p | MI0003183 | 2.20656 | 0.004035 |
| hsa-miR-1285-5p | MI0006346 | 2.18761 | 0.020067 |
| hsa-miR-608 | MI0003621 | 2.18399 | 0.010711 |
| hsa-miR-491-3p | MI0003126 | 2.1831 | 0.02185 |
| hsa-miR-1297 | MI0006358 | 2.17702 | 0.008618 |
| hsa-mir-320a | MI0000542 | 2.17525 | 0.040163 |
| hsa-miR-664a-3p | MI0006442 | 2.17367 | 0.020064 |
| hsa-miR-1206 | MI0006339 | 2.16347 | 0.018777 |
| hsa-miR-548z + | MI0016688 + | 2.16347 | 0.026201 |
| hsa-miR-548h-3p | MI0006414 | | |
| hsa-miR-367-3p | MI0000775 | 2.14992 | 0.029994 |
| hsa-miR-4792 | MI0017439 | 2.14001 | 0.039404 |
| hsa-miR-3918 | MI0016424 | 2.13243 | 0.033661 |
| hsa-miR-489-3p | MI0003124 | 2.13243 | 0.024045 |
| hsa-miR-1224-5p | MI0003764 | 2.12742 | 0.021353 |
| hsa-miR-3127-5p | MI0014144 | 2.12646 | 0.034671 |
| hsa-miR-145-5p | MI0000461 | 2.12226 | 0.006141 |
| hsa-miR-4451 | MI0016797 | 2.10683 | 0.046093 |
| hsa-miR-3151-5p | MI0014178 | 2.07579 | 0.024807 |
| hsa-miR-203a-3p | MI0000283 | 2.06795 | 0.046463 |
| hsa-miR-891b | MI0005534 | 2.06713 | 0.01925 |
| hsa-miR-4787-5p | MI0017434 | 2.06099 | 0.013962 |
| hsa-miR-126-3p | MI0000471 | 2.0583 | 0.042081 |
| hsa-miR-494-5p | MI0003134 | 2.04744 | 0.04419 |
| hsa-miR-302b-3p | MI0000772 | 2.04476 | 0.020115 |
| hsa-miR-219a-2-3p | MI0000740 | 2.03093 | 0.031451 |
| hsa-miR-1244 | MI0015975 | 2.02996 | 0.04706 |
| hsa-miR-433-3p | MI0001723 | 2.02996 | 0.027666 |
| hsa-miR-196b-5p | MI0001150 | 2.02512 | 0.014482 |
| hsa-miR-564 | MI0003570 | 2.02512 | 0.042078 |
| hsa-miR-548ah-5p | MI0016796 | 2.02024 | 0.005515 |
| hsa-miR-4461 | MI0016807 | 2.01981 | 0.036169 |
| hsa-miR-513b-5p | MI0006648 | 2.00166 | 0.040061 |
| hsa-miR-1272 | MI0006408 | 1.99195 | 0.003487 |
| hsa-miR-578 | MI0003585 | 1.98811 | 0.015184 |
| hsa-miR-28-5p | MI0000086 | 1.97255 | 0.046737 |
| hsa-miR-890 | MI0005533 | 1.96848 | 0.047678 |
| hsa-miR-510-3p | MI0003197 | 1.95708 | 0.015278 |
| hsa-miR-2117 | MI0010636 | 1.95494 | 0.005515 |
| hsa-miR-1200 | MI0006332 | 1.94962 | 0.028438 |
| hsa-miR-548v | MI0014174 | 1.94596 | 0.043815 |
| hsa-miR-486-3p | MI0002470 | 1.92987 | 0.032294 |
| hsa-miR-892a | MI0005528 | 1.91507 | 0.01711 |
| hsa-miR-561-5p | MI0003567 | 1.90641 | 0.040533 |
| hsa-miR-548g-3p | MI0006395 | 1.90427 | 0.013653 |
| hsa-miR-575 | MI0003582 | 1.89967 | 0.013006 |
| hsa-miR-1270 | MI0006407 | 1.89435 | 0.031811 |
| hsa-miR-4458 | MI0016804 | 1.89435 | 0.00553 |
| hsa-miR-1250-5p | MI0006385 | 1.8808 | 0.015905 |
| hsa-miR-450a-2-3p | MI0003187 | 1.8808 | 0.046709 |
| hsa-miR-432-5p | MI0003133 | 1.85806 | 0.018567 |
| hsa-miR-205-5p | MI0000285 | 1.84977 | 0.013262 |
| hsa-miR-384 | MI0001145 | 1.83014 | 0.028782 |
| hsa-miR-888-5p | MI0005537 | 1.82659 | 0.034249 |
| hsa-miR-4521 | MI0016887 | 1.81659 | 0.016877 |
| hsa-miR-548k | MI0006354 | 1.81659 | 0.039074 |
| hsa-miR-875-3p | MI0005541 | 1.7991 | 0.029107 |
| hsa-miR-520c-3p | MI0003158 | 1.79422 | 0.028392 |
| hsa-miR-146b-3p | MI0003129 | 1.78509 | 0.035265 |
| hsa-miR-1827 | MI0008195 | 1.78095 | 0.037258 |
| hsa-miR-610 | MI0003623 | 1.77947 | 0.010076 |
| hsa-miR-1323 | MI0003786 | 1.77475 | 0.032109 |
| hsa-miR-1295a | MI0006357 | 1.77407 | 0.02679 |
| hsa-miR-34c-3p | MI0000743 | 1.76209 | 0.045473 |
| hsa-miR-431-5p | MI0001721 | 1.76061 | 0.030662 |
| hsa-miR-584-3p | MI0003591 | 1.75217 | 0.01843 |
| hsa-miR-3136-5p | MI0014158 | 1.74246 | 0.036117 |
| hsa-miR-378c | MI0015825 | 1.74246 | 0.015863 |
| hsa-miR-4787-3p | MI0017434 | 1.74246 | 0.023805 |
| hsa-mir-1973 | MI0009983 | 1.73254 | 0.016222 |
| hsa-miR-516b-5p | MI0003167 | 1.72891 | 0.016692 |
| hsa-miR-515-3p | MI0003144 | 1.72283 | 0.035034 |
| hsa-miR-585-3p | MI0003592 | 1.70722 | 0.001712 |
| hsa-miR-3192-5p | MI0014237 | 1.69179 | 0.017086 |
| hsa-mir-378g | MI0016761 | 1.68961 | 0.041401 |
| hsa-mir-375 | MI0000783 | 1.66833 | 0.045878 |
| hsa-miR-4707-3p | MI0017340 | 1.66833 | 0.043869 |
| hsa-miR-582-3p | MI0003589 | 1.65478 | 0.01919 |
| hsa-miR-146a-5p | MI0000477 | 1.64334 | 0.044553 |
| hsa-miR-4755-5p | MI0017395 | 1.63515 | 0.029142 |
| hsa-miR-589-5p | MI0003599 | 1.63515 | 0.031426 |
| hsa-miR-378f | MI0016756 | 1.61197 | 0.009395 |
| hsa-miR-769-3p | MI0003834 | 1.60895 | 0.026116 |
| hsa-miR-1260b | MI0014197 | 1.60411 | 0.023285 |
| hsa-miR-3179 | MI0014213 | 1.60411 | 0.023167 |
| hsa-miR-490-3p | MI0003125 | 1.58065 | 0.025446 |
| hsa-miR-335-5p | MI0000816 | 1.53311 | 0.03145 |
| hsa-miR-193b-3p | MI0003137 | 1.51495 | 0.017177 |
| hsa-miR-363-5p | MI0000764 | 1.51429 | 0.012761 |
| hsa-miR-511-5p | MI0003127 | 1.49895 | 0.02368 |
| hsa-miR-630 | MI0003644 | 1.4961 | 0.02027 |
| hsa-miR-1287-3p | MI0006349 | 1.47334 | 0.047378 |
| hsa-miR-219b-3p | MI0017299 | 1.47334 | 0.031855 |
| hsa-miR-555 | MI0003561 | 1.47334 | 0.035778 |
| hsa-miR-522-3p | MI0003177 | 1.46577 | 0.035875 |
| hsa-miR-1275 | MI0006415 | 1.44614 | 0.037443 |
| hsa-miR-30d-5p | MI0000255 | 1.42727 | 0.008076 |
| hsa-miR-10b-5p | MI0000267 | 1.41974 | 0.047207 |
| hsa-miR-206 | MI0000490 | 1.40913 | 0.015765 |
| hsa-miR-6511a-5p | MI0022223 | 1.40913 | 0.036592 |
| hsa-miR-874-3p | MI0005532 | 1.40913 | 0.045388 |
| hsa-miR-518d-3p | MI0003171 | 1.39921 | 0.024898 |
| hsa-miR-1322 | MI0006653 | 1.3793 | 0.013114 |
| hsa-miR-1260a | MI0006394 | 1.37809 | 0.01651 |
| hsa-miR-148b-3p | MI0000811 | 1.37211 | 0.000826 |
| hsa-miR-889-3p | MI0005540 | 1.37063 | 0.017915 |
| hsa-miR-135a-5p | MI0000453 | 1.36603 | 0.037685 |
| hsa-miR-551b-3p | MI0003575 | 1.36426 | 0.024337 |
| hsa-miR-485-3p | MI0002469 | 1.35248 | 0.04542 |
| hsa-miR-664b-5p | MI0019134 | 1.35248 | 0.015923 |
| hsa-miR-1262 | MI0006397 | 1.3396 | 0.024601 |
| hsa-miR-421 | MI0003685 | 1.31262 | 0.032691 |
| hsa-miR-320d | MI0008190 | 1.30182 | 0.032769 |
| hsa-miR-6724-5p | MI0022559 | 1.30182 | 0.037464 |
| hsa-miR-644a | MI0003659 | 1.27078 | 0.017279 |
| hsa-miR-30e-3p | MI0000749 | 1.26481 | 0.04985 |
| hsa-miR-106b-5p | MI0000734 | 1.26211 | 0.023098 |
| hsa-miR-1299 | MI0006359 | 1.25812 | 0.045838 |
| hsa-miR-887-3p | MI0005562 | 1.24732 | 0.018241 |
| hsa-miR-361-3p | MI0000760 | 1.20882 | 0.046777 |
| hsa-miR-597-5p | MI0003609 | 1.20656 | 0.015173 |
| hsa-miR-651-5p | MI0003666 | 1.14001 | 0.032802 |
| hsa-miR-342-3p | MI0000805 | 1.07716 | 0.024633 |
| hsa-miR-155-5p | MI0000681 | 1.06865 | 0.008355 |
| hsa-miR-30a-3p | MI0000088 | 1.04953 | 0.049902 |
| hsa-miR-376c-5p | MI0000776 | 1.02129 | 0.033543 |
| hsa-miR-4531 | MI0016898 | 0.970625 | 0.045213 |
| hsa-miR-1908-5p | MI0008329 | 0.968483 | 0.006126 |
| hsa-miR-374c-5p | MI0016684 | 0.968483 | 0.007468 |
| hsa-miR-141-3p | MI0000457 | 0.937446 | 0.004174 |
| hsa-miR-487b-3p | MI0003530 | 0.720087 | 0.034532 |
| hsa-let-7g-5p | MI0000433 | −0.76136 | 0.010099 |
| hsa-let-7d-5p | MI0000065 | −1.28254 | 0.011394 |
| hsa-miR-140-5p | MI0000456 | −1.38831 | 0.034678 |
| hsa-let-7i-5p | MI0000434 | −1.48268 | 0.00418 |
| hsa-miR-199b-5p | MI0000282 | −1.54835 | 0.004609 |
| hsa-miR-181a-5p | MI0000289 | −1.60508 | 0.043778 |
| hsa-miR-93-5p | MI0000095 | −1.70293 | 0.040842 |
| hsa-miR-22-3p | MI0000078 | −1.81403 | 0.025015 |
| hsa-miR-21-5p | MI0000077 | −1.82515 | 0.011066 |
| hsa-miR-24-3p | MI0000081 | −1.86573 | 0.024126 |
| hsa-miR-15a-5p | MI0000069 | −2.15744 | 0.016223 |
| hsa-miR-199a-3p + | MI0000242 + | −2.40735 | 0.017088 |
| hsa-miR-199b-3p | MI0000282 | | |
| hsa-miR-29b-3p | MI0000107 | −2.9007 | 0.000435 |
| hsa-let-7b-5p | MI0000063 | −3.2038 | 1.89E−05 |
| hsa-let-7a-5p | MI0000061 | −6.35295 | 0.034528 |

TABLE 6 shows a list of exemplary differentially expressed protein cargo in HDC vs BM-MSC EVs

| | | Log2 | |
|---|---|---|---|
| Protein | Gene | Difference | p value |
| Ectonucleotide | ENPP1 | 5.113114039 | 0.001777813 |
| pyrophosphatase/phosphodiesterase family | | | |
| member 1; Alkaline phosphodiesterase I; | | | |
| Nucleotide pyrophosphatase | | | |
| Caldesmon | CALD1 | 3.873648961 | 0.00057762 |
| Neprilysin | MME | 3.229970932 | 0.000944198 |
| UTP--glucose-1-phosphate | UGP2 | 3.02215004 | 0.001529127 |
| uridylyltransferase | | | |
| Major vault protein | MVP | 2.45493571 | 0.000536644 |
| Gamma-enolase; Enolase | ENO2 | 2.359129588 | 0.000297653 |
| Niemann-Pick C1 protein | NPC1 | 2.298453013 | 0.002822243 |
| Myosin regulatory light chain 12B; Myosin | MYL12B; | 2.242603302 | 0.000882352 |
| regulatory light chain 12A | MYL12A | | |
| Coronin-1C; Coronin | CORO1C | 1.745135625 | 0.001159066 |
| T-complex protein 1 subunit alpha | TCP1 | 1.660648346 | 0.002936757 |
| Tubulin-specific chaperone A | TBCA | 1.310288747 | 0.001436331 |
| Protein spinster homolog 1 | SPNS1 | 1.075093587 | 0.001325142 |
| 14-3-3 protein epsilon | YWHAE | 0.795204798 | 0.000971929 |
| Syntaxin-4 | STX4 | 0.590283712 | 0.000162322 |
| Band 4.1-like protein 2 | EPB41L2 | −2.013690313 | 0.000239126 |
| Latent-transforming growth factor beta- | LTBP2 | −2.616281509 | 0.002945221 |
| binding protein 2 | | | |
| Procollagen-lysine,2-oxoglutarate 5- | PLOD1 | −3.127297719 | 0.002214353 |
| dioxygenase 1 | | | |
| Hemoglobin subunit beta; LVV-hemorphin-7; | HBB | −3.162877401 | 0.002488562 |
| Spinorphin | | | |
| Fibrillin-1 | FBN1 | −3.394845327 | 9.5844E−06 |
| Thrombospondin-2 | THBS2 | −3.841739019 | 0.002997381 |
| Fibulin-1 | FBLN1 | −4.651089986 | 0.001453623 |
| Versican core protein | VCAN | −5.202549616 | 0.000440219 |
| Leucine-rich alpha-2-glycoprotein | LRG1 | −5.707799911 | 0.000438009 |

TABLE 7 shows a list of exemplary differentially expressed cargo proteins in BM-MSC vs UC-MSC EVs

| | | Log2 | |
|---|---|---|---|
| Protein | Gene | Difference | p-Value |
| Fibrinogen gamma chain | FGG | 11.53893725 | 9.38E−06 |
| Ceruloplasmin | CP | 11.35622056 | 0.000525 |
| Complement C1r subcomponent; | C1R | 9.726323446 | 0.000698 |
| Complement C1r subcomponent heavy | | | |
| chain; Complement C1r subcomponent light | | | |
| chain | | | |
| LIM and senescent cell antigen-like- | LIMS1 | 8.137312571 | 0.000225 |
| containing domain protein 1 | | | |
| Plasminogen; Plasmin heavy chain | PLG | 8.039388021 | 1.72E−05 |
| A; Activation peptide; Angiostatin; Plasmin | | | |
| heavy chain A, short form; Plasmin light | | | |
| chain B | | | |
| Glia-derived nexin | SERPINE2 | 7.861179988 | 3.12E−05 |
| Apolipoprotein E | APOE | 7.647237142 | 4.8E−06 |
| Apolipoprotein B-100; Apolipoprotein B-48 | APOB | 7.464042664 | 1.89E−05 |
| Complement C3; Complement C3 beta | C3 | 6.910105387 | 2.04E−07 |
| chain; C3-beta-c; Complement C3 alpha | | | |
| chain; C3a anaphylatoxin; Acylation | | | |
| stimulating protein; Complement C3b alpha | | | |
| chain; Complement C3c alpha chain | | | |
| fragment 1; Complement C3dg fragment; | | | |
| Complement C3g fragment; Complement | | | |
| C3d fragment; Complement C3f fragment; | | | |
| Complement C3c alpha chain fragment 2 | | | |
| Integrin-linked protein kinase | ILK | 6.548437119 | 9.87E−06 |
| Leucine-rich alpha-2-glycoprotein | LRG1 | 6.444966634 | 6.38E−05 |
| Alpha-2-HS-glycoprotein; Alpha-2-HS- | AHSG | 6.309399923 | 0.002604 |
| glycoprotein chain A; Alpha-2-HS- | | | |
| glycoprotein chain B | | | |
| Transthyretin | TTR | 6.187903086 | 0.017308 |
| Serotransferrin | TF | 6.148430824 | 0.001637 |
| Rho GDP-dissociation inhibitor 2 | ARHGDIB | 6.028390249 | 0.000235 |
| Carboxypeptidase N catalytic chain | CPN1 | 5.818281174 | 0.000154 |
| Thrombospondin-1 | THBS1 | 5.608400345 | 4.41E−06 |
| Vitamin D-binding protein | GC | 5.078195572 | 0.000292 |
| Gelsolin | GSN | 4.99415652 | 4.45E−06 |
| Clusterin; Clusterin beta chain; Clusterin | CLU | 4.993111293 | 3.38E−05 |
| alpha chain; Clusterin | | | |
| Coactosin-like protein | COTL1 | 4.906246821 | 0.001098 |
| Cadherin-13 | CDH13 | 4.815336227 | 0.00014 |
| Tubulin alpha-4A chain | TUBA4A | 4.779610634 | 0.001251 |
| EMILIN-1 | EMILIN1 | 4.644694646 | 0.00025 |
| Carboxypeptidase N subunit 2 | CPN2 | 4.516239166 | 0.000221 |
| Procollagen-lysine,2-oxoglutarate 5- | PLOD1 | 4.506978989 | 1.49E−05 |
| dioxygenase 1 | | | |
| Alpha-2-macroglobulin | A2M | 4.334324519 | 3.21E−05 |
| Prothrombin; Activation peptide fragment 1; | F2 | 4.287651062 | 8.7E−05 |
| Activation peptide fragment 2; Thrombin light | | | |
| chain; Thrombin heavy chain | | | |
| Hemoglobin subunit beta; LVV-hemorphin- | HBB | 4.274690628 | 0.000505 |
| 7; Spinorphin | | | |
| Talin-1 | TLN1 | 4.061203639 | 1.01E−05 |
| Complement component C8 beta chain | C8B | 3.717856089 | 0.000155 |
| Apolipoprotein A-II; Proapolipoprotein A-II; | APOA2 | 3.50073115 | 0.024092 |
| Truncated apolipoprotein A-II | | | |
| Alpha-1B-glycoprotein | A1BG | 3.405494054 | 0.003987 |
| Kininogen-1; Kininogen-1 heavy chain; T- | KNG1 | 3.401656151 | 0.017381 |
| kinin; Bradykinin; Lysyl-bradykinin; | | | |
| Kininogen-1 light chain; Low molecular | | | |
| weight growth-promoting factor | | | |
| Protein disulfide-isomerase A3 | PDIA3 | 3.274178823 | 0.000239 |
| Peptidyl-prolyl cis-trans isomerase B | PPIB | 3.256006877 | 0.000194 |
| Protein disulfide-isomerase A3 | PDIA3 | 3.13722229 | 0.000408 |
| Ras suppressor protein 1 | RSU1 | 3.120337168 | 0.003762 |
| Staphylococcal nuclease domain-containing | SND1 | 3.030210813 | 0.002457 |
| protein 1 | | | |
| Basement membrane-specific heparan | HSPG2 | 3.02191035 | 0.002672 |
| sulfate proteoglycan core protein; | | | |
| Endorepellin; LG3 peptide | | | |
| Ras-related protein Rab-6B | RAB6B | 2.949335734 | 0.009257 |
| Fibrillin-1 | FBN1 | 2.802187602 | 5.1E−05 |
| Latent-transforming growth factor beta- | LTBP1 | 2.769350052 | 0.003056 |
| binding protein 1 | | | |
| Filamin-A | FLNA | 2.759649277 | 0.000418 |
| N-acetylmuramoyl-L-alanine amidase | PGLYRP2 | 2.717362722 | 0.032715 |
| PDZ and LIM domain protein 7 | PDLIM7 | 2.668416023 | 0.008667 |
| Nidogen-1 | NID1 | 2.606767654 | 0.004205 |
| Coronin-1B; Coronin | CORO1B | 2.552958488 | 0.000146 |
| Transforming growth factor-beta-induced | TGFBI | 2.52694575 | 0.000388 |
| protein ig-h3 | | | |
| Fibulin-1 | FBLN1 | 2.46722285 | 0.066191 |
| Phosphatidylinositol 5-phosphate 4-kinase | PIP4K2A | 2.392644882 | 0.033541 |
| type-2 alpha | | | |
| Ras-related protein Rab-11A; Ras-related | RAB11A; RAB11B | 2.383615494 | 0.008702 |
| protein Rab-11B | | | |
| Neutral alpha-glucosidase AB | GANAB | 2.35820961 | 0.00012 |
| Nucleosome assembly protein 1-like 1 | NAP1L1 | 2.217396418 | 0.000266 |
| Integrin alpha-2 | ITGA2 | 2.217300097 | 0.02589 |
| Profilin-1 | PFN1 | 2.117497126 | 0.001646 |
| Adenine phosphoribosyltransferase | APRT | 2.05325826 | 0.001842 |
| Nidogen-2 | NID2 | 2.033540408 | 0.00345 |
| Calponin-2; Calponin | CNN2 | 2.02983729 | 0.00614 |
| Lysyl oxidase homolog 2 | LOXL2 | 2.026821136 | 0.018291 |
| Ras-related protein Rap-1b; Ras-related | RAP1B | 2.011583964 | 0.000669 |
| protein Rap-1b-like protein | | | |
| Alpha-actinin-1 | ACTN1 | 1.964907328 | 0.016346 |
| WD repeat-containing protein 1 | WDR1 | 1.94318072 | 0.001614 |
| Calreticulin | CALR | 1.917475382 | 0.019895 |
| Versican core protein | VCAN | 1.904214223 | 0.020251 |
| Drebrin-like protein | DBNL | 1.903315226 | 0.001995 |
| Four and a half LIM domains protein 1 | FHL1 | 1.892479579 | 0.025879 |
| CD9 antigen; Tetraspanin | CD9 | 1.826656977 | 0.052494 |
| Actin-related protein 2/3 complex subunit 5 | ARPC5 | 1.819590886 | 0.006237 |
| Haptoglobin; Haptoglobin alpha | HP | 1.599509557 | 0.073288 |
| chain; Haptoglobin beta chain | | | |
| Fibulin-1 | FBLN1 | 1.559761047 | 0.07549 |
| Ras-related protein Rap-1A | RAP1A | 1.520785014 | 0.027337 |
| EH domain-containing protein 3 | EHD3 | 1.503062566 | 0.017028 |
| Olfactomedin-like protein 3 | OLFML3 | 1.496929804 | 0.036128 |
| 78 kDa glucose-regulated protein | HSPA5 | 1.493914922 | 0.003278 |
| Transgelin-2 | TAGLN2 | 1.479733785 | 0.012948 |
| Coronin-1C; Coronin | CORO1C | 1.449632009 | 0.004079 |
| Metalloproteinase inhibitor 1 | TIMP1 | 1.418584506 | 0.055184 |
| Endoplasmin | HSP90B1 | 1.414442062 | 0.008221 |
| Septin-7 | SEPT7 | 1.366061529 | 0.050183 |
| EGF-like repeat and discoidin I-like domain- | EDIL3 | 1.341881434 | 0.026873 |
| containing protein 3 | | | |
| Heat shock 70 kDa protein 6; Putative heat | HSPA6; HSPA7 | 1.239110947 | 0.068452 |
| shock 70 kDa protein 7 | | | |
| Uroporphyrinogen decarboxylase | UROD | 1.1327432 | 0.086842 |
| Actin, cytoplasmic 2; Actin, cytoplasmic 2, N- | ACTG1 | 1.119582494 | 0.000878 |
| terminally processed | | | |
| Chloride intracellular channel protein 1 | CLIC1 | 1.111235301 | 0.000765 |
| Protein-L-isoaspartate(D-aspartate) O- | PCMT1 | 1.093972524 | 0.054207 |
| methyltransferase; Protein-L-isoaspartate O- | | | |
| methyltransferase | | | |
| Actin-related protein 2/3 complex subunit 3 | ARPC3 | 1.021934509 | 0.004881 |
| Ras-related protein Rab-8A | RAB8A | 1.011357625 | 0.077621 |
| Actin-related protein 2/3 complex subunit 4 | ARPC4; ARPC4-TTLL3 | 0.995695114 | 0.013535 |
| Actin, cytoplasmic 1; Actin, cytoplasmic 1, N- | ACTB | 0.99417305 | 0.042763 |
| terminally processed | | | |
| Glutathione S-transferase omega-1 | GSTO1 | 0.963256836 | 0.06289 |
| Elongation factor 1-gamma | EEF1G | 0.956480662 | 0.040932 |
| S-formylglutathione hydrolase | ESD | 0.938319524 | 0.03915 |
| Ubiquitin-conjugating enzyme E2 D3; | UBE2D3; UBE2D2 | 0.836124738 | 0.019979 |
| Ubiquitin-conjugating enzyme E2 D2 | | | |
| Actin, alpha cardiac muscle 1; Actin, alpha | ACTC1; ACTA1; ACTA2; | 0.813611984 | 0.007967 |
| skeletal muscle; Actin, aortic smooth muscle; | ACTG2 | | |
| Actin, gamma-enteric smooth muscle | | | |
| EH domain-containing protein 1 | EHD1 | 0.794841131 | 0.056673 |
| Xaa-Pro dipeptidase | PEPD | 0.743752797 | 0.00189 |
| Coatomer subunit alpha; Xenin; Proxenin | COPA | 0.671942393 | 0.048507 |
| Xaa-Pro aminopeptidase 1 | XPNPEP1 | 0.671688716 | 0.060397 |
| Peptidyl-prolyl cis-trans isomerase | PPIA | 0.650396983 | 0.050259 |
| A; Peptidyl-prolyl cis-trans isomerase A, N- | | | |
| terminally processed; Peptidyl-prolyl cis- | | | |
| trans isomerase | | | |
| Adenylyl cyclase-associated protein 1 | CAP1 | 0.641578674 | 0.024768 |
| Actin-related protein 2/3 complex subunit 1B | ARPC1B | 0.515665054 | 0.068968 |
| Actin-related protein 2 | ACTR2 | 0.505652746 | 0.069258 |
| Phosphoglucomutase-1 | PGM1 | 0.49863561 | 0.026697 |
| 14-3-3 protein eta | YWHAH | 0.463267644 | 0.027132 |
| Tetraspanin; Tetraspanin-4 | TSPAN4 | 0.372366587 | 0.093044 |
| Actin-related protein 3 | ACTR3 | 0.357124964 | 0.015024 |
| Ras-related C3 botulinum toxin substrate 1; | RAC1; RAC3 | −0.235600154 | 0.023612 |
| Ras-related C3 botulinum toxin substrate 3 | | | |
| Tubulin alpha-1B chain | TUBA1B | −0.256469091 | 0.06935 |
| Glyceraldehyde-3-phosphate | GAPDH | −0.43097496 | 0.031821 |
| dehydrogenase | | | |
| Annexin A7 | ANXA7 | −0.538093567 | 0.05981 |
| Rho GDP-dissociation inhibitor 1 | ARHGDIA | −0.550912857 | 0.069826 |
| Serine/threonine-protein phosphatase 2A | PPP2CA; PPP2CB | −0.586080551 | 0.019591 |
| catalytic subunit alpha isoform; | | | |
| Serine/threonine-protein phosphatase 2A | | | |
| catalytic subunit beta isoform | | | |
| Protein Wnt-5a; Protein Wnt | WNT5A | −0.658051173 | 0.033948 |
| F-actin-capping protein subunit alpha-1 | CAPZA1 | −0.696228027 | 0.003685 |
| Integrin alpha-3; Integrin alpha-3 heavy | ITGA3 | −0.742219925 | 0.071423 |
| chain; Integrin alpha-3 light chain | | | |
| Cysteine and glycine-rich protein 2 | CSRP2 | −0.758712133 | 0.043892 |
| Tubulin beta-4B chain | TUBB4B | −0.76170222 | 0.007693 |
| Triosephosphate isomerase | TPI1 | −0.793954849 | 0.037918 |
| 26S proteasome non-ATPase regulatory | PSMD7 | −0.808287938 | 0.07835 |
| subunit 7 | | | |
| Destrin | DSTN | −0.823013306 | 0.010363 |
| Ezrin | EZR | −0.85598882 | 0.01583 |
| 60S ribosomal protein L10a | RPL10A | −0.857755661 | 0.046063 |
| Proteasome subunit alpha type-5 | PSMA5 | −0.892100016 | 0.067697 |
| Gamma-enolase; Enolase | ENO2 | −0.906215668 | 0.022791 |
| Protein disulfide-isomerase | P4HB | −0.910208384 | 0.069507 |
| Basigin | BSG | −0.914412181 | 0.087364 |
| Ras-related protein Rab-1A | RAB1A | −0.920277913 | 0.0105 |
| Actin-related protein 2/3 complex subunit 1A | ARPC1A | −0.9428943 | 0.002192 |
| Ras-related protein Rab-7a | RAB7A | −0.953426361 | 0.002275 |
| Cell division control protein 42 homolog | CDC42 | −0.964885076 | 0.001601 |
| Programmed cell death 6-interacting protein | PDCD6IP | −0.984692891 | 0.071756 |
| Ras-related protein Rab-14 | RAB14 | −0.987318675 | 0.046321 |
| Phospholipid scramblase 3 | PLSCR3; TMEM256- | −1.004420916 | 0.042603 |
| | PLSCR3 | | |
| ATP synthase subunit beta, mitochondrial; | ATP5B | −1.008117676 | 0.003831 |
| ATP synthase subunit beta | | | |
| Transforming protein RhoA | RHOA | −1.008288066 | 3.05E−06 |
| Ras-related protein Rab-5C | RAB5C | −1.017657598 | 0.084024 |
| T-complex protein 1 subunit zeta | CCT6A | −1.018390656 | 0.081083 |
| Zinc transporter ZIP14 | SLC39A14 | −1.023371379 | 0.0677 |
| 1,4-alpha-glucan-branching enzyme | GBE1 | −1.025686264 | 0.060021 |
| SH3 domain-binding glutamic acid-rich-like | SH3BGRL3 | −1.026795069 | 0.065859 |
| protein 3 | | | |
| Heat shock protein HSP 90-alpha | HSP90AA1 | −1.043341955 | 0.013367 |
| T-complex protein 1 subunit gamma | CCT3 | −1.048494975 | 0.006036 |
| 72 kDa type IV collagenase; PEX | MMP2 | −1.050679525 | 0.081578 |
| Alpha-enolase | ENO1 | −1.062243144 | 0.006354 |
| Neutral amino acid transporter B(0); Amino | SLC1A5 | −1.069807053 | 0.079032 |
| acid transporter | | | |
| Rab GDP dissociation inhibitor beta | GDI2 | −1.082639058 | 0.023091 |
| Tubulin beta-8 chain | TUBB8 | −1.095911662 | 0.00154 |
| 40S ribosomal protein S11 | RPS11 | −1.117990494 | 0.00104 |
| T-complex protein 1 subunit theta | CCT8 | −1.123994191 | 0.001819 |
| Protein eva-1 homolog B | EVA1B | −1.151824315 | 0.059246 |
| Histidine triad nucleotide-binding protein 1 | HINT1 | −1.154263179 | 0.017157 |
| Procollagen C-endopeptidase enhancer 1 | PCOLCE | −1.159313838 | 0.006193 |
| T-complex protein 1 subunit delta | CCT4 | −1.171791712 | 0.045723 |
| Fascin | FSCN1 | −1.176886876 | 0.018236 |
| Septin-11 | SEPT11 | −1.195652008 | 0.003702 |
| Metalloproteinase inhibitor 2 | TIMP2 | −1.201161702 | 0.013401 |
| Syntenin-1 | SDCBP | −1.208391825 | 0.056188 |
| 14-3-3 protein gamma; 14-3-3 protein | YWHAG | −1.218238831 | 0.001209 |
| gamma, N-terminally processed | | | |
| Elongation factor 2 | EEF2 | −1.222723643 | 0.000477 |
| Tubulin beta-2A chain | TUBB2A | −1.231826464 | 0.023497 |
| Heterogeneous nuclear ribonucleoprotein K | HNRNPK | −1.240187327 | 0.000637 |
| Annexin A1; Annexin | ANXA1 | −1.264520009 | 0.005948 |
| Myosin-9 | MYH9 | −1.270516713 | 0.020816 |
| Band 4.1-like protein 2 | EPB41L2 | −1.284753482 | 0.012226 |
| Profilin-2; Profilin | PFN2 | −1.295940399 | 0.091851 |
| CD59 glycoprotein | CD59 | −1.307412465 | 0.019534 |
| Heat shock protein HSP 90-beta | HSP90AB1 | −1.318971634 | 0.003781 |
| Disintegrin and metalloproteinase domain- | ADAM10 | −1.324214935 | 0.065442 |
| containing protein 10 | | | |
| Alcohol dehydrogenase class-3 | ADH5 | −1.356280327 | 0.029018 |
| L-lactate dehydrogenase B chain; L-lactate | LDHB | −1.373849233 | 0.024359 |
| dehydrogenase | | | |
| Glucose-6-phosphate isomerase | GPI | −1.397775014 | 0.008802 |
| Heterogeneous nuclear ribonucleoprotein Q | SYNCRIP | −1.399332364 | 0.000797 |
| Proteasome subunit beta type-6; Proteasome | PSMB6 | −1.443112055 | 0.091775 |
| subunit beta type | | | |
| Moesin | MSN | −1.443145116 | 0.005359 |
| Chloride intracellular channel protein 4 | CLIC4 | −1.451898575 | 0.037067 |
| CD44 antigen | CD44 | −1.466406504 | 0.01546 |
| Human leukocyte antigen-A | HLA-A | −1.477623622 | 0.029491 |
| Gap junction alpha-1 protein | GJA1 | −1.485594432 | 0.000789 |
| Phosphatidylethanolamine-binding protein 1; | PEBP1 | −1.492188454 | 0.000434 |
| Hippocampal cholinergic neurostimulating | | | |
| peptide | | | |
| Serine--tRNA ligase, cytoplasmic | SARS | −1.492766698 | 0.004553 |
| Monocarboxylate transporter 1 | SLC16A1 | −1.50379022 | 0.005728 |
| Copine-1 | CPNE1 | −1.510477384 | 0.023687 |
| Ras-related protein Rab-21 | RAB21 | −1.511221568 | 0.005957 |
| T-complex protein 1 subunit eta | CCT7 | −1.515151978 | 0.034264 |
| 60S ribosomal protein L11 | RPL11 | −1.519344966 | 0.012229 |
| Histone H1.4; Histone H1.3 | HIST1H1E; HIST1H1D | −1.534877141 | 0.015207 |
| Histone H2B type 1-H; Histone H2B type 1- | HIST1H2BH; | −1.549941381 | 0.002521 |
| N; Histone H2B type 1-C/E/F/G/l; Histone | HIST1H2BN; | | |
| H2B type 1-D; Histone H2B type 1-K; Histone | HIST1H2BC; HIST1H2BD; | | |
| H2B type 2-F; Histone H2B type 1-M; | HIST1H2BK; | | |
| Histone H2B type F-S; Histone H2B; Histone | HIST2H2BF; | | |
| H2B type 1-L | HIST1H2BM; H2BFS; | | |
| | HIST1H2BL | | |
| Phosphoglycerate mutase 1 | PGAM1 | −1.558860143 | 0.001088 |
| Solute carrier family 43 member 3 | SLC43A3 | −1.561822573 | 0.002126 |
| Plectin | PLEC | −1.59133021 | 0.038458 |
| TLD domain-containing protein 1 | TLDC1 | −1.602067629 | 0.014368 |
| Ras-related protein R-Ras | RRAS | −1.612167358 | 0.041831 |
| Proteasome subunit alpha type- | PSMA2 | −1.612372716 | 0.013922 |
| 2; Proteasome subunit alpha type | | | |
| Neuropilin-1 | NRP1 | −1.615470886 | 0.003707 |
| Guanine nucleotide-binding protein | GNB1 | −1.619574865 | 0.001586 |
| G(I)/G(S)/G(T) subunit beta-1 | | | |
| DnaJ homolog subfamily A member 2 | DNAJA2 | −1.628042857 | 0.003349 |
| Guanine nucleotide-binding protein G(i) | GNAI2 | −1.632598241 | 0.004976 |
| subunit alpha-2 | | | |
| Voltage-dependent calcium channel subunit | CACNA2D1 | −1.637768428 | 0.007555 |
| alpha-2/delta-1; Voltage-dependent calcium | | | |
| channel subunit alpha-2-1; Voltage- | | | |
| dependent calcium channel subunit delta-1 | | | |
| Histone H2A type 1-C; Histone H2A type | HIST1H2AC; HIST3H2A; | −1.646881739 | 0.067189 |
| 3; Histone H2A type 1-B/E | HIST1H2AB | | |
| T-complex protein 1 subunit alpha | TCP1 | −1.662612915 | 0.006654 |
| Pregnancy zone protein | PZP | −1.698042552 | 0.03878 |
| Inactive tyrosine-protein kinase 7 | PTK7 | −1.718339284 | 0.00668 |
| Guanine nucleotide-binding protein | GNB2 | −1.722396851 | 0.022352 |
| G(I)/G(S)/G(T) subunit beta-2 | | | |
| V-type proton ATPase catalytic subunit A | ATP6V1A | −1.731585185 | 0.004611 |
| Histone H3.1 | HIST1H3A | −1.735469182 | 0.017473 |
| Catenin beta-1 | CTNNB1 | −1.746927261 | 0.029684 |
| CD99 antigen | CD99 | −1.792532603 | 0.057505 |
| Golgi-associated plant pathogenesis-related | GLIPR2 | −1.835244497 | 0.042592 |
| protein 1 | | | |
| Ephrin type-A receptor 2 | EPHA2 | −1.85188357 | 0.00989 |
| 40S ribosomal protein S18 | RPS18 | −1.864254634 | 0.019016 |
| Annexin A11 | ANXA11 | −1.88263003 | 0.00666 |
| AP-2 complex subunit alpha-1 | AP2A1 | −1.891040802 | 0.003834 |
| Large neutral amino acids transporter small | SLC7A5 | −1.896438599 | 0.008984 |
| subunit 1 | | | |
| L-lactate dehydrogenase A chain | LDHA | −1.931990941 | 0.000355 |
| Ubiquitin-conjugating enzyme E2 N | UBE2N | −1.946130753 | 0.029991 |
| Ras-related protein Rab-8B | RAB8B | −2.017372767 | 0.001921 |
| Sodium/potassium-transporting ATPase | ATP1A1 | −2.038682302 | 0.001639 |
| subunit alpha-1 | | | |
| Collagen alpha-2(I) chain | COL1A2 | −2.04159228 | 0.002909 |
| Membrane cofactor protein | CD46 | −2.063545545 | 0.002485 |
| Catenin alpha-1 | CTNNA1 | −2.063547134 | 0.023938 |
| Fructose-bisphosphate aldolase A; | ALDOA | −2.084307988 | 0.003307 |
| Fructose-bisphosphate aldolase | | | |
| AP-2 complex subunit mu | AP2M1 | −2.100608508 | 0.036783 |
| Metallothionein-2; Metallothionein- | MT2A; MT1X; MT1G | −2.120922724 | 0.001812 |
| 1X; Metallothionein-1G | | | |
| Clathrin heavy chain 1; Clathrin heavy chain | CLTC | −2.1215566 | 0.002412 |
| Tubulin beta-3 chain | TUBB3 | −2.166651408 | 0.016448 |
| Protein S100-A11; Protein S100-A11, N- | S100A11 | −2.16773351 | 0.014476 |
| terminally processed | | | |
| Ragulator complex protein LAMTOR1 | LAMTOR1 | −2.176279704 | 0.067025 |
| Malate dehydrogenase, cytoplasmic | MDH1 | −2.187123617 | 0.003051 |
| 14-3-3 protein beta/alpha; 14-3-3 protein | YWHAB | −2.190735499 | 0.008636 |
| beta/alpha, N-terminally processed | | | |
| Fibronectin; Anastellin; Ugl-Y1 ;Ugl-Y2; Ugl- | FN1 | −2.191310883 | 0.000311 |
| Y3 | | | |
| GTPase NRas | NRAS | −2.219266891 | 0.014678 |
| Integrin alpha-V; Integrin alpha-V heavy | ITGAV | −2.253095627 | 0.002372 |
| chain; Integrin alpha-V light chain | | | |
| Elongation factor 1-alpha 1; Putative | EEF1A1; EEF1A1P5 | −2.314221064 | 0.000316 |
| elongation factor 1-alpha-like 3; Elongation | | | |
| factor 1-alpha | | | |
| Isocitrate dehydrogenase [NADP] | IDH1 | −2.318430583 | 0.019377 |
| cytoplasmic | | | |
| 40S ribosomal protein S3 | RPS3 | −2.33172226 | 0.003216 |
| Protein S100-A16 | S100A16 | −2.341890971 | 0.002934 |
| Target of Nesh-SH3 | ABI3BP | −2.352123578 | 0.01635 |
| Protein deglycase DJ-1 | PARK7 | −2.355499268 | 0.061434 |
| Guanine nucleotide-binding protein subunit | GNB2L1 | −2.429574013 | 0.051818 |
| beta-2-like 1; Guanine nucleotide-binding | | | |
| protein subunit beta-2-like 1, N-terminally | | | |
| processed | | | |
| N(G),N(G)-dimethylarginine | DDAH2 | −2.429575602 | 0.00318 |
| dimethylaminohydrolase 2 | | | |
| 60S ribosomal protein L30 | RPL30 | −2.432015101 | 0.002761 |
| Anoctamin-6 | ANO6 | −2.454902649 | 0.016753 |
| Importin subunit beta-1 | KPNB1 | −2.460813522 | 0.015913 |
| Protein PTHB1 | BBS9 | −2.461169561 | 0.003939 |
| Myosin light polypeptide 6 | MYL6 | −2.4678847 | 0.000818 |
| Ras-related protein Rab-2A; Ras-related | RAB2A; RAB2B | −2.469094594 | 0.003118 |
| protein Rab-2B | | | |
| Protein S100-A6; Protein S100 | S100A6 | −2.492087046 | 0.00205 |
| Guanine nucleotide-binding protein subunit | GNA13 | −2.511872292 | 0.033683 |
| alpha-13 | | | |
| ATP-citrate synthase | ACLY | −2.523176829 | 0.004939 |
| Dihydropyrimidinase-related protein 3 | DPYSL3 | −2.549312592 | 0.002791 |
| Annexin A5; Annexin | ANXA5 | −2.55663681 | 0.000199 |
| Histone H4 | HIST1H4A | −2.576080322 | 0.016631 |
| Annexin A2; Annexin; Putative annexin A2- | ANXA2; ANXA2P2 | −2.613524119 | 0.002558 |
| like protein | | | |
| 5-nucleotidase | NT5E | −2.633281708 | 0.00045 |
| 40S ribosomal protein S7 | RPS7 | −2.6686999 | 0.026754 |
| Collagen alpha-1(I) chain | COL1A1 | −2.674326579 | 0.004696 |
| Histone H2A type 1; Histone H2A type 1- | HIST1H2AG; HIST1H2AJ; | −2.676062266 | 0.014559 |
| J; Histone H2A.J; Histone H2A type 1-H; | H2AFJ; HIST1H2AH; | | |
| Histone H2A type 1-D; Histone H2A | HIST1H2AD | | |
| Trophoblast glycoprotein | TPBG | −2.765989304 | 0.003007 |
| Catenin delta-1 | CTNND1 | −2.793895086 | 0.000197 |
| Myeloid-associated differentiation marker | MYADM | −2.79438146 | 0.007036 |
| Hemoglobin subunit alpha | HBA1 | −2.79531606 | 0.005925 |
| Transitional endoplasmic reticulum ATPase | VCP | −2.804110209 | 0.000151 |
| Rho-related GTP-binding protein RhoG | RHOG | −2.82045428 | 8.83E−05 |
| Unconventional myosin-Ic | MYO1C | −2.825150808 | 0.008511 |
| Guanine nucleotide-binding protein | GNG5 | −2.839602788 | 0.019566 |
| G(I)/G(S)/G(O) subunit gamma-5 | | | |
| Guanine nucleotide-binding protein G(k) | GNAI3 | −2.943008741 | 0.0376 |
| subunit alpha | | | |
| EH domain-containing protein 4 | EHD4 | −2.944389343 | 0.012516 |
| Brain acid soluble protein 1 | BASP1 | −3.048605601 | 0.000922 |
| 14-3-3 protein epsilon | YWHAE | −3.104934692 | 0.007848 |
| Ras-related protein Ral-A | RALA | −3.132036845 | 0.000447 |
| Monocarboxylate transporter 4 | SLC16A3 | −3.139686584 | 0.001014 |
| Dihydropyrimidinase-related protein 2 | DPYSL2 | −3.168667475 | 0.000687 |
| Matrix metalloproteinase-14 | MMP14 | −3.185950597 | 0.000533 |
| Annexin A6; Annexin | ANXA6 | −3.196523031 | 1.55E−05 |
| ADP-ribosylation factor-like protein 8B | ARL8B | −3.351240794 | 0.00235 |
| Thymosin beta-4; Hematopoietic system | TMSB4X | −3.377904256 | 0.005972 |
| regulatory peptide | | | |
| Integrin beta-5 | ITGB5 | −3.385969798 | 0.004883 |
| Ubiquitin carboxyl-terminal hydrolase | UCHL1 | −3.423197428 | 0.002138 |
| isozyme L1; Ubiquitin carboxyl-terminal | | | |
| hydrolase | | | |
| Prolyl endopeptidase FAP; Antiplasmin- | FAP | −3.437456449 | 0.076019 |
| cleaving enzyme FAP, soluble form | | | |
| Galectin-1 | LGALS1 | −3.437585831 | 0.001447 |
| Ribonuclease inhibitor | RNH1 | −3.524065653 | 0.008254 |
| MARCKS-related protein | MARCKSL1 | −3.526751836 | 0.006391 |
| Plasma membrane calcium-transporting | ATP2B1 | −3.596431096 | 0.015383 |
| ATPase 1; Calcium-transporting ATPase | | | |
| Major vault protein | MVP | −3.74001503 | 0.003364 |
| Neprilysin | MME | −3.786698023 | 3.33E−05 |
| Nucleoside diphosphate kinase; Nucleoside | NME1-NME2; NME2 | −3.951183319 | 0.002235 |
| diphosphate kinase B | | | |
| Transferrin receptor protein 1; Transferrin | TFRC | −3.952521642 | 0.000586 |
| receptor protein 1, serum form | | | |
| D-3-phosphoglycerate dehydrogenase | PHGDH | −3.960495631 | 0.000408 |
| Histone H1.5 | HIST1H1B | −4.089924494 | 0.0022 |
| Fatty acid synthase; [Acyl-carrier-protein] S- | FASN | −4.139530182 | 0.006257 |
| acetyltransferase; [Acyl-carrier-protein] S- | | | |
| malonyltransferase; 3-oxoacyl-[acyl-carrier- | | | |
| protein] synthase; 3-oxoacyl-[acyl-carrier- | | | |
| protein] reductase; 3-hydroxyacyl-[acyl- | | | |
| carrier-protein] dehydratase; Enoyl-[acyl- | | | |
| carrier-protein] reductase; Oleoyl-[acyl- | | | |
| carrier-protein] hydrolase | | | |
| Annexin A4; Annexin | ANXA4 | −4.170916875 | 0.002138 |
| Synaptic vesicle membrane protein VAT-1 | VAT1 | −4.173790614 | 0.001159 |
| homolog | | | |
| 60S acidic ribosomal protein P0; 60S acidic | RPLPO; RPLPOP6 | −4.195742289 | 0.004046 |
| ribosomal protein P0-like | | | |
| Myristoylated alanine-rich C-kinase | MARCKS | −4.222830455 | 0.002064 |
| substrate | | | |
| Ras-related protein Rap-2b | RAP2B | −4.480142593 | 0.000401 |
| Vimentin | VIM | −4.69357427 | 0.000463 |
| Prolow-density lipoprotein receptor-related | LRP1 | −4.759888967 | 0.000379 |
| protein 1; Low-density lipoprotein receptor- | | | |
| related protein 1 85 kDa subunit; Low-density | | | |
| lipoprotein receptor-related protein 1 515 | | | |
| kDa subunit; Low-density lipoprotein | | | |
| receptor-related protein 1 intracellular | | | |
| domain | | | |
| Tropomyosin alpha-4 chain | TPM4 | −4.999219259 | 0.000459 |
| Transketolase | TKT | −5.062716802 | 0.000144 |
| Serpin H1 | SERPINH1 | −5.194419225 | 0.002085 |
| Niban-like protein 1 | FAM129B | −5.565500259 | 1.52E−06 |
| UTP--glucose-1-phosphate | UGP2 | −5.668725332 | 4.9E−05 |
| uridylyltransferase | | | |
| Neuroblast differentiation-associated protein | AHNAK | −6.436819077 | 0.006955 |
| AHNAK | | | |
| Myoferlin | MYOF | −7.318937937 | 0.000588 |

TABLE 8 shows a list of exemplary differentially expressed cargo proteins in HOC vs UC-MSC EVs;

| Protein | Gene | Log2 Difference | p-Value |
|---|---|---|---|
| Fibrinogen beta chain; Fibrinopeptide B; | FGB | 11.11058807 | 7.92E−05 |
| Fibrinogen beta chain | | | |
| Inter-alpha-trypsin inhibitor heavy chain H4; | ITIH4 | 9.603206952 | 0.000379 |
| 70 kDa inter-alpha-trypsin inhibitor heavy | | | |
| chain H4; 35 kDa inter-alpha-trypsin inhibitor | | | |
| heavy chain H4 | | | |
| Alpha-1-antitrypsin; Short peptide from AAT | SERPINA1 | 9.431540171 | 0.000579 |
| Vitronectin; Vitronectin V65 subunit; | VTN | 8.951140722 | 0.000311 |
| Vitronectin V10 subunit; Somatomedin-B | | | |
| Complement component C9; Complement | C9 | 8.426261266 | 0.000894 |
| component C9a; Complement component C9b | | | |
| Complement C1s subcomponent; | C1S | 8.376797358 | 8.98E−06 |
| Complement C1s subcomponent heavy | | | |
| chain; Complement C1s subcomponent light | | | |
| chain | | | |
| Ceruloplasmin | CP | 8.364689509 | 0.000183 |
| Apolipoprotein B-100; Apolipoprotein B-48 | APOB | 8.327229182 | 0.000175 |
| LIM and senescent cell antigen-like- | LIMS1 | 8.326121012 | 0.000204 |
| containing domain protein 1 | | | |
| Complement C1r subcomponent; | C1R | 7.75058492 | 0.000519 |
| Complement C1r subcomponent heavy | | | |
| chain; Complement C1r subcomponent light | | | |
| chain | | | |
| Complement C4-B; Complement C4 beta | C4B | 7.687116623 | 0.000154 |
| chain; Complement C4-B alpha chain; C4a | | | |
| anaphylatoxin; C4b-B; C4d-B; Complement | | | |
| C4 gamma chain | | | |
| Plasminogen; Plasmin heavy chain | PLG | 7.499070485 | 2.33E−05 |
| A; Activation peptide; Angiostatin; Plasmin | | | |
| heavy chain A, short form; Plasmin light | | | |
| chain B | | | |
| Integrin-linked protein kinase | ILK | 7.381437302 | 0.000545 |
| Apolipoprotein E | APOE | 6.806344986 | 7.44E−06 |
| Complement C3; Complement C3 beta | C3 | 6.641883214 | 4E−06 |
| chain; C3-beta-c; Complement C3 alpha | | | |
| chain; C3a anaphylatoxin; Acylation | | | |
| stimulating protein; Complement C3b alpha | | | |
| chain; Complement C3c alpha chain | | | |
| fragment 1; Complement C3dg fragment; | | | |
| Complement C3g fragment; Complement | | | |
| C3d fragment; Complement C3f fragment; | | | |
| Complement C3c alpha chain fragment 2 | | | |
| Clusterin; Clusterin beta chain; Clusterin | CLU | 6.195671717 | 0.000395 |
| alpha chain; Clusterin | | | |
| Insulin-like growth factor-binding protein | IGFALS | 5.764334361 | 0.002602 |
| complex acid labile subunit | | | |
| Gelsolin | GSN | 5.757106781 | 6.06E−06 |
| Lumican | LUM | 5.490712484 | 0.001569 |
| Vitamin D-binding protein | GC | 5.337202708 | 0.000663 |
| Vasodilator-stimulated phosphoprotein | VASP | 5.0805734 | 0.010247 |
| Proto-oncogene tyrosine-protein kinase Src | SRC | 5.076328595 | 0.002997 |
| Four and a half LIM domains protein 1 | FHL1 | 4.98345534 | 0.001279 |
| Talin-1 | TLN1 | 4.691000621 | 4.14E−06 |
| Carboxypeptidase N catalytic chain | CPN1 | 4.573361079 | 0.002444 |
| Alpha-1-antichymotrypsin; Alpha-1- | SERPINA3 | 4.569726944 | 0.001565 |
| antichymotrypsin His-Pro-less | | | |
| Serotransferrin | TF | 4.560809453 | 0.004153 |
| Angiopoietin-related protein 4 | ANGPTL4 | 4.481006622 | 0.032783 |
| Complement C5; Complement C5 beta | C5 | 4.462275187 | 0.026043 |
| chain; Complement C5 alpha chain; C5a | | | |
| anaphylatoxin; Complement C5 alpha chain | | | |
| Filamin-A | FLNA | 4.293055852 | 9.3E−06 |
| Glia-derived nexin | SERPINE2 | 4.185921987 | 0.001963 |
| Haptoglobin-related protein | HPR | 4.115037282 | 0.001807 |
| Coactosin-like protein | COTL1 | 3.995031993 | 0.000296 |
| Peptidyl-prolyl cis-trans isomerase B | PPIB | 3.778753281 | 0.012164 |
| Thrombospondin-1 | THBS1 | 3.7745622 | 0.000192 |
| Tubulin alpha-4A chain | TUBA4A | 3.76786232 | 0.007346 |
| Carboxypeptidase N subunit 2 | CPN2 | 3.693068186 | 0.001597 |
| Tubulin beta-1 chain | TUBB1 | 3.602682749 | 0.048074 |
| Alpha-2-macroglobulin | A2M | 3.599001567 | 0.000632 |
| Prothrombin; Activation peptide fragment 1; | F2 | 3.46970431 | 0.000479 |
| Activation peptide fragment 2; Thrombin light | | | |
| chain; Thrombin heavy chain | | | |
| Ras suppressor protein 1 | RSU1 | 3.458136241 | 0.002814 |
| Protein disulfide-isomerase A3 | PDIA3 | 3.43069458 | 0.000112 |
| Sulfhydryl oxidase 1 | QSOX1 | 3.428002675 | 0.026098 |
| Staphylococcal nuclease domain-containing | SND1 | 3.324395498 | 0.000945 |
| protein 1 | | | |
| ADAMTS-like protein 1 | ADAMTSL1 | 3.27017053 | 0.042179 |
| Rho GDP-dissociation inhibitor 2 | ARHGDIB | 3.234182358 | 0.009995 |
| Coronin-1C; Coronin | CORO1C | 3.194767634 | 3.3E−05 |
| Transthyretin | TTR | 3.114487966 | 0.003372 |
| Calreticulin | CALR | 3.107383092 | 0.020328 |
| Actin-related protein 2/3 complex subunit 5 | ARPC5 | 2.948189418 | 0.049673 |
| Plasma protease C1 inhibitor | SERPING1 | 2.92954127 | 0.024518 |
| Uroporphyrinogen decarboxylase | UROD | 2.900180181 | 0.009588 |
| EH domain-containing protein 3 | EHD3 | 2.88543129 | 0.004383 |
| Calponin-2; Calponin | CNN2 | 2.862098058 | 0.005281 |
| Phosphatidylinositol-glycan-specific | GPLD1 | 2.857206027 | 0.04068 |
| phospholipase D | | | |
| Phosphatidylinositol 5-phosphate 4-kinase | PIP4K2A | 2.854852041 | 0.004301 |
| type-2 alpha | | | |
| Nucleosome assembly protein 1-like 1 | NAP1L1 | 2.83447202 | 0.0006 |
| Collagen alpha-1(XII) chain | COL12A1 | 2.79437383 | 0.010034 |
| Protein disulfide-isomerase A3 | PDIA3 | 2.775880814 | 0.00051 |
| Nidogen-2 | NID2 | 2.741861979 | 0.022596 |
| EGF-containing fibulin-like extracellular | EFEMP2 | 2.674822489 | 0.003763 |
| matrix protein 2 | | | |
| Pigment epithelium-derived factor | SERPINF1 | 2.582054774 | 0.000797 |
| Lysyl oxidase homolog 2 | LOXL2 | 2.493530909 | 0.048896 |
| Phosphatidylcholine-sterol acyltransferase | LCAT | 2.440402985 | 0.01618 |
| Alpha-actinin-1 | ACTN1 | 2.410259883 | 0.046168 |
| Profilin-1 | PFN1 | 2.332916896 | 0.004647 |
| Ras-related protein Rap-1b; Ras-related | RAP1B | 2.314561844 | 0.009431 |
| protein Rap-1b-like protein | | | |
| Glutathione S-transferase omega-1 | GSTO1 | 2.308996201 | 0.003941 |
| Endoplasmin | HSP90B1 | 2.247552236 | 0.001063 |
| WD repeat-containing protein 1 | WDR1 | 2.199163437 | 0.004992 |
| Ras-related C3 botulinum toxin substrate 2 | RAC2 | 2.159878413 | 0.015511 |
| Cysteine and glycine-rich protein 1 | CSRP1 | 2.123530706 | 0.005094 |
| Transforming growth factor-beta-induced | TGFBI | 2.085976283 | 0.000786 |
| protein ig-h3 | | | |
| Transgelin-2 | TAGLN2 | 2.038931529 | 0.020321 |
| Coagulation factor X; Factor X light chain; | F10 | 1.989002228 | 0.029992 |
| Factor X heavy chain; Activated factor Xa | | | |
| heavy chain | | | |
| Microfibrillar-associated protein 2 | MFAP2 | 1.916358948 | 0.016297 |
| Integrin alpha-6; Integrin alpha-6 heavy | ITGA6 | 1.90281868 | 0.016317 |
| chain; Integrin alpha-6 light chain; Processed | | | |
| integrin alpha-6 | | | |
| Actin, alpha cardiac muscle 1; Actin, alpha | ACTC1; ACTA1; | 1.864763896 | 0.004322 |
| skeletal muscle; Actin, aortic smooth muscle; | ACTA2; ACTG2 | | |
| Actin, gamma-enteric smooth muscle | | | |
| Actin, cytoplasmic 1; Actin, cytoplasmic 1, N- | ACTB | 1.826754888 | 0.025265 |
| terminally processed | | | |
| Actin-related protein 2/3 complex subunit 1B | ARPC1B | 1.813779831 | 0.009392 |
| Actin, cytoplasmic 2; Actin, cytoplasmic 2, N- | ACTG1 | 1.781508764 | 0.002245 |
| terminally processed | | | |
| Complement component C8 beta chain | C8B | 1.725031535 | 0.029478 |
| 60S ribosomal protein L12 | RPL12 | 1.66728274 | 0.005073 |
| 78 kDa glucose-regulated protein | HSPA5 | 1.644758224 | 0.000552 |
| Protein-L-isoaspartate(D-aspartate) O- | PCMT1 | 1.494677226 | 0.052821 |
| methyltransferase; Protein-L-isoaspartate O- | | | |
| methyltransferase | | | |
| Adenine phosphoribosyltransferase | APRT | 1.471345266 | 0.010296 |
| Beta-2-glycoprotein 1 | APOH | 1.462315877 | 0.055964 |
| Phosphoglucomutase-1 | PGM1 | 1.457342148 | 0.001126 |
| Gamma-enolase; Enolase | ENO2 | 1.45291392 | 0.001952 |
| Actin-related protein 2/3 complex subunit 4 | ARPC4; | 1.447724024 | 0.000529 |
| | ARPC4-TTLL3 | | |
| Neutral alpha-glucosidase AB | GANAB | 1.397813797 | 0.023614 |
| Procollagen-lysine,2-oxoglutarate 5- | PLOD1 | 1.379681269 | 0.033705 |
| dioxygenase 1 | | | |
| Actin-related protein 2/3 complex subunit 3 | ARPC3 | 1.369472504 | 0.014606 |
| Rab GDP dissociation inhibitor alpha | GDI1 | 1.368916829 | 0.025072 |
| Ras-related protein Rab-11A; Ras-related | RAB11A; RAB11B | 1.308354696 | 0.020607 |
| protein Rab-11B | | | |
| Peroxiredoxin-6 | PRDX6 | 1.286359787 | 0.000387 |
| Septin-7 | SEPTIN7 | 1.275589625 | 0.012999 |
| Septin-2 | SEPTIN2 | 1.272145589 | 0.060459 |
| Chloride intracellular channel protein 1 | CLIC1 | 1.262408574 | 0.000416 |
| Phosphoglycerate kinase 1 | PGK1 | 1.241307576 | 0.020908 |
| Latent-transforming growth factor beta- | LTBP1 | 1.203079224 | 0.00599 |
| binding protein 1 | | | |
| Hemoglobin subunit beta; LVV-hemorphin- | HBB | 1.111813227 | 0.007298 |
| 7; Spinorphin | | | |
| Xaa-Pro aminopeptidase 1 | XPNPEP1 | 1.107275009 | 0.012813 |
| Actin-related protein 2 | ACTR2 | 1.090003967 | 0.020213 |
| Heat shock protein beta-1 | HSPB1 | 1.074621836 | 0.046299 |
| Bifunctional purine biosynthesis protein PURH; | ATIC | 0.972947439 | 0.058821 |
| Phosphoribosylaminoimidazolecarboxamide | | | |
| formyltransferase; IMP cyclohydrolase | | | |
| F-actin-capping protein subunit alpha-2 | CAPZA2 | 0.947106679 | 0.003918 |
| Metalloproteinase inhibitor 1 | TIMP1 | 0.93580691 | 0.023824 |
| Ubiquitin-like modifier-activating enzyme 1 | UBA1 | 0.884912491 | 0.003884 |
| Peptidyl-prolyl cis-trans isomerase A; | PPIA | 0.863606771 | 0.023129 |
| Peptidyl-prolyl cis-trans isomerase A, N- | | | |
| terminally processed; Peptidyl-prolyl cis- | | | |
| trans isomerase | | | |
| Actin-related protein 3 | ACTR3 | 0.839741389 | 0.004516 |
| Elongation factor 1-gamma | EEF1G | 0.79719162 | 0.038323 |
| Alpha-2-HS-glycoprotein; Alpha-2-HS- | AHSG | 0.793289185 | 0.014843 |
| glycoprotein chain A; Alpha-2-HS- | | | |
| glycoprotein chain B | | | |
| Adenylyl cyclase-associated protein 1 | CAP1 | 0.765294393 | 0.019931 |
| Myosin-9 | MYH9 | 0.739054998 | 0.023288 |
| Complement C1r subcomponent-like protein | C1RL | 0.737073898 | 0.035242 |
| Alpha-centractin; Beta-centractin | ACTR1A; ACTR1B | 0.70118777 | 0.001145 |
| Procollagen-lysine, 2-oxoglutarate 5- | PLOD2 | 0.65877533 | 0.017883 |
| dioxygenase 2 | | | |
| ATP synthase subunit beta, mitochondrial; | ATP5B | 0.441438993 | 0.031448 |
| ATP synthase subunit beta | | | |
| Fibrillin-1 | FBN1 | −0.592657725 | 0.003432 |
| Cell division control protein 42 homolog | CDC42 | −0.596544266 | 0.005793 |
| 14-3-3 protein gamma; 14-3-3 protein | YWHAG | −0.647521973 | 0.035179 |
| gamma, N-terminally processed | | | |
| 4F2 cell-surface antigen heavy chain | SLC3A2 | −0.698338826 | 0.036403 |
| T-complex protein 1 subunit theta | CCT8 | −0.720439911 | 0.058868 |
| Heterogeneous nuclear ribonucleoprotein Q | SYNCRIP | −0.747550646 | 0.037248 |
| Elongation factor 2 | EEF2 | −0.779102961 | 0.009097 |
| V-type proton ATPase catalytic subunit A | ATP6V1A | −0.780028661 | 0.019082 |
| Transforming protein RhoA | RHOA | −0.797949473 | 0.051211 |
| Rab GDP dissociation inhibitor beta | GDI2 | −0.904081345 | 0.030878 |
| Integrin beta-1 | ITGB1 | −0.928953171 | 0.002827 |
| Zinc transporter ZIP14 | SLC39A14 | −0.95541509 | 0.041163 |
| Malate dehydrogenase, cytoplasmic | MDH1 | −0.972000758 | 0.047999 |
| Tetraspanin; Tetraspanin-4 | TSPAN4 | −0.975193342 | 2.94E−05 |
| Moesin | MSN | −0.996741613 | 0.026453 |
| Annexin A7 | ANXA7 | −1.027648926 | 0.006365 |
| Myosin light polypeptide 6 | MYL6 | −1.029092153 | 0.004521 |
| 5-nucleotidase | NT5E | −1.03784879 | 0.0468 |
| Fructose-bisphosphate aldolase A; Fructose- | ALDOA | −1.091207504 | 0.044502 |
| bisphosphate aldolase | | | |
| L-lactate dehydrogenase B chain; L-lactate | LDHB | −1.097032547 | 0.045997 |
| dehydrogenase | | | |
| 60S ribosomal protein L11 | RPL11 | −1.134010951 | 0.056249 |
| Serine--tRNA ligase, cytoplasmic | SARS | −1.184607188 | 0.026128 |
| Histone H2B type 1-H; Histone H2B type 1- | HIST1H2BH; | −1.188809713 | 0.035265 |
| N; Histone H2B type 1-C/E/F/G/I; Histone | HIST1H2BN; | | |
| H2B type 1-D; Histone H2B type 1-K; Histone | HIST1H2BC; | | |
| H2B type 2-F; Histone H2B type 1-M; Histone | HIST1H2BD; | | |
| H2B type F-S; Histone H2B; Histone H2B | HIST1H2BK; | | |
| type 1-L | HIST2H2BF; | | |
| | HIST1H2BM; | | |
| | H2BFS; | | |
| | HIST1H2BL | | |
| Heat shock protein HSP 90-beta | HSP90AB1 | −1.194969177 | 0.014259 |
| Inactive tyrosine-protein kinase 7 | PTK7 | −1.200071335 | 0.057536 |
| Ras-related protein Rab-7a | RAB7A | −1.226971308 | 0.015618 |
| Ezrin | EZR | −1.332111359 | 0.021188 |
| Vacuolar protein sorting-associated protein 35 | VPS35 | −1.346278191 | 0.039945 |
| TLD domain-containing protein 1 | TLDC1 | −1.348193169 | 0.039772 |
| Ras-related protein Rab-8B | RAB8B | −1.385351817 | 0.033773 |
| Elongation factor 1-alpha 1; Putative | EEF1A1; | −1.462671916 | 0.029055 |
| elongation factor 1-alpha-like 3; Elongation | EEF1A1P5 | | |
| factor 1-alpha | | | |
| Annexin A2; Annexin; Putative annexin A2- | ANXA2; | −1.465075175 | 0.036693 |
| like protein | ANXA2P2 | | |
| Haptoglobin; Haptoglobin alpha chain; | HP | −1.491697311 | 0.021116 |
| Haptoglobin beta chain | | | |
| FH1/FH2 domain-containing protein 1 | FHOD1 | −1.515716553 | 0.040192 |
| Guanine nucleotide-binding protein | GNB1 | −1.574413935 | 0.001649 |
| G(I)/G(S)/G(T) subunit beta-1 | | | |
| 40S ribosomal protein S3 | RPS3 | −1.575768789 | 0.03911 |
| CD59 glycoprotein | CD59 | −1.645769119 | 0.051675 |
| CD99 antigen | CD99 | −1.661611557 | 0.033678 |
| Hemopexin | HPX | −1.663476308 | 0.001689 |
| Peroxidasin homolog | PXDN | −1.677679698 | 0.055265 |
| Ras-related protein Rab-21 | RAB21 | −1.743112564 | 0.003943 |
| Prostaglandin F2 receptor negative regulator | PTGFRN | −1.751708984 | 0.012704 |
| Syndecan-Binding Protein | SDCBP | −1.755639394 | 0.014768 |
| Guanine nucleotide-binding protein G(i) | GNAI2 | −1.756656011 | 0.005896 |
| subunit alpha-2 | | | |
| Neutral amino acid transporter B(0); Amino | SLC1A5 | −1.765870412 | 0.020879 |
| acid transporter | | | |
| Integrin alpha-4 | ITGA4 | −1.775570552 | 0.04719 |
| Phospholipid transfer protein | PLTP | −1.777731578 | 0.056115 |
| ATP-citrate synthase | ACLY | −1.790583928 | 0.014228 |
| Ras-related protein Rab-35 | RAB35 | −1.794715881 | 0.036721 |
| Chloride intracellular channel protein 4 | CLIC4 | −1.85754331 | 0.029822 |
| 14-3-3 protein beta/alpha; 14-3-3 protein | YWHAB | −1.882423401 | 0.02082 |
| beta/alpha, N-terminally processed | | | |
| Agrin; Agrin N-terminal 110 kDa subunit; | AGRN | −1.885240555 | 0.049916 |
| Agrin C-terminal 110 kDa subunit; Agrin C- | | | |
| terminal 90 kDa fragment; Agrin C-terminal | | | |
| 22 kDa fragment | | | |
| Pentraxin-related protein PTX3 | PTX3 | −1.912286758 | 0.02723 |
| Ras-related protein R-Ras | RRAS | −1.947348277 | 0.052828 |
| GTPase NRas | NRAS | −1.951378504 | 0.046563 |
| Neuropilin-1 | NRP1 | −1.959346135 | 0.000393 |
| Catenin alpha-1 | CTNNA1 | −1.969868342 | 0.032565 |
| Guanine nucleotide-binding protein | GNB2 | −1.972418467 | 0.012085 |
| G(I)/G(S)/G(T) subunit beta-2 | | | |
| Sodium/potassium-transporting ATPase | ATP1A1 | −1.977364858 | 0.001579 |
| subunit alpha-1 | | | |
| Syntenin-1 | SDCBP | −2.013303757 | 0.00264 |
| Large neutral amino acids transporter small | SLC7A5 | −2.023668925 | 0.011874 |
| subunit 1 | | | |
| Guanine nucleotide-binding protein | GNG12 | −2.036747615 | 0.039289 |
| G(I)/G(S)/G(O) subunit gamma-12 | | | |
| 60S acidic ribosomal protein P0; 60S acidic | RPLP0; | −2.044466654 | 0.012194 |
| ribosomal protein P0-like | RPLP0P6 | | |
| Metallothionein-2; Metallothionein-1X; | MT2A; MT1X; | −2.053142548 | 0.028637 |
| Metallothionein-1G | MT1G | | |
| Ubiquitin-conjugating enzyme E2 N | UBE2N | −2.066952387 | 0.034247 |
| DnaJ homolog subfamily A member 2 | DNAJA2 | −2.071369171 | 0.004395 |
| Fibulin-1 | FBLN1 | −2.099943161 | 0.001976 |
| Programmed cell death 6-interacting protein | PDCD6IP | −2.110923131 | 0.000824 |
| Catenin beta-1 | CTNNB1 | −2.137156169 | 0.015436 |
| Integrin alpha-V; Integrin alpha-V heavy | ITGAV | −2.140857697 | 0.00378 |
| chain; Integrin alpha-V light chain | | | |
| Gap junction alpha-1 protein | GJA1 | −2.151175181 | 0.034367 |
| Annexin A5; Annexin | ANXA5 | −2.200419108 | 0.000631 |
| EGF-like repeat and discoidin I-like domain- | EDIL3 | −2.208995183 | 0.019798 |
| containing protein 3 | | | |
| Histone H2A type 1; Histone H2A type 1-J; | HIST1H2AG; | −2.256967545 | 0.040086 |
| Histone H2A.J; Histone H2A type 1-H; | HIST1H2AJ; | | |
| Histone H2A type 1-D; Histone H2A | H2AFJ; | | |
| | HIST1H2AH; | | |
| | HIST1H2AD | | |
| Tetraspanin-14; Tetraspanin | TSPAN14 | −2.263004303 | 0.009158 |
| Fibronectin; Anastellin; UgI-Y1; UgI-Y2; | FN1 | −2.296021779 | 0.000588 |
| UgI-Y3 | | | |
| 14-3-3 protein epsilon | YWHAE | −2.309729894 | 0.020688 |
| MARCKS-related protein | MARCKSL1 | −2.326803207 | 0.019604 |
| Guanine nucleotide-binding protein | GNG5 | −2.366711299 | 0.044132 |
| G(I)/G(S)/G(O) subunit gamma-5 | | | |
| Integrin alpha-5; Integrin alpha-5 heavy | ITGA5 | −2.400620778 | 0.017531 |
| chain; Integrin alpha-5 light chain | | | |
| Annexin A11 | ANXA11 | −2.425679525 | 0.003076 |
| Protein S100-A16 | S100A16 | −2.436091105 | 0.007104 |
| Ephrin type-A receptor 2 | EPHA2 | −2.443478902 | 0.013385 |
| Protein tweety homolog 3 | TTYH3 | −2.480733236 | 3.85E−06 |
| Brain acid soluble protein 1 | BASP1 | −2.483914057 | 0.004133 |
| Disintegrin and metalloproteinase domain- | ADAM10 | −2.48770078 | 0.000506 |
| containing protein 10 | | | |
| Protein S100-A11; Protein S100-A11, N- | S100A11 | −2.50687472 | 0.02623 |
| terminally processed | | | |
| Catenin delta-1 | CTNND1 | −2.507287343 | 0.000712 |
| Transitional endoplasmic reticulum ATPase | VCP | −2.516525904 | 0.000121 |
| Membrane cofactor protein | CD46 | −2.527350426 | 0.000127 |
| Rho-related GTP-binding protein RhoG | RHOG | −2.541114171 | 0.000127 |
| Protein eva-1 homolog B | EVA1B | −2.564523697 | 0.000199 |
| Voltage-dependent calcium channel subunit | CACNA2D1 | −2.571592331 | 0.017656 |
| alpha-2/delta-1; Voltage-dependent calcium | | | |
| channel subunit alpha-2-1; Voltage- | | | |
| dependent calcium channel subunit delta-1 | | | |
| Ribonuclease inhibitor | RNH1 | −2.594710032 | 0.031051 |
| Protein S100-A6; Protein S100 | S100A6 | −2.610746384 | 0.011829 |
| Isocitrate dehydrogenase [NADP] | IDH1 | −2.627195994 | 0.017449 |
| cytoplasmic | | | |
| UTP--glucose-1-phosphate | UGP2 | −2.646575292 | 0.002091 |
| uridylyltransferase | | | |
| Stromal cell-derived factor 1; SDF-1-beta(3- | CXCL12 | −2.656158447 | 0.010785 |
| 72); SDF-1-alpha(3-67) | | | |
| IST1 homolog | IST1 | −2.673505147 | 0.011028 |
| Collagen alpha-2(I) chain | COL1A2 | −2.676224391 | 0.031419 |
| EH domain-containing protein 4 | EHD4 | −2.737970352 | 0.011052 |
| Annexin A6; Annexin | ANXA6 | −2.758201599 | 0.00036 |
| N(G),N(G)-dimethylarginine | DDAH2 | −2.759220759 | 0.000313 |
| dimethylaminohydrolase 2 | | | |
| Dihydropyrimidinase-related protein 2 | DPYSL2 | −2.791438421 | 0.000903 |
| Galectin-1 | LGALS1 | −2.815954208 | 0.003379 |
| Ras-related protein Rab-2A; Ras-related | RAB2A; | −2.867925644 | 0.003032 |
| protein Rab-2B | RAB2B | | |
| Matrix metalloproteinase-14 | MMP14 | −2.910762787 | 0.000205 |
| Myeloid-associated differentiation marker | MYADM | −2.93177096 | 0.0057 |
| Ras-related protein Ral-A | RALA | −2.96500206 | 0.000361 |
| Tropomyosin alpha-4 chain | TPM4 | −3.021409353 | 0.00097 |
| Transferrin receptor protein 1; Transferrin | TFRC | −3.028935115 | 0.010454 |
| receptor protein 1, serum form | | | |
| Histone H4 | HIST1H4A | −3.030223211 | 0.00877 |
| Histone H3.1 | HIST1H3A | −3.073619843 | 0.012832 |
| Fibulin-1 | FBLN1 | −3.091328939 | 0.000389 |
| Dihydropyrimidinase-related protein 3 | DPYSL3 | −3.097085317 | 0.000547 |
| Synaptic vesicle membrane protein VAT-1 | VAT1 | −3.165213267 | 0.004332 |
| homolog | | | |
| Vimentin | VIM | −3.167142232 | 0.003979 |
| Integrin beta-5 | ITGB5 | −3.24244372 | 0.005679 |
| Versican core protein | VCAN | −3.298335393 | 7.23E−05 |
| Band 4.1-like protein 2 | EPB41L2 | −3.298443794 | 0.00035 |
| Ubiquitin carboxyl-terminal hydrolase | UCHL1 | −3.346582413 | 0.007431 |
| isozyme L1; Ubiquitin carboxyl-terminal | | | |
| hydrolase | | | |
| 72 kDa type IV collagenase; PEX | MMP2 | −3.526647568 | 0.001642 |
| Collagen alpha-1 (I) chain | COL1A1 | −3.535470327 | 0.013021 |
| D-3-phosphoglycerate dehydrogenase | PHGDH | −3.591803233 | 0.000123 |
| Transketolase | TKT | −3.647360484 | 0.000639 |
| Ras-related protein Rap-2b | RAP2B | −3.728734334 | 0.001711 |
| Fatty acid synthase; [Acyl-carrier-protein] S- | FASN | −3.753777822 | 0.007852 |
| acetyltransferase; [Acyl-carrier-protein] S- | | | |
| malonyltransferase; 3-oxoacyl-[acyl-carrier- | | | |
| protein] synthase; 3-oxoacyl-[acyl-carrier- | | | |
| protein] reductase; 3-hydroxyacyl-[acyl- | | | |
| carrier-protein] dehydratase; Enoyl-[acyl- | | | |
| carrier-protein] reductase; Oleoyl-[acyl- | | | |
| carrier-protein] hydrolase | | | |
| Nucleoside diphosphate kinase; Nucleoside | NME1-NME2; | −3.81598409 | 0.006383 |
| diphosphate kinase B | NME2 | | |
| Myristoylated alanine-rich C-kinase substrate | MARCKS | −3.963729858 | 0.025408 |
| Biglycan | BGN | −3.976584752 | 0.019814 |
| Histone H1.5 | HIST1H1B | −4.113817215 | 0.001408 |
| Thrombospondin-2 | THBS2 | −4.159285863 | 0.002696 |
| Annexin A4; Annexin | ANXA4 | −4.417996724 | 0.00184 |
| Niban-like protein 1 | FAM129B | −4.425540288 | 5.34E−05 |
| Afamin | AFM | −4.510639826 | 0.001023 |
| Target of Nesh-SH3 | ABI3BP | −4.569406827 | 0.004832 |
| Pro-low-density lipoprotein receptor-related | LRP1 | −4.599747976 | 0.000602 |
| protein 1; Low-density lipoprotein receptor- | | | |
| related protein 1 85 kDa subunit; Low-density | | | |
| lipoprotein receptor-related protein 1 515 | | | |
| kDa subunit; Low-density lipoprotein | | | |
| receptor-related protein 1 intracellular | | | |
| domain | | | |
| Neuroblast differentiation-associated protein | AHNAK | −4.833908081 | 0.018608 |
| AHNAK | | | |
| Serpin H1 | SERPINH1 | −4.999172211 | 0.000417 |
| Guanine nucleotide-binding protein G(k) | GNAI3 | −5.091183027 | 0.0021 |
| subunit alpha | | | |
| Myoferlin | MYOF | −5.710025152 | 0.026799 |

TABLE 9 shows the top 10 BP GO enrichment terms of HDC EV protein cargo;

HDC

| | | | Fold |
|---|---|---|---|
| GO term | Count | P-Value | Enrichment |
| GO:0007155~cell adhesion | 108 | 1.45E−36 | 4.080501516 |
| GO:0002181~cytoplasmic | 40 | 4.75E−28 | 9.35324833 |
| translation | | | |
| GO:0050821~protein stabilization | 47 | 8.64E−18 | 4.495936413 |
| GO:0006412~translation | 43 | 1.34E−14 | 4.057967605 |
| GO:0070527~platelet aggregation | 20 | 4.47E−14 | 9.35324833 |
| GO:0016192~vesicle-mediated | 41 | 1.33E−12 | 3.70316377 |
| transport | | | |
| GO:0030335~positive regulation | 43 | 1.73E−12 | 3.534870219 |
| of cell migration | | | |
| GO:0036258~multivesicular body | 16 | 1.84E−12 | 10.86183677 |
| assembly | | | |
| GO:0007160~cell-matrix adhesion | 27 | 3.62E−12 | 5.3103723 |
| GO:0072659~protein localization | 33 | 4.27E−12 | 4.313532227 |
| to plasma membrane | | | |

TABLE 10 shows the top 10 BP GO enrichment terms of BM MSC protein cargo;

BM-MSC

| | | | Fold |
|---|---|---|---|
| GO term | Count | P-Value | Enrichment |
| GO:0002181~cytoplasmic | 54 | 5.96E−47 | 12.68232711 |
| translation | | | |
| GO:0006412~translation | 58 | 1.77E−26 | 5.497570796 |
| GO:0007155~cell adhesion | 82 | 7.10E−20 | 3.111761889 |
| GO:0050821~protein stabilization | 48 | 1.28E−18 | 4.611755314 |
| GO:0016192~vesicle-mediated | 48 | 1.44E−17 | 4.354447077 |
| transport | | | |
| GO:0006886~intracellular protein | 51 | 2.06E−13 | 3.286578673 |
| transport | | | |
| GO:0006457~protein folding | 35 | 3.25E−13 | 4.456641054 |
| GO:0070527~platelet aggregation | 19 | 6.01E−13 | 8.924600561 |
| GO:0015031~protein transport | 57 | 1.29E−11 | 2.744467143 |
| GO:1900026~positive regulation | 17 | 6.51E−11 | 8.166650035 |
| of substrate adhesion-dependent | | | |
| cell spreading | | | |

TABLE 11 shows the top 10 BP GO enrichment terms of UC MSC EV protein cargo;

UC-MSC

| | | | Fold |
|---|---|---|---|
| GO term | Count | P-Value | Enrichment |
| GO:0007596~blood coagulation | 36 | 8.01E-27 | 10.61652 |
| GO:0006958~complement activation, | 39 | 3.28E-24 | 8.16216 |
| classical pathway | | | |
| GO:0010951~negative regulation of | 41 | 2.22E-23 | 7.287745 |
| endopeptidase activity | | | |
| GO:0070527~platelet aggregation | 25 | 1.31E-22 | 14.41749 |
| GO:0007155~cell adhesion | 71 | 1.25E-18 | 3.307999 |
| GO:0006956~complement activation | 17 | 2.86E-17 | 17.64701 |
| GO:0007160~cell-matrix adhesion | 29 | 3.35E-16 | 7.033579 |
| GO:0030036~actin cytoskeleton | 37 | 4.34E-16 | 5.275851 |
| organization | | | |
| GO:0007229~integrin-mediated | 28 | 7.12E-15 | 6.605832 |
| signaling pathway | | | |
| GO:0042730~fibrinolysis | 14 | 8.00E-15 | 19.12214 |

TABLE 12 shows a comparison of GO enrichment terms for BM-MSC, HDC, and UC-MSC EV protein cargo;

| | |
|---|---|
| Category | # of enriched Go terms |

HDC vs. BM-MSC EV

| | |
|---|---|
| Biological process (BP) | 6 |
| Cellular component (CC) | 19 |
| Molecular function (MF) | 7 |

BM-MSC vs. UC-MSC EV

| | |
|---|---|
| Biological process (BP) | 360 |
| Cellular component (CC) | 171 |
| Molecular function (MF) | 105 |

HDC vs. UC-MSC EV

| | |
|---|---|
| Biological process (BP) | 312 |
| Cellular component (CC) | 147 |
| Molecular function (MF) | 87 |

As used herein, the term “about” refers to an approximately +/−10% variation from a given value. It is to be understood that such a variation is always included in any given value provided herein, whether or not it is specifically referred to.

As used herein, the term “substantially” refers to an approximately +/−5% variation from a given value. If a value is not used, then substantially means almost completely, but perhaps with some variation, contamination and/or additional component. In some embodiments, “substantially” may include completely.

All references cited herein are herein incorporated by reference in their entirety.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

One or more illustrative embodiments have been described by way of example. It will be understood to persons skilled in the art that a number of variations and modifications may be made without departing from the scope of the invention as defined in the claims.

### REFERENCES

Adam, O., B. Lohfelm, et al. (2012). “Role of miR-21 in the pathogenesis of atrial fibrosis.” Basic Res Cardiol 107(5): 278.
Aguilar, M., X. Y. Qi, et al. (2014). “Fibroblast electrical remodeling in heart failure and potential effects on atrial fibrillation.” Biophys J 107(10): 2444-2455.
Ashihara, T., R. Haraguchi, et al. (2012). “The role of fibroblasts in complex fractionated electrograms during persistent/permanent atrial fibrillation: implications for electrogram-based catheter ablation.” Circ Res 110(2): 275-284.
Baghy, K., R. V. Iozzo, et al. (2012). “Decorin-TGFbeta axis in hepatic fibrosis and cirrhosis.” J Histochem Cytochem 60(4): 262-268.
Balafkan, N., S. Mostafavi, et al. (2020). “A method for differentiating human induced pluripotent stem cells toward functional cardiomyocytes in 96-well microplates.” Sci Rep 10(1): 18498.
Barile, L., V. Lionetti, et al. (2014). “Extracellular vesicles from human cardiac progenitor cells inhibit cardiomyocyte apoptosis and improve cardiac function after myocardial infarction.” Cardiovasc Res 103(4): 530-541.
Benjamin, E. J., D. Levy, et al. (1994). “Independent risk factors for atrial fibrillation in a population-based cohort. The Framingham Heart Study.” JAMA 271(11): 840-844.
Boehncke, W. H. and N. C. Brembilla (2018). “Immunogenicity of biologic therapies: causes and consequences.” Expert Rev Clin Immunol 14(6): 513-523.
Bretones, G., M. D. Delgado, et al. (2015). “Myc and cell cycle control.” Biochim Biophys Acta 1849(5): 506-516.
Burgmaier, M., K. Schutters, et al. (2014). “AnxA5 reduces plaque inflammation of advanced atherosclerotic lesions in apoE(−/−) mice.” J Cell Mol Med 18(10): 2117-2124.
Canesi, F., V. Mateo, et al. (2019). “A thioredoxin-mimetic peptide exerts potent anti-inflammatory, antioxidant, and atheroprotective effects in ApoE2.Ki mice fed high fat diet.” Cardiovasc Res 115(2): 292-301.
Cao, W., P. Shi, et al. (2017). “miR-21 enhances cardiac fibrotic remodeling and fibroblast proliferation via CADM1/STAT3 pathway.” BMC Cardiovasc Disord 17(1): 88.
Cardin, S., E. Guasch, et al. (2012). “Role for MicroRNA-21 in atrial profibrillatory fibrotic remodeling associated with experimental postinfarction heart failure.” Circ Arrhythm Electrophysiol 5(5): 1027-1035.
Chiang, C. Y. and C. Chen (2019). “Toward characterizing extracellular vesicles at a single-particle level.” J Biomed Sci 26(1): 9.
Ciferri, M. C., R. Quarto, et al. (2021). “Extracellular Vesicles as Biomarkers and Therapeutic Tools: From Pre-Clinical to Clinical Applications.” Biology (Basel) 10(5).
Correa, S. G., C. E. Sotomayor, et al. (2003). “Opposite effects of galectin-1 on alternative metabolic pathways of L-arginine in resident, inflammatory, and activated macrophages.” Glycobiology 13(2): 119-128.
Cox, J. and M. Mann (2008). “MaxQuant enables high peptide identification rates, individualized p.p.b.-range mass accuracies and proteome-wide protein quantification.” Nat Biotechnol 26(12): 1367-1372.
Davis, D. R., E. Kizana, et al. (2010). “Isolation and expansion of functionally-competent cardiac progenitor cells directly from heart biopsies.” J Mol Cell Cardiol 49(2): 312-321. de Jong, R. C. M., N. J. Pluijmert, et al. (2018). “Annexin A5 reduces infarct size and improves cardiac function after myocardial ischemia-reperfusion injury by suppression of the cardiac inflammatory response.” Sci Rep 8(1): 6753.
El Hadri, K., D. F. Mahmood, et al. (2012). “Thioredoxin-1 promotes anti-inflammatory macrophages of the M2 phenotype and antagonizes atherosclerosis.” Arterioscler Thromb Vasc Biol 32(6): 1445-1452.
El Harane, N., Kervadec, A., et al. (2018). “Acellular therapeutic approach for heart failure: in vitro production of extracellular vesicles from human cardiovascular progenitors”. Eur Heart J 39(20):1835-1847 Escudier, B., T. Dorval, et al. (2005). “Vaccination of metastatic melanoma patients with autologous dendritic cell (DC) derived-exosomes: results of the first phase I clinical trial.” J Transl Med 3(1): 10.
Ewing, M. M., M. R. de Vries, et al. (2011). “Annexin A5 therapy attenuates vascular inflammation and remodeling and improves endothelial function in mice.” Arterioscler Thromb Vasc Biol 31(1): 95-101.
Friberg, L. (2018). “Ventricular arrhythmia and death among atrial fibrillation patients using anti-arrhythmic drugs.” Am Heart J 205: 118-127.
Furberg, C. D., B. M. Psaty, et al. (1994). “Prevalence of atrial fibrillation in elderly subjects (the Cardiovascular Health Study).” Am J Cardiol 74(3): 236-241.
Girmatsion, Z., P. Biliczki, et al. (2009). “Changes in microRNA-1 expression and IK1 up-regulation in human atrial fibrillation.” Heart Rhythm 6(12): 1802-1809.
Gray, A., Wright, A., et al. (2015). “Quantification of histochemical stains using whole slide imaging: development of a method and demonstration of its usefulness in laboratory quality control.” J Clin Pathol 68(3):192-9.
Go, A. S., E. M. Hylek, et al. (2001). “Prevalence of diagnosed atrial fibrillation in adults: national implications for rhythm management and stroke prevention: the AnTicoagulation and Risk Factors in Atrial Fibrillation (ATRIA) Study.” JAMA 285(18): 2370-2375.
Hall, J., S. Prabhakar, et al. (2016). “Delivery of Therapeutic Proteins via Extracellular Vesicles: Review and Potential Treatments for Parkinson's Disease, Glioma, and Schwannoma.” Cell Mol Neurobiol 36(3): 417-427.
He, X., X. Gao, et al. (2011). “Atrial fibrillation induces myocardial fibrosis through angiotensin II type 1 receptor-specific Arkadia-mediated downregulation of Smad7.” Circ Res 108(2): 164-175.
Huang, Z., X. J. Chen, et al. (2016). “Signal Transducer and Activator of Transcription 3/MicroRNA-21 Feedback Loop Contributes to Atrial Fibrillation by Promoting Atrial Fibrosis in a Rat Sterile Pericarditis Model.” Circ Arrhythm Electrophysiol 9(7).
Hur, J. Y. and K. Y. Lee (2021). “Characteristics and Clinical Application of Extracellular Vesicle-Derived DNA.” Cancers (Basel) 13(15).
Jamall, I. S., V. N. Finelli, et al. (1981). “A simple method to determine nanogram levels of 4-hydroxyproline in biological tissues.” Anal Biochem 112(1): 70-75.
Jost, N., T. Christ, et al. (2021). “New Strategies for the Treatment of Atrial Fibrillation.” Pharmaceuticals (Basel) 14(9).
Jung, J. Y., Y. H. Kwak, et al. (2017). “Protective effect of hemopexin on systemic inflammation and acute lung injury in an endotoxemia model.” J Surg Res 212: 15-21.
Kanda, P., E. I. Alarcon, et al. (2018). “Deterministic Encapsulation of Human Cardiac Stem Cells in Variable Composition Nanoporous Gel Cocoons To Enhance Therapeutic Repair of Injured Myocardium.” ACS Nano 12(5): 4338-4350.
Kanda, P., E. I. Alarcon, et al. (2018). “Deterministic Encapsulation of Human Cardiac Stem Cells in Variable Composition Nanoporous Gel Cocoons To Enhance Therapeutic Repair of Injured Myocardium.” ACS Nano.
Kapoor, N., W. Liang, et al. (2013). “Direct conversion of quiescent cardiomyocytes to pacemaker cells by expression of Tbx18.” Nat Biotechnol 31(1): 54-62.
Kendall, R. T. and C. A. Feghali-Bostwick (2014). “Fibroblasts in fibrosis: novel roles and mediators.” Front Pharmacol 5: 123.
Konoshenko, M. Y., E. A. Lekchnov, et al. (2018). “Isolation of Extracellular Vesicles: General Methodologies and Latest Trends.” Biomed Res Int 2018: 8545347.
Kooijmans, S. A. A., R. M. Schiffelers, et al. (2016). “Modulation of tissue tropism and biological activity of exosomes and other extracellular vesicles: New nanotools for cancer treatment.” Pharmacol Res 111: 487-500.
Latham, N., B. Ye, et al. (2013). “Human blood and cardiac stem cells synergize to enhance cardiac repair when cotransplanted into ischemic myocardium.” Circulation 128(11 Suppl 1): S105-112.
Ledan S (2020). “Antiarrhythmic Treatment in Atrial Fibrillation.” US Pharm. 45(2):24-27.
Liang, X., T. Lin, et al. (2009). “Hemopexin down-regulates LPS-induced proinflammatory cytokines from macrophages.” J Leukoc Biol 86(2): 229-235.
Liang, Y., Yuan, W., et al. (2015). “Macrophage migration inhibitory factor promotes expression of GLUT4 glucose transporter through MEF2 and ZacI in cardiomyocytes.” Metabolism 64(12):1682-93.
Lim, L. H., E. Solito, et al. (1998). “Promoting detachment of neutrophils adherent to murine postcapillary venules to control inflammation: effect of lipocortin 1.” Proc Natl Acad Sci USA 95(24): 14535-14539.
Liu, Y., S. Chen, et al. (2017). “Transcriptional landscape of the human cell cycle.” Proc Natl Acad Sci USA 114(13): 3473-3478.
Lotvall, J., A. F. Hill, et al. (2014). “Minimal experimental requirements for definition of extracellular vesicles and their functions: a position statement from the International Society for Extracellular Vesicles.” J Extracell Vesicles 3: 26913.
Ludhwani, D., A. Goyal, et al. (2021). “Ventricular Fibrillation.”
Luo, X., Z. Pan, et al. (2013). “MicroRNA-26 governs profibrillatory inward-rectifier potassium current changes in atrial fibrillation.” J Clin Invest 123(5): 1939-1951.
Lycke, M., L. O'Neill, et al. (2021). “How Close Are We toward an Optimal Balance in Safety and Efficacy in Catheter Ablation of Atrial Fibrillation? Lessons from the CLOSE Protocol.” J Clin Med 10(18).
McRae, C., A. Kapoor, et al. (2019). “Systematic review of biological therapies for atrial fibrillation.” Heart Rhythm 16(9): 1399-1407.
Merico, D., R. Isserlin, et al. (2010). “Enrichment map: a network-based method for gene-set enrichment visualization and interpretation.” PLoS One 5(11): e13984.
Mount, S., P. Kanda, et al. (2019). “Physiologic expansion of human heart-derived cells enhances therapeutic repair of injured myocardium.” Stem Cell Res Ther 10(1): 316.
Murphy, D. E., O. G. de Jong, et al. (2019). “Extracellular vesicle-based therapeutics: natural versus engineered targeting and trafficking.” Exp Mol Med 51(3): 1-12.
Narikawa, M., M. Umemura, et al. (2018). “Acute Hyperthermia Inhibits TGF-betal-induced Cardiac Fibroblast Activation via Suppression of Akt Signaling.” Sci Rep 8(1): 6277.
Nesheiwat, Z., A. Goyal, et al. (2021). “Atrial Fibrillation.”
Onozuka, I., S. Kakinuma, et al. (2011). “Cholestatic liver fibrosis and toxin-induced fibrosis are exacerbated in matrix metalloproteinase-2 deficient mice.” Biochem Biophys Res Commun 406(1): 134-140.
Parichatikanond, W., T. Luangmonkong, et al. (2020). “Therapeutic Targets for the Treatment of Cardiac Fibrosis and Cancer: Focusing on TGF-beta Signaling.” Front Cardiovasc Med 7: 34.
Park, K. S., E. Bandeira, et al. (2019). “Enhancement of therapeutic potential of mesenchymal stem cell-derived extracellular vesicles.” Stem Cell Res Ther 10(1): 288.
Pfeifer, P., N. Werner, et al. (2015). “Role and Function of MicroRNAs in Extracellular Vesicles in Cardiovascular Biology.” Biomed Res Int 2015: 161393.
Quattrocelli, M., S. Crippa, et al. (2013). “Long-term miR-669a therapy alleviates chronic dilated cardiomyopathy in dystrophic mice.” J Am Heart Assoc 2(4): e000284.
Radbill, B. D., R. Gupta, et al. (2011). “Loss of matrix metalloproteinase-2 amplifies murine toxin-induced liver fibrosis by upregulating collagen I expression.” Dig Dis Sci 56(2): 406-416.
Risha, Y., Z. Minic, et al. (2020). “The proteomic analysis of breast cell line exosomes reveals disease patterns and potential biomarkers.” Sci Rep 10(1): 13572.
Rizvi, F., A. DeFranco, et al. (2016). “Chamber-specific differences in human cardiac fibroblast proliferation and responsiveness toward simvastatin.” Am J Physiol Cell Physiol 311(2): C330-339.
Rubin, S. M., J. Sage, et al. (2020). “Integrating Old and New Paradigms of G1/S Control.” Mol Cell 80(2): 183-192.
Seo, C., C. Michie, et al. (2020). “Systematic review of pre-clinical therapies for post-operative atrial fibrillation.” PLoS One 15(11): e0241643.
Shan, H., Y. Zhang, et al. (2009). “Downregulation of miR-133 and miR-590 contributes to nicotine-induced atrial remodelling in canines.” Cardiovasc Res 83(3): 465-472.
Sidiropoulos, K., G. Viteri, et al. (2017). “Reactome enhanced pathway visualization.” Bioinformatics 33(21): 3461-3467.
Small, E. M., R. J. Frost, et al. (2010). “MicroRNAs add a new dimension to cardiovascular disease.” Circulation 121(8): 1022-1032.
Suetsugu, A., K. Honma, et al. (2013). “Imaging exosome transfer from breast cancer cells to stroma at metastatic sites in orthotopic nude-mouse models.” Adv Drug Deliv Rev 65(3): 383-390.
Thery, C., K. W. Witwer, et al. (2018). “Minimal information for studies of extracellular vesicles 2018 (MISEV2018): a position statement of the International Society for Extracellular Vesicles and update of the MISEV2014 guidelines.” J Extracell Vesicles 7(1): 1535750.
Tsoporis, J. N., A. Fazio, et al. (2018). “Increased right atrial appendage apoptosis is associated with differential regulation of candidate MicroRNAs 1 and 133A in patients who developed atrial fibrillation after cardiac surgery.” J Mol Cell Cardiol 121: 25-32.
van Rooij, E., L. B. Sutherland, et al. (2008). “Dysregulation of microRNAs after myocardial infarction reveals a role of miR-29 in cardiac fibrosis.” Proc Natl Acad Sci USA 105(35): 13027-13032.
Villanueva, M., C. Michie, et al. (2019). “Glyoxalase 1 Prevents Chronic Hyperglycemia Induced Heart-Explant Derived Cell Dysfunction.” Theranostics 9(19): 5720-5730.
Vital, S. A., F. Becker, et al. (2016). “Formyl-Peptide Receptor 2/3/Lipoxin A4 Receptor Regulates Neutrophil-Platelet Aggregation and Attenuates Cerebral Inflammation: Impact for Therapy in Cardiovascular Disease.” Circulation 133(22): 2169-2179.
Welsh, J. A. and J. C. Jones (2020). “Small Particle Fluorescence and Light Scatter Calibration Using FCMPASS Software.” Curr Protoc Cytom 94(1): e79.
Welsh, J. A., J. C. Jones, et al. (2020). “Fluorescence and Light Scatter Calibration Allow Comparisons of Small Particle Data in Standard Units across Different Flow Cytometry Platforms and Detector Settings.” Cytometry A 97(6): 592-601.
Wynn, T. A. (2008). “Cellular and molecular mechanisms of fibrosis.” J Pathol 214(2): 199-210.
Yang, H., X. Qin, et al. (2019). “An in Vivo miRNA Delivery System for Restoring Infarcted Myocardium.” ACS Nano 13(9): 9880-9894.
Yue, L., J. Xie, et al. (2011). “Molecular determinants of cardiac fibroblast electrical function and therapeutic implications for atrial fibrillation.” Cardiovasc Res 89(4): 744-753.
Zhi, Y., C. Xu, et al. (2019). “Effective Delivery of Hypertrophic miRNA Inhibitor by Cholesterol-Containing Nanocarriers for Preventing Pressure Overload Induced Cardiac Hypertrophy.” Adv Sci (Weinh) 6(11): 1900023.
Zhou, X. and S. C. Dudley, Jr. (2020). “Evidence for Inflammation as a Driver of Atrial Fibrillation.” Front Cardiovasc Med 7: 62.
Zimta, A. A., O. E. Sigurjonsson, et al. (2020). “The Malignant Role of Exosomes as Nanocarriers of Rare RNA Species.” Int J Mol Sci 21(16).
Lu M, Shi B, Wang J, Cao Q and Cui Q. TAM: a method for enrichment and depletion analysis of a microRNA category in a list of microRNAs. BMC Bioinformatics. 2010; 11:419.
Li J, Han X, Wan Y, Zhang S, Zhao Y, Fan R, Cui Q and Zhou Y. TAM 2.0: tool for MicroRNA set analysis. Nucleic Acids Res. 2018; 46:W180-wl85.
Sherman B T, Hao M, Qiu J, Jiao X, Baseler M W, Lane H C, Imamichi T and Chang W. DAVID: a web server for functional enrichment analysis and functional annotation of gene lists (2021 update).
Nucleic Acids Res. 2022; 50:W216-21.
Huang da W, Sherman B T and Lempicki R A. Systematic and integrative analysis of large gene lists using DAVID bioinformatics resources. Nat Protoc. 2009; 4:44-57.
Sticht C, De La Torre C, Parveen A and Gretz N. miRWalk: An online resource for prediction of microRNA binding sites. PLoS One. 2018; 13:e0206239.
Szklarczyk D, Gable A L, Lyon D, Junge A, Wyder S, Huerta-Cepas J, Simonovic M, Doncheva N T, Morris J H, Bork P, et al. STRING vii: protein-protein association networks with increased coverage, supporting functional discovery in genome-wide experimental datasets. Nucleic Acids Res. 2019; 47:D607-d613.
Kanda P, Benavente-Babace A, Parent S, Connor M, Soucy N, Steeves A, Lu A, Cober N D, Courtman D, Variola F, et al. Deterministic paracrine repair of injured myocardium using microfluidic-based cocooning of heart explant-derived cells. Biomaterials. 2020; 247:120010.
McIntyre L A, Stewart D J, Mei S H J, Courtman D, Watpool I, Granton J, Marshall J, Dos Santos C, Walley K R, Winston B W, et al. Cellular Immunotherapy for Septic Shock. A Phase I Clinical Trial. Am J Respir Crit Care Med. 2018; 197:337-347.
Schlosser K, Wang J P, Dos Santos C, Walley K R, Marshall J, Fergusson D A, Winston B W, Granton J, Watpool I, Stewart D J, et al. Effects of Mesenchymal Stem Cell Treatment on Systemic Cytokine Levels in a Phase 1 Dose Escalation Safety Trial of Septic Shock Patients. Crit Care Med. 2019; 47:918-925.
Vaka R, Khan S, Ye B, Risha Y, Parent S, Courtman D, Stewart D J and Davis D R. Direct comparison of different therapeutic cell types susceptibility to inflammatory cytokines associated with COVID-19 acute lung injury. Stem Cell Res Ther. 2022; 13:20.
English S, Fergusson D, Lalu M, Thebaud B, Watpool I, Champagne J, Sobh M, Courtman D W, Khan S, Jamieson M, et al. Results of the cellular immuno-therapy for covid-19 related acute respiratory distress syndrome (circa-phase i trial: Cytotherapy. 2021 May; 23(5):S22. doi: 10.1016/S1465324921002796. Epub 2021 May 11.
Bandettini W P, Kellman P, Mancini C, Booker O J, Vasu S, Leung S W, Wilson J R, Shanbhag S M, Chen M Y and Arai A E. MultiContrast Delayed Enhancement (MCODE) improves detection of subendocardial myocardial infarction by late gadolinium enhancement cardiovascular magnetic resonance: a clinical validation study. J Cardiovasc Magn Reson. 2012; 14:83.
Tyanova S, Temu T, Sinitcyn P, Carlson A, Hein M Y, Geiger T, Mann M, Cox, J. The Perseus computational platform for comprehensive analysis of (prote)omics data. Nat Methods. 2016 September; 13(9):731-40. doi: 10.1038/nmeth.3901. Epub 2016 Jun. 27.
Tilokee E L, Latham N, Jackson R, Mayfield A E, Ye B, Mount S, Lam B K, Suuronen E J, Ruel M, Stewart D J, Davis D R, Paracrine Engineering of Human Explant-Derived Cardiac Stem Cells to Over-Express Stromal-Cell Derived Factor 1alpha Enhances Myocardial Repair. Stem Cells. 2016 July; 34(7):1826-35. doi: 10.1002/stem.2373. Epub 2016 Apr. 21.
Jackson R, Tilokee E L, Latham N, Mount S, Rafatian G, Strydhorst J, Ye B, Boodhwani M, Chan V, Ruel M, Ruddy T D, Suuronen E J, Stewart D J, Davis D R, Paracrine Engineering of Human Cardiac Stem Cells With Insulin-Like Growth Factor 1 Enhances Myocardial Repair. J Am Heart Assoc. 2015; 4. J Am Heart Assoc. 2015 Sep. 11; 4(9):e002104. doi: 10.1161/JAHA.115.002104.

1.-163. (canceled)

164. A composition comprising extracellular vesicles isolated from human heart cells, the vesicles comprising a polydisperse population of particles with a diameter ranging from about 10 to about 500 nm and comprising miR-199, miR-22-3p and miR-374a-5p.

165. The composition of claim 164 wherein the vesicles comprise acetylcholinesterase activity.

166. The composition of claim 164 wherein the vesicles comprise 1-1000 unique miRNA transcripts.

167. The composition of claim 164 in a unit dosage of extracellular vesicles comprising 10²-10²⁰ particles per millilitre.

168. The composition of claim 164 wherein said extracellular vesicles decrease inflammasome activation.

169. The composition of claim 164, wherein the human heart cells are derived from myocardial tissue.

170. The composition of claim 169, wherein the human heart cells are derived from an atrial appendage.

171. The composition of claim 164 wherein the human heart cells are grown in-vitro.

172. The composition of claim 164 comprising one or more transmembrane markers defined by CD9, CD61, CD63, CD81, FLOT1, ICAM1, EpCam, or any combination thereof.

173. The composition of claim 164 comprising one or more cytosolic markers defined by ALIX, ANXA5 and TSG101, or any combination thereof.

174. The composition of claim 164 substantially lacking or devoid of GM130.

175. The composition of claim 164, wherein the vesicles prevent or reduce atrial inflammation.

176. The composition of claim 164 for treating atrial arrhythmia, or preventing or reducing atrial inflammation.
